(19) United States
(12) Patent Application Publication
(10) Pub. No.: US 2025/0205467 A1
(43) Pub. Date: Jun. 26, 2025

(54) SYSTEMS AND METHODS FOR THE TREATMENT OF CANCER USING ULTRASOUND

(71) Applicants: Duke University, Durham, NC (US); North Carolina State University, Raleigh, NC (US)

(72) Inventors: Herbert Kim Lyerly, Chapel Hill, NC (US); Takuya Osada, Durham, NC (US); Zachary C. Hartman, Durham (NC); Pei Zhong, Durham, NC (US); Kamran Mahmood, Durham, NC (US); Xiaoning Jiang, Cary, NC (US); Howuk Kim, San Jose, CA (US)

(21) Appl. No.: 18/851,640

(22) PCT Filed: Mar. 28, 2023
(86) PCT No.: PCT/US2023/016636

§ 371 Date: Sep. 26, 2024

Related U.S. Application Data
(60) Provisional application No. 63/324,479, filed on Mar. 28, 2022.

(51) Int. Cl. A61M 37/00; A61K 38/20; A61K 41/00; A61M 25/00; A61N 7/00; A61N 7/02

(52) U.S. Cl. CPC A61M 37/0092; A61K 38/208; A61K 41/0047; A61M 25/0026; A61N 7/02; A61M 2037/0007; A61M 2202/07; A61N 2007/003; A61N 2007/0039

(57) ABSTRACT

A novel localized, mechanical HIFU (LM-HIFU) transcatheter device that can ablate cancer cells and disrupt the stromal barrier in tumors, enhancing the efficacy of therapeutics and increasing immune cell infiltration. A miniaturized dual-lumen catheter device is configured to deliver M-HIFU to a tumor that does not have the anatomic limitations of conventional HIFU, and enables access to a primary or metastatic tumor regardless of the location.

### CROSS-REFERENCE TO RELATED APPLICATIONS

This application is a non-provisional of and claims the benefit of U.S. Provisional Patent Application No. 63/324,479, filed on Mar. 28, 2022, the contents of which are incorporated herein by reference.

### BACKGROUND

Solid tumors constitute the vast majority of cancers, with more than 1.5 million new cases diagnosed per year in the U.S. Common treatments include surgery, ablation, radiotherapy, chemotherapy, and more recent advances in immunotherapy and other targeted therapies, which can be delivered either alone or as combination therapies.

Immunotherapy has emerged as a treatment for cancer, primarily consisting of immune checkpoint blockade or cellular therapies. Despite the promise of systemic immunotherapy, it is effective in less than 15% of patients and has significant side effects that impact patients' quality of life. Therefore, the majority of patients with metastatic disease still do not benefit from immunotherapies and combination therapies.

Cancer immunotherapy using immune checkpoint blockade (“ICB”) has revolutionized cancer therapy and is a major focus in cancer research. Yet, this method of treatment still has several drawbacks, including relatively low response rates. Therefore, there is a need to convert immunologically “cold tumors” to inflamed “hot tumors” in immune therapy. Because the majority of patients do not respond to ICB alone, combinations of immunotherapy delivered systemically have been investigated, and these not only increase tumor response rates, but also increase toxicity and treatment related death rate.

While numerous attempts to dissociate systemic toxicity with the increase in antitumor immune responses are being explored, this has not been achieved except using one modality-intralesional therapy. Intralesional therapy, or the local delivery of agents into the tumor, includes oncolytic viruses and other immunologic agents that have demonstrated the ability to stimulate antitumor responses with minimal if any systemic toxicity. Unfortunately, the intratumoral delivery of these agents is typically by needle injection and this has several limitations: 1) needle injection has several drawbacks, such as failed injections (e.g., missing the tumor, leakage of drug to surrounding tissues, little drug delivered to tumor, etc.) and 2) the response rates are low, often less than 33%.

Additionally, a persistent barrier to treatment includes the complex and dense nature of the tumor stroma and the high interstitial tissue pressure within primary and metastatic tumors, which inhibits the delivery of therapeutics, and limits activated immune cells to penetrate.

Local ablation techniques, such as high intensity focused ultrasound (HIFU), are being employed to thermally destroy cancer cells, and increasingly being explored to augment cancer immunotherapy. HIFU is currently delivered by sound waves that originate outside the body, but the sound waves cannot traverse air-fluid interfaces. Thus, current HIFU strategies have anatomic limitations, preventing them from treating common organs of advanced cancer metastasis, such as the lung, airways or tumors within the gastrointestinal (GI) tract.

Thus, there is an ongoing need for improved approaches to treating cancer.

### SUMMARY

As disclosed herein, a micro-transducer-based catheter uses a frequency and energy level which are configured to puncture tumor cell membranes, disrupt the tumor stroma, improve the uptake of therapeutics, and promote immune cell infiltration. To address the anatomic limitations of conventional HIFU, and the unmet need for a more effective means of tumor ablation and delivery of anti-cancer therapeutics directly into tumors, the micro-transducer-based catheter is configured to deliver high-intensity focused ultrasound (HIFU) to disrupt the tumor microenvironment, and simultaneously deliver a therapeutic into the tumor, taking advantage of the synergism between tumor ablation immunotherapy. HIFU-induced mechanical disruption of a targeted tissue by acoustic cavitation and disruption of cell membranes is known as histotripsy and is achieved through the high-pressure bursting of microbubbles that are induced by the ultrasound treatment.

In one embodiment, the disclosure provides a novel localized, mechanical HIFU (LM-HIFU) transcatheter device that can ablate cancer cells and disrupt the stromal barrier in tumors, enhancing the efficacy of therapeutics and increasing immune cell infiltration. Using a miniaturized device to deliver M-HIFU as described herein does not have the anatomic limitations of conventional HIFU and enables access to a primary or metastatic tumor regardless of the location.

One aspect of the present disclosure provides a system for the treatment of a target (e.g., tumor tissue, peritumoral tissue, non-malignant tissue, hematologic cells, or immune cells) the system comprising (a) an ultrasound energy source; (b) a device coupled to the ultrasound energy source and configured and arranged to: (i) direct the energy to a desired location; (ii) release one or more microbubbles (one ultrasound contrast agent); and (iii) release one or more therapeutic agents, in which the microbubbles burst upon receiving the energy thereby disrupting the target and surrounding extracellular matrix (ECM) and allowing for the one or more therapeutic agents to be delivered within the target.

In some embodiments, the one or more microbubbles are configured and arranged to contain the one or more therapeutic agents, wherein the one or more microbubbles burst upon receiving the energy thereby releasing the one or more therapeutic agents within the target.

In another embodiment, the device further comprises a configuration and arrangement to (iv) release one or more contrast agents.

In one embodiment, the ultrasound comprises high intensity focused ultrasound (HIFU). In another embodiment, the ultrasound comprises histotripsy.

Another aspect of the present disclosure provides a method for the delivery of a drug, protein, nucleic acid or gene to a target, the method comprising using an ultrasound energy delivery system as provided herein, the ultrasound energy delivery system configured and arranged to produce an energy output; supplying one or more microbubbles and one or more therapeutic agents, wherein the microbubbles burst upon receiving the energy thereby disrupting the target and surrounding extracellular matrix (ECM) and allowing for the one or more therapeutic agents to be delivered within the target thereby treating the target.

In some embodiments, the one or more microbubbles are configured and arranged to contain the one or more therapeutic agents, wherein the one or more microbubbles burst upon receiving the energy thereby releasing the one or more therapeutic agents within the target.

In another embodiment, the method further comprises for the release of one or more contrast agents.

In another embodiment, the one or more therapeutic agents are selected from the group consisting of a cytokines, chemokines, and other biologic proteins (e.g., IL-12 and the like), oncolytic viruses, CAR-T cells, TILs, other cells, sub-cellular vesicles including exosomes, cDNA, mRNA, self-replicating RNA, proteins, antibodies, single chain antibodies, nanobodies, phage, immuno-suppressants, anti-inflammatoirenti-proliferatives, anti-migratory agents, anti-fibrotic agents, pro-apoptotics, vasodilators, calcium channel blockers, anti-neoplastics, anti-cancer agents, anti-thrombotic agents, anti-platelet agents, IIb/IIIa agents, antiviral agents, mTOR (mammalian target of rapamycin) inhibitors, and combinations thereof.

In one embodiment, the one or more therapeutic agents comprise a cytokine. In one embodiment, the cytokine comprises IL-12, IL-15, or fusion proteins of cytokines.

In one embodiment, the invention provides a catheter. The catheter includes an elongated hollow tube, a first lumen in the elongated hollow tube, a second lumen in the elongated hollow tube, a transducer positioned within the elongated hollow tube and adjacent to the first lumen or the second lumen, and a needle. The ultrasonic transducer configured to emit ultrasound waves through the lumen to a target and the needle is positioned within the elongated hollow tube and configured to extend from the first lumen or the second lumen to enter the target to deliver a therapy to the target.

In another embodiment, the disclosure provides a method of treating a malignant tumor. The method includes inserting the catheter described above within a subject and toward the malignant tumor, activating the transducer to deliver energy through the first lumen to the malignant tumor that results in acoustic peak negative pressure applied to the malignant tumor within a range of 10 MPa to 40 MPa, and activating the needle to extend from the second lumen to enter the malignant tumor to deliver a therapeutically effective amount of a pharmaceutical composition.

In another embodiment, the disclosure provides a system for the treatment of a target. The system includes an ultrasound energy source and a device coupled to the ultrasound energy source. The device is configured and arranged to direct the ultrasound energy to a target, release one or more microbubbles, and release one or more therapeutic agents, in which the microbubbles burst upon receiving the ultrasound energy thereby disrupting the target and surrounding extracellular matrix (ECM) of the target and allowing for the one or more therapeutic agents to be delivered within the target.

In another embodiment, the disclosure provides a method for the delivery of a drug to a target. The method includes inserting a catheter within a subject and toward the target, the catheter including the device described above, generating an ultrasound energy output near the target, supplying one or more microbubbles near the target, and supplying one or more therapeutic agents through the catheter, wherein the microbubbles burst upon receiving the ultrasound energy thereby disrupting the target and surrounding extracellular matrix (ECM) and allowing for the one or more therapeutic agents to be delivered within the target thereby treating the target.

Other aspects of the invention will become apparent by consideration of the detailed description and accompanying drawings.

### BRIEF DESCRIPTION OF THE DRAWINGS

FIG. 1A is a schematic showing a system comprising an ultrasound energy source for the delivery of a therapeutic agent to a target tissue in accordance with some embodiments.

FIG. 1B is a schematic showing a system comprising an ultrasound energy source for the delivery of a therapeutic agent to a target tissue in accordance with some embodiments.

FIG. 2A is a transducer example of the system of FIGS. 1A and 1B in accordance with some embodiments.

FIG. 2B is a graph depicting a sensitivity of the transducer of FIG. 2A in accordance with some embodiments.

FIG. 2C is an image of a sonoporation drug delivery test set-up for the transducer of FIG. 2A in accordance with some embodiments.

FIG. 2D are fluorescence images of GFP+ sonoporation-treated cells and Luciferase activities of negative control cells and sonoporation-treated cells treated via the transducer of FIG. 2A in accordance with some embodiments.

FIG. 3A is a tubular HIFU transducer example in accordance with some embodiments.

FIG. 3B is a schematic view of the transduce of FIG. 3A in accordance with some embodiments.

FIG. 3C is a simulated acoustic field profile generated by the transducer of FIG. 3A in accordance with some embodiments.

FIG. 3D is a measured acoustic profile in both a side-viewing and a forward-viewing direction generated by the transducer of FIG. 3A in accordance with some embodiments.

FIG. 4A is a miniaturized HIFU transducer in accordance with some embodiments.

FIG. 4B is a graph depicting an acoustic pressure output of the transducer of FIG. 4A in accordance with some embodiments.

FIG. 4C is a resultant image of a bubble cloud generation test performed by the transducer of FIG. 4A in accordance with some embodiments.

FIG. 5A illustrates a plurality of views of a HIFU transducer in accordance with some embodiments.

FIG. 5B is a simulated acoustic pressure field profile of the transducer of FIG. 5A under a frequency operation of 3.5 MHz in accordance with some embodiments.

FIG. 5C is a diagram of a set up for cavitation generation using the transducer of FIG. 5A in accordance with some embodiments.

FIG. 5D is a diagram of a set up for cavitation generation using the transducer of FIG. 5A in accordance with some embodiments.

FIG. 6A is a pair of plots depicting a tumor volume size and survival rate of mice treated with TAVO and control plasmid in accordance with some embodiments.

FIG. 6B is a pair of plots depicting a tumor volume fold change for treated and untreated mice in accordance with some embodiments.

FIG. 6C depicts a t-SNE plot of cells classified into cell types for all samples or divided by treatment group of FIGS. 6A and 6B in accordance with some embodiments.

FIG. 6D depicts quantification plots of a frequency of each clone for each treatment group of FIGS. 6A and 6B in accordance with some embodiments.

FIG. 6E depicts quantification plots of an activation signature score across all t-cells in each treatment group of FIGS. 6A and 6B in accordance with some embodiments.

FIG. 7A is a circus plot depicting receptor-ligand interactions between receptors on CD8 T cells and ligands on macrophages of the treatment groups of FIGS. 6A and 6B in accordance with some embodiments.

FIG. 7B is a plot depicting 50 gene CXCR3 gene signature scores quantified across all cells in accordance with some embodiments.

FIG. 8A is a tumor curve from a combination therapy performed with the transducer of FIGS. 4A and 5A in accordance with some embodiments.

FIG. 8B is a tumor curve from a combination therapy performed with the transducer of FIGS. 4A and 5A in accordance with some embodiments.

FIG. 8C is a tumor curve from a combination therapy performed with the transducer of FIGS. 4A and 5A in accordance with some embodiments.

FIG. 8D is a tumor curve from a combination therapy performed with the transducer of FIGS. 4A and 5A in accordance with some embodiments.

FIG. 9 is a pair of images depicting a baseline image and a post-cycle image of lung metastases in accordance with some embodiments.

FIG. 10 depicts a plurality of images of fibrotic tumor stromal in breast cancer in accordance with some embodiments.

FIG. 11 depicts images of drug distribution after intratumoral injection of PV-10 drug in accordance with some embodiments.

FIG. 12 are images depicting how tumors are treated with different HIFU protocols in accordance with some embodiments.

FIG. 13 is a histological graph and plot depicting HIFU treatment data and control (no treatment) data of T cell infiltration of tumors in accordance with some embodiments.

FIG. 14 includes graphs depicting a combination of M-HIFU and PD-L1 treatment in accordance with some embodiments.

FIG. 15 includes plots depicting results of combination treatment of IT-IL-12 gene therapy with M-HIFU in accordance with some embodiments.

FIG. 16A is a system including the transducer of FIG. 2A for treatment of metastatic breast cancer in accordance with some embodiments.

FIG. 16B is a system including the transducer of FIG. 2A for treatment of metastatic breast cancer in accordance with some embodiments.

FIG. 17 are graphs depicting ESR1 mutant expression conferring constitutive estrogen signaling and enhanced growth in pre-malignant murine breast epithelial cells in accordance with some embodiments.

FIG. 18 is another example of a transducer of the system of FIGS. 1A and 1B in accordance with some embodiments.

FIG. 19 is a table of acoustic properties of a cell culture plate for use with the transducer of FIG. 18 in accordance with some embodiments.

FIG. 20 is a system for testing functionality of the transducer of FIG. 18 in accordance with some embodiments.

FIG. 21 is a graph depicting the acoustic pressure output of the transducer of FIG. 18 in accordance with some embodiments.

FIG. 22 is an acoustic pressure field of a simulated 800 kHz ultrasound beam for testing functionality of the transducer of FIG. 18 in accordance with some embodiments.

FIG. 23 is a table depicting luciferase activity for the control group and experiment group for testing functionality of the transducer of FIG. 18 in accordance with some embodiments.

FIG. 24 is a series of graphss depicting luciferase activity for sonoporation tests under various sonication parameters for testing functionality of the transducer of FIG. 18 in accordance with some embodiments.

FIG. 25 is a series of tables depicting growth suppression of local and distant tumors and enhanced tumor antigen-specific cellular immune responses in accordance with some embodiments.

FIG. 26 is a series of data plots regarding enhanced intratumoral infiltration by M-HIFU treatment in accordance with some embodiments.

FIG. 27A is a series of plots of GO enrichment analyses and KEGG pathway analyses of DEGs in macrophages after M-HIFU treatment in accordance with some embodiments.

FIG. 27B is a series of plots of GO enrichment analyses and KEGG pathway analyses of DEGs in macrophages after M-HIFU treatment in accordance with some embodiments.

FIG. 28 is a series of plots depicting enhanced expression of immune checkpoint molecules by tumor-infiltrating immune cells after M-HIFU treatment in accordance with some embodiments.

FIG. 29 is a series of plots depicting M-HIFU and PD-L1 blockade synergize to reject local tumor. in accordance with some embodiments.

FIG. 30 is a series of plots depicting the combination of M-HIFU and anti-PD-L1 antibody induces upregulation of unique DEGs in tumor-infiltrating CD8 T cells and macrophages luciferase activity in accordance with some embodiments.

FIG. 31 is a series of plots depicting results of the combination of M-HIFU and PD-1/PD-L1 blockades in accordance with some embodiments.

FIG. 32 illustrates an example sonoporation transducer used in Example 7.

FIG. 33 is a table showing test conditions for test groups and control groups used in Example 7.

FIGS. 34-37 illustrate data from experimental test results for Example 7.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of”′ (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like.

As is known in the art, a cancer is generally considered as uncontrolled cell growth. The methods of the present invention can be used to treat any cancer, and any metastases thereof, including, but not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma.

As used herein, the term “subject” and “patient” are used interchangeably herein and refer to both human and nonhuman animals. The term “nonhuman animals” of the disclosure includes all vertebrates, e g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e, living organism, such as a patient).

As used herein, “therapeutic agent” includes any molecular species, and/or biologic agent that is either therapeutic as it is introduced to the subject under treatment, becomes therapeutic after being introduced to the subject under treatment, for example by way of reaction with a native or non-native substance or condition, or any other introduced substance. Examples of native conditions include pH (e.g., acidity), chemicals, temperature, salinity, osmolality, and conductivity; with non-native conditions including those such as magnetic fields, electromagnetic fields (such as radiofrequency and microwave), and ultrasound. In the present disclosure, the chemical name of any of the therapeutic agents is used to refer to the compound itself and to pro-drugs (precursor substances that are converted into an active form of the compound in the body), and/or pharmaceutical derivatives, analogues, or metabolites thereof (bio-active compound to which the compound converts within the body directly or upon introduction of other agents or conditions (e.g., enzymatic, chemical, energy), or environment (e.g., pH).

The scope of the present disclosure includes the use of any therapeutic agent whose medicinal effectiveness may be enhanced by the use of ultrasonic energy, as described herein. For the purposes of illustration, a number of therapeutic agent classes are identified in order to convey an understanding of the present disclosure. These classes of agents and the specifically listed agents are not intended to limit the scope or practice of the invention in any way; the scope of the present disclosure includes any therapeutic agent that may be considered beneficial in the treatment of a patient. Further, these agents may be delivered/administered by any appropriate modality. As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

In some embodiments, examples of therapeutic agents may include cytokines, chemokines, and other biologic proteins (e.g, IL-12 and the like), oncolytic viruses, CAR-T cells, TILs, cDNA, mRNA, self-replicating RNA, proteins, immuno-suppressants, anti-inflammatories anti-proliferatives, anti-migratory agents, anti-fibrotic agents, pro-apoptotics, vasodilators, calcium channel blockers, anti-neoplastics, anti-cancer agents, antibodies, anti-thrombotic agents, anti-platelet agents, IIb/IIIa agents, antiviral agents, mTOR (mammalian target of rapamycin) inhibitors, non-immunosuppressant agents, and combinations thereof.

Unless otherwise defined, all technical terms used herein have the same meaning as commonly understood by one of ordinary skill in the art to which this disclosure belongs.

Aspects of the present disclosure combine a source of energy with microbubbles and one or more therapeutic agents to treat selected regions (e.g., a tumor) of a subject. As used herein the terms “selected regions,”, “treatment site,” “desired target,” and “target region(s)” are used interchangeably and refer to an area within a subject where targeted treatment is desired. The treatment site may include tissues associated with bodily lumens, organs, or localized tumors. In one embodiment, the present devices and methods reduce the formation or progression of a tumor or hyperplastic growth. A “lumen” may be any blood vessel in the subject's vasculature, including veins, arteries, aorta, and particularly including coronary and peripheral arteries, as well as previously implanted grafts, shunts, fistulas, and the like. In other embodiments, systems and methods described herein may also be applied to other body lumens, such as the biliary duct, which are subject to excessive neoplastic cell growth. Examples of internal corporeal tissue and organ applications include various organs, nerves, glands, ducts, and the like.

According to one aspect of the present disclosure, a system for the treatment of a tumor is provided. In such aspects, the system comprises: (a) an ultrasound energy source; (b) a device coupled to the energy source and configured and arranged to: (i) direct the energy to a desired location; (ii) release one or more microbubbles; and (iii) release one or more therapeutic agents. As used herein, the term “microbubbles”, a type of ultrasound contrast agent, also referred to herein as “cavitating bubbles” or “cavitation bubbles,” are tiny, gas-filled bubbles that can be injected into a subject where they remain inactive unless stimulated by energy generated by an energy source. In practice, the energy source directs the energy at the microbubbles causing them to vibrate and rupture thereby disrupting the surrounding tissue (e.g., ECM, tumor, etc.) and allowing for the one or more therapeutic agents to be delivered within the tumor.

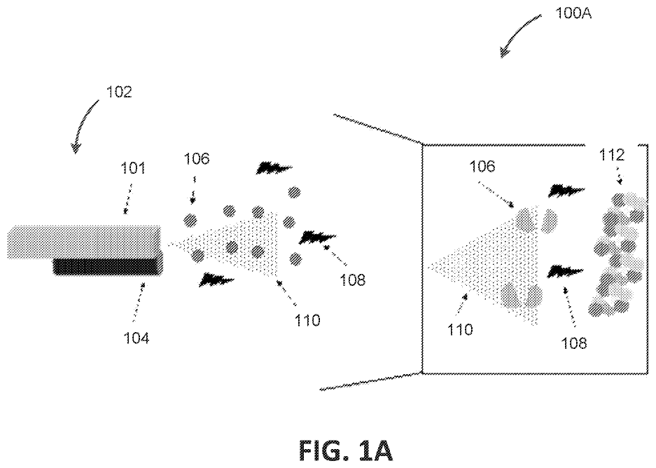

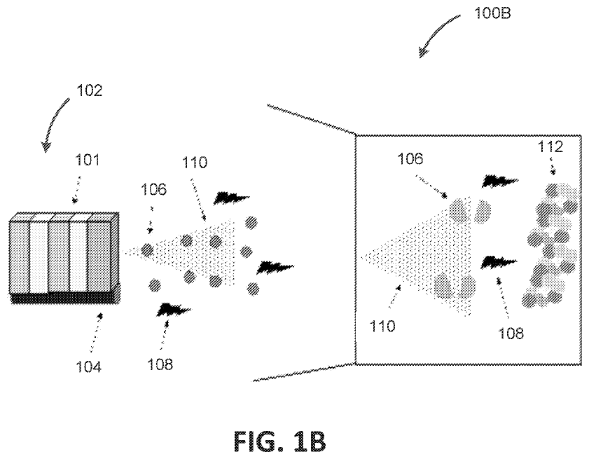

An example of a system for the delivery of energy from an ultrasound energy source to microbubbles (and, in some embodiments, for the delivery of a therapeutic agent to a desired target (e.g., a tumor)) according to some embodiments of the present disclosure are shown in FIGS. 1A and 1B. FIGS. 1A and 1B each illustrate an example of a system 100A, 100B for the delivery of energy from an ultrasound energy source to microbubbles to a desired target according to some embodiments. According to the example illustrated in FIG. 1A, the system 100 includes a device 102 comprising a simple or single-source ultrasound energy source 101. FIG. 1B illustrates another example where the device 102 comprises an ultrasound energy source 101 comprising a composite ultrasound device (for example, a probe or transducer).

It should be understood that, although FIGS. 1A and 1B illustrate different types of ultrasound energy sources 101, any source of ultrasound suitable for therapeutic applications may be used in the systems and methods provided herein. Suitable examples include, but are not limited to, HIFU, histotripsy, and the like.

In any instance, the device 102 comprises one or more adjacent tubes 104 for the release of microbubbles 106 and/or therapeutic agents 108. Once released from the device 102, energy 110 is released from the energy source 101 within the device 102 and delivered to the microbubbles 106. The microbubbles 106 are then activated which allows for the disruption of the target tissue 112 and/or subsequent delivery of the one or more therapeutic agents 108 to the target tissue 112. It should be understood that, although the examples illustrated in FIGS. 1A and 1B depict the microbubbles and therapeutic agent(s) being delivered in a longitudinal direction, in some embodiments the microbubbles and/or therapeutic agent(s) may be delivered alternatively or additionally in one or more other directions (for example, in a transverse direction).

In some embodiments, the microbubbles and therapeutic agents are delivered to the subject simultaneously. In other embodiments, the microbubbles are delivered to the subject prior to the delivery of the therapeutic agent(s). In yet other embodiments, the microbubbles are delivered to the subject after the therapeutic agent(s).

In yet other embodiments, the therapeutic agent(s) are loaded within the microbubbles where they can be ruptured or “activated” to release their content by slight interaction with the applied energy. By “loading” these microbubbles with a specific therapeutic agent(s) and then activating the microbubbles to release their contents at the target tissue, side effects of the therapeutic agent(s) can be limited by delivering it only to the needed site (or substantially so). In such embodiments, the microbubble may comprise a tiny, gas-filled lipid, or fat. As a result, there is a high concentration of the therapeutic agent(s) in the target tissue (e.g., tumor) for destruction of such. Despite the presence of the drug in the blood, however, the other tissues in the body are spared because the drug is attached to the microbubbles and is inactive.

In another embodiment, the system further provides for visualizing the target tissue. Gas filled microbubbles (e.g., on the order of microns in size/diameter) can be visualized or imaged by ultrasound energy application. For example, in one embodiment, the system may utilize an energy source that directs the energy at the microbubbles, causing them to return a unique echo within the bloodstream that produces a dramatic distinction, or high “contrast,” between blood vessels and surrounding tissue, thus enabling clinicians to visualize the target area. In other embodiments, visualization may be accomplished by the administration of a contrast agent. In either case, the contrast properties of the agents and/or microbubbles allow for the visualization of the target tissue.

The systems provided herein can be used in numerous methods for treating a disease, such as cancer. In another aspect, the present disclosure provides a method for the delivery of a drug to a tumor, the method comprising using an energy delivery system as provided herein, the energy delivery system configured and arranged to produce an energy output; supplying one or more microbubbles and one or more therapeutic agents, wherein the microbubbles burst upon receiving the energy thereby disrupting the tumor and surrounding extracellular matrix (“ECM”) and allowing for the one or more therapeutic agents to be delivered within the tumor thereby treating the tumor.

In some embodiments, the one or more microbubbles are configured and arranged to contain the one or more therapeutic agents, wherein the one or more microbubbles burst upon receiving the energy thereby releasing the one or more therapeutic agents within the tumor.

In some embodiments, the method comprises introducing anti-cancer therapeutic agents for promoting intracellular activation by exposing the vessel wall cells to an energy to cause passage of these drugs into the target tissue.

For example, suitable drugs within the scope of the present disclosure include, but are not limited to, the following: Adriamycin PFS Injection (Pharmacia & Upjohn); Adriamycin RDF for Injection (Pharmacia & Upjohn); Alkeran for Injection (Glaxo Wellcome Oncology/HIV); Aredia for Injection (Novartis); BiCNU (Bristol-Myers Squibb Oncology/Immunology); Blenoxane (Bristol-Myers Squibb Oncology/-Immunology); Camptosar Injection (Pharmacia & Upjohn); Celestone Soluspan Suspension (Schering); Cerubidine for Injection (Bedford); Cosmegen for Injection (Merck); Cytoxan for Injection (Bristol-Myers Squibb Oncology/Immunology); DaunoXome (NeXstar); Depo-Provera Sterile Aqueous Suspension (Pharmacia & Upjohn); Didronel I.V. Infusion (MGI): Doxil Injection (Sequus): Doxorubicin Hydrochloride for Injection, USP (Astra); Doxorubicin Hydrochloride Injection, USP (ASTRA); DTIC-Dome (Bayer); Elspar (Merck); Epogen for Injection (Amgen); Ethyol for Injection (Alza); Etopophos for Injection (Bristol-Myers Squibb Oncology/Immunology); Etoposide Injection (Astra); Fludara for Injection (Berlex): Fluorouracil Injection (Roche Laboratories); Gemzar for Injection (Lilly); Hycamtin for Injection (SmithKline Beecham); Idamycin for Injection (Pharmacia & Upjohn); Ifex for Injection (Bristol-Myers Squibb Oncology/Immunology); Intron A for Injection (Schering); Kytril Injection (SmithKline Beecham); Leucovorin Calcium for Injection (Immunex); Leucovorin Calcium for Injection, Wellcovorin Brand (Glaxo Welcome Oncology/HIV); Leukine (Immunex); Leustatin Injection (Ortho Biotech); Lupron Injection (Tap); Mesnex Injection (Bristol-Myers Squibb Oncology/Immunology); Methotrexate Sodium Tablets, Injection, for Injection and LPF Injection (Immunex); Mithracin for Intravenous Use (Bayer); Mustargen for Injection (Bristol-Myers Squibb Oncology/Immunology); Mutamycin for Injection (Bristol-Myers Squibb Oncology/-Immunology), Navelbine Injection (Glaxo Wellcome Oncology/HIV); Neupogen for Injection (Amgen); Nipent for Injection (SuperGen); Novantrone for Injection (Immunex); Oncaspar (Rhone-Poulenc Rorer); Oncovin Solution Vials & Hyporets (Lilly); Paraplatin for Injection (Bristol-Myers Squibb Oncology/Immunology), Photofrin for Injection (Sanofi); Platinol for Injection (Bristol-Myers Squibb Oncology/Immunology); Platinol-AQ Injection (Bristol-Myers Squibb Oncology/Immunology); Procrit for Injection (Ortho Biotech); Proleukin for Injection (Chiron Therapeutics); Roferon-A Injection (Roche Laboratories); Rubex for Injection (Bristol-Myers Squibb Oncology/Immunology); Sandostatin Injection (Novartis); Sterile FUDR (Roche Laboratories); Taxol Injection (Bristol-Myers Squibb Oncology/Immunology); Taxol Abraxane-ABI-007 (Abraxis Bioscience); Taxotere for Injection Concentrate (Rhone-Poulenc Rorer); TheraCys BCG Live (Intravesical) (Pasteur Merieux Connaught); Thioplex for Injection (Immunex); Tice BCG Vaccine, USP (Organon); Velban Vials (Lilly); Vumon for Injection (Bristol-Myers Squibb Oncology/Immunology); Zinecard for Injection (Pharmacia & Upjohn); Zofran Injection (Glaxo Wellcome Oncology/HIV); Zofran Injection Premixed (Glaxo Wellcome Oncology/HIV); Zoladex (Zeneca).

Other classes of drugs within the scope of the present disclosure include alkylating agents which target DNA and are cytoxic, nutagenic, and carcinogenic. All alkylating agents produce alkylation through the formation of intermediate. Alkylating agents impair cell function by transferring alkyl groups to amino, cartoryl, sulfhydryl, or phosphate groups of biologically important molecules. Such drugs include, but are not limited to, Busulfan (Myleran), Chlorambucil (Leukeran), Cyclophosphamide (Cytoxan, Neosor, Endoxus), Ifosfamide (Isophosphamide, Ifex), Melphhalan (Alkeran, Phenylalanine Mustargen, L-Pam, L-Sarcolysin), Nitrogen Mustargen (Mechlorethamine, Mustargen, HIV2), Nitrosonceas (Carmustine CBCNV, Bischlorethyl, Nitrosourea), Lomustine (CCNV, Cyclohexyl Chlorethyl Nitrosouren, CeeNV), semustine (methyl-CCNV) and Streptozocin (Strephozotocin), Streptozocin (Streptozoticin, Zanosan), Thiotepa (Theo-TEPA, and Triethylenethrophosphoranide).

Agents with alkylator activity include a group of compounds that include heavy metal alkylators (platinum complexes) that act predominantly by covalent bonding and “non-classic alkylating agents” are also within the scope of the present disclosure. Such agents typically contain a chloromethyl groups and an important N-methyl group. Such other agents include, but are not limited to, Amsacrine (m-AMSA, msa, Acridinylanisidiale, 4′-) (9-acridinylamins) methanesulfin-m-anesidide, Carboplatin (Paraplatin, Carboplatinum, CBDCA), Cisplatin (Cesplatinum), Dacabazine (DTIC, DIC dimethyltricizenormidazoleconboxamide), Hexamethylmelanine (HMM, Altretanine, Hexylin) and Procarbazine (Matulane, Natulanan).

Antimetabolite drugs are also included within the scope of the present disclosure, and include, but are not limited to, examples such as Azacitidine (5-azacylidine, ladakamycin) Cladribine (2-CdA, CdA, 2-chloro-2-deoxyadenosine) Cytarabine (Cytosine Arabinoside, Cytosar, Tarabine), Fludarabine (2-fluoroadenine arabinoside-5-phosphate, fludara). Fluorouracil (5-FV, Adrucil, Efuctex) Hydroxyurea (hydroxycarbamide, Hydrea), Leucovorin (Leucovorin Calcium), Mercaptopurine (G-MP, Purinethol), Methotrexate (Amethopterin), Mitoguazone (Methyl-GAG), Pentostatin (2′-deorycoformycin) and Thioguanine (6-TG, aminopurine-6-thiol-hemihydrate).

Antitumor antibiotics commonly interfere with DNA through intercalation, whereby the drug inserts itself between DNA base pairs. Introduction of ultrasound enhances this interference. Such drugs include, but are not limited to, Actinomycin DC Cosmegen, Dactinomycin), Bleomycin (Blenoxane) Daunoxubibin (rubidomycin), Doxorubicin (Adriamycin, Hydroxydaunorubicin, hydroxydaunomycin, Rubex), Idarubicin (44-demethylorydan norubicin, Idamycin), Mithramycin (Mithracin, Plicamycin), Milomycin C and Mitorantione (Novantrone).

Plant alkaloids bind to microtubular proteins thus inhibiting microtubule assembly; and energy such as laser or ultrasound may enhance such binding. Such alkaloids include, but are not limited to, Etoposide, Paclitaxel (Taxol), Treniposide, Vinblastine (Velban, Velsar, Alkaban), Vincristine (Oncovin, Vincasar, Leurocristine) and Vindesine (Eldisine).

Hormonal agents include steroids and related agonists and antagonists, such as, but not limited to, adrenocorticosteroids, adrenocorticosteroid inhibitors, mitolane, androzens, antiandiozens, antiestrogens, estrogens, LHRH agonists, progesterones.

Antiangiogenesis agents include, but are not limited to, Fumagillin-derivative TNP-470, Platelet Factor 4, Interleukin-12, Metalloproteinase inhibitor Batimastat, Carboryaminatriarzole, Thalidomide, Interferon Alfa-2a, Linomide and Sulfated Polysaccharide Tecogalan (DS-4152).

Specific examples of therapeutic agents that may be used in various embodiments include, but are not limited to: mycophenolic acid, mycophenolic acid derivatives (e.g., 2-methoxymethyl derivative and 2-methyl derivative), VX-148, VX-944, mycophenolate mofetil, mizoribine, methylprednisolone, dexamethasone, CERTICAN™ (e.g., everolimus, RAD), rapamycin, ABT-773 (Abbot Labs), ABT-797 (Abbot Labs), TRIPTOLIDE™, METHOTREXATE™, phenylalkylamines (e.g., verapamil), benzothiazepines (e.g., diltiazem), 1,4-dihydropyridines (e.g., benidipine, nifedipine, nicarrdipine, isradipine, felodipine, amlodipine, nilvadipine, nisoldipine, manidipine, nitrendipine, barnidipine (HYPOCA™)), ASCOMYCIN™, WORTMANNIN™, LY294002, CAMPTOTHECIN™, flavopiridol, isoquinoline, HA-1077 (1-(5-isoquinolinesulfonyl)-homopiperazine hydrochloride), TAS-301 (3-bis(4-methoxyphenyl)methylene-2-indolinone), TOPOTECAN™, hydroxyurea, TACROLIMUS™ (FK 506), cyclophosphamide, cyclosporine, daclizumab, azathioprine, prednisone, diferuloymethane, diferuloylmethane, diferulylmethane, GEMCITABINE™, cilostazol (PLETAL™), tranilast, enalapril, quercetin, suramin, estradiol, cycloheximide, tiazofurin, zafurin, AP23573, rapamycin derivatives, non-immunosuppressive analogues of rapamycin (e.g., rapalog, AP21967, derivatives' of rapalog), CCI-779 (an analogue of rapamycin available from Wyeth), sodium mycophernolic acid, benidipine hydrochloride, sirolimus, rapamine, metabolites, derivatives, and/or combinations thereof.

In other embodiments, therapeutic agents may include cytokines, including but not limited to, IL-2, TNF-, IL-12 and the like.

The systems and devices of the present disclosure may be configured to release or make available the therapeutic agent at one or more treatment phases, the one or more phases having similar or different performance (e.g., delivery) profiles. The therapeutic agent may be made available to the tissue at amounts which may be sustainable, intermittent, or continuous; in one or more phases and/or rates of delivery. Any one of the at least one therapeutic agents may perform one or more functions, including preventing or reducing proliferative activity, reducing or inhibiting tumor formation and/or growth or the like.

The total amount of therapeutic agent made available to the tissue depends in part on the level and amount of desired therapeutic result. The therapeutic agent may be made available at one or more phases, each phase having similar or different release rate and duration as the other phases. The release rate may be pre-defined. In an embodiment, the rate of release may provide a sustainable level of therapeutic agent to the treatment site. In another embodiment, the rate of release is substantially constant. The rate may decrease and/or increase as desired.

These therapeutic agents may be provided and or delivered to the subject or target in any conventional therapeutic form or formulation, such as, merely by way of example: liquid, powder, particle, microbubbles, microspheres, nanospheres, liposomes and/or combinations thereof.

Some embodiments of the present disclosure may also include delivering at least one therapeutic agent(s) and/or optional compound within the subject or target concurrently with or subsequent to an interventional treatment. More specifically, the therapeutic agent may be delivered to a targeted site that includes the treatment site concurrently with or subsequent to the interventional treatment. By way of example: (a) A therapeutic agent may be delivered to the treatment site as a stand-alone therapy in treatment of a tumor, without any other contemporaneous treatment such as provided by a physical or mechanical intervention; (b) A therapeutic agent may be delivered to the treatment site as the only therapy in treatment of a disease (e.g., a tumor); (c) A therapeutic agent may be delivered to the treatment site following any suitable interventional procedure; (d) A therapeutic agent may be delivered to the treatment site before an interventional procedure, during, after an interventional procedure, or combinations thereof.

The therapeutic agent(s) may be made available to the treatment site at amounts which may be sustainable, intermittent, or continuous; at one or more phases; and/or rates of delivery.

A micro-transducer based catheter is disclosed herein that uses different sonic wave energy levels to a) overcome the current anatomic barriers of HIFU therapies (as described above) by directly impacting tumor and ablating primary and metastatic lesions, b) disrupt tumor extracellular matrix with or without contrast agents to facilitate intratumoral delivery of therapeutics, and c) sonoporate (pulsed interruption of tumor cell membrane for enhanced permeability) to allow nucleotide medicine such as mRNA to enter live tumor cells. The micro-transducer based catheter is configured to directly deliver a therapeutic into a tumor using an image-guided microtransducer HIFU catheter, using currently available imaging equipment and technologies. The device is configured to deliver a range of high-pressure acoustic bursts of focused ultrasound to create intratumoral acoustic cavitation, in which the expansion and collapse of microbubbles releases high-pressure cavitation energy that destroys the physical barriers in tumors and cancer cells by membrane disruption, allowing a range of ablation and/or effective intratumoral retention and uptake of therapeutics.

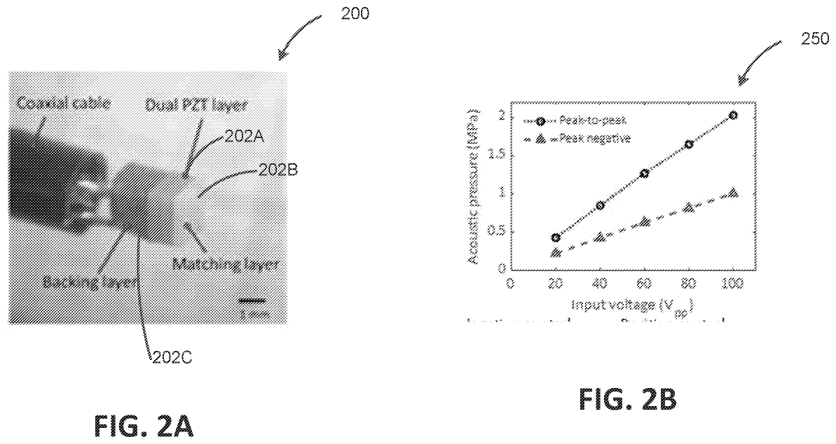

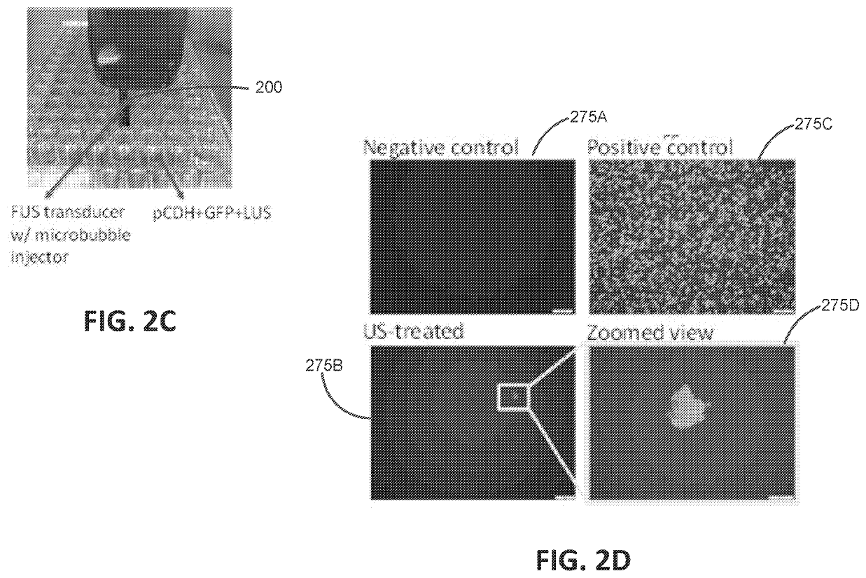

FIG. 2A illustrates a forward-looking ultrasound (US) transducer 200 in accordance with some embodiments. The transducer 200 comprises a plurality of layers. For example, as illustrated, the transducer 200 includes a double-layered PZT-5A layer 202A, a matching (Al₂O₃/epoxy) layer 202B, and a backing (air bubble/epoxy) layer 202C. The aperture area is approximately 2×2 mm², and the operation frequency is configured for approximately 0.8 MHz. Additionally, in other embodiments, the piezoelectric material can vary, the aperture area may vary in size and the operation frequency also can vary.

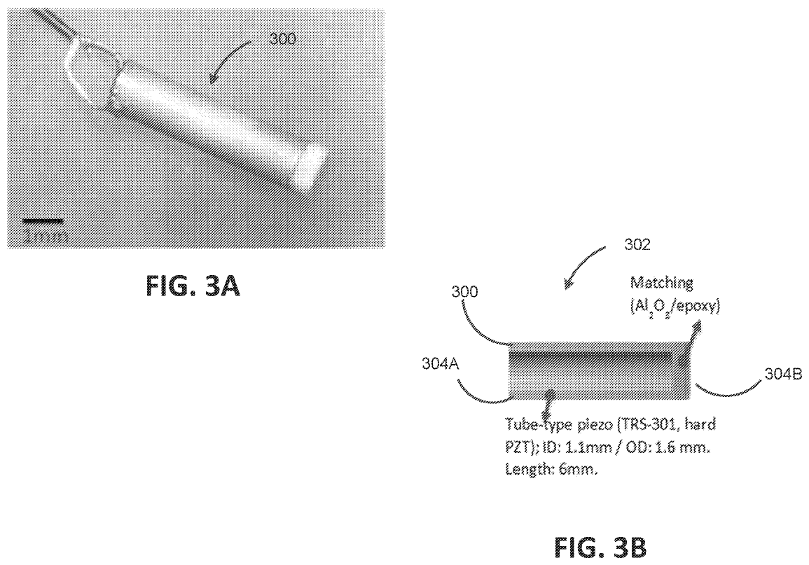

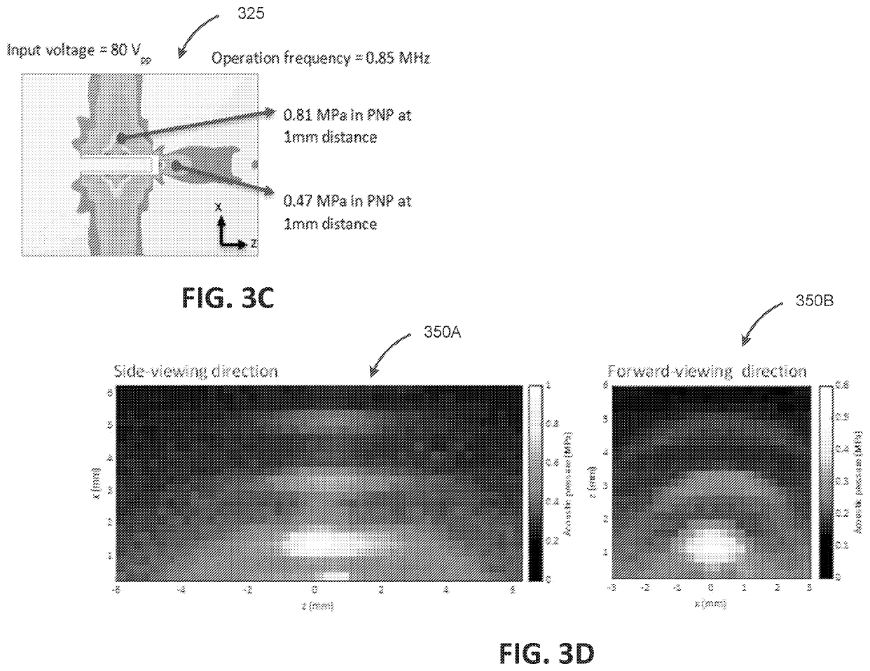

In some embodiments, the US transducer 200 may be configured as a tubular HIFU transducer. For example, FIG. 3A depicts a tubular HIFU transducer 300 for sonoporation. FIG. 3B depicts a schematic view 302 of the transducer 300, which includes a tube-type piezo 304A and a matching layer 304B.

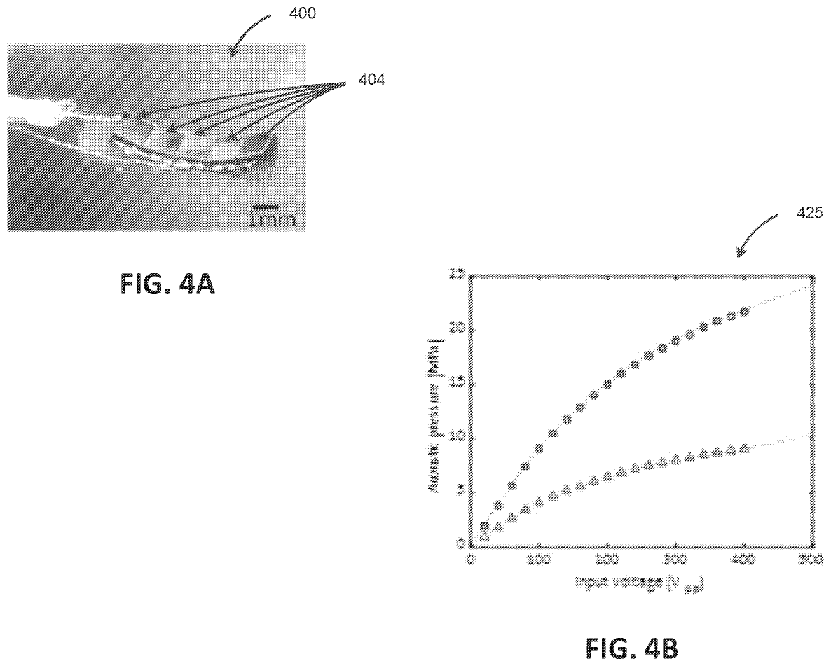

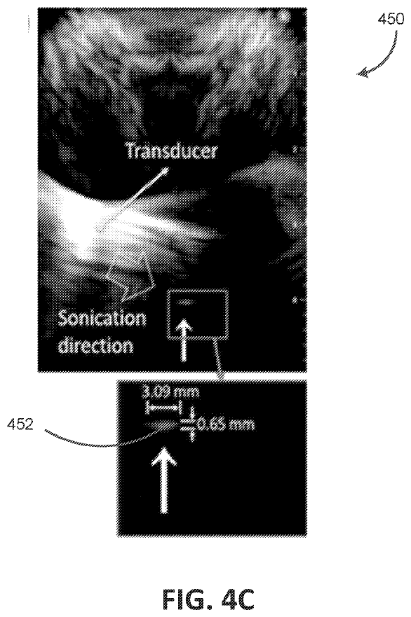

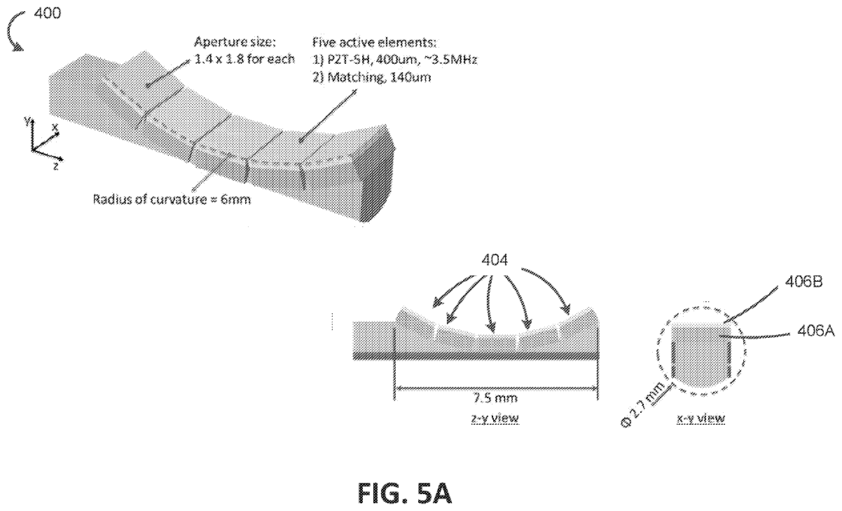

FIG. 4A depicts a miniaturized HIFU transducer 400 in accordance with some embodiments. The miniaturized HIFU transducer 400 is configured to have a center frequency of 5 MHz and, in the illustrated embodiments, includes five active elements 404 (for example, PZT-4 plates). In other embodiments, the transducer 400 may include more than five or less than five active elements 404. Additionally, in other embodiments, each element 404 may vary in size.

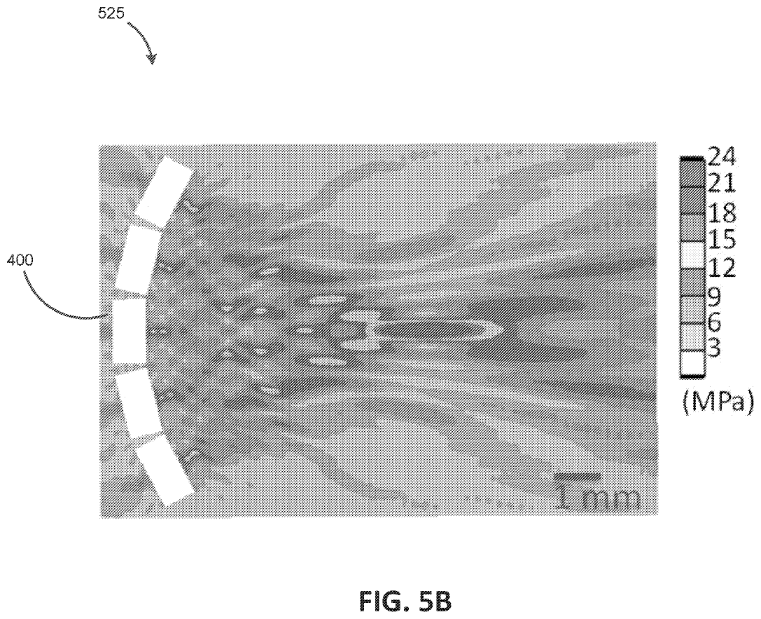

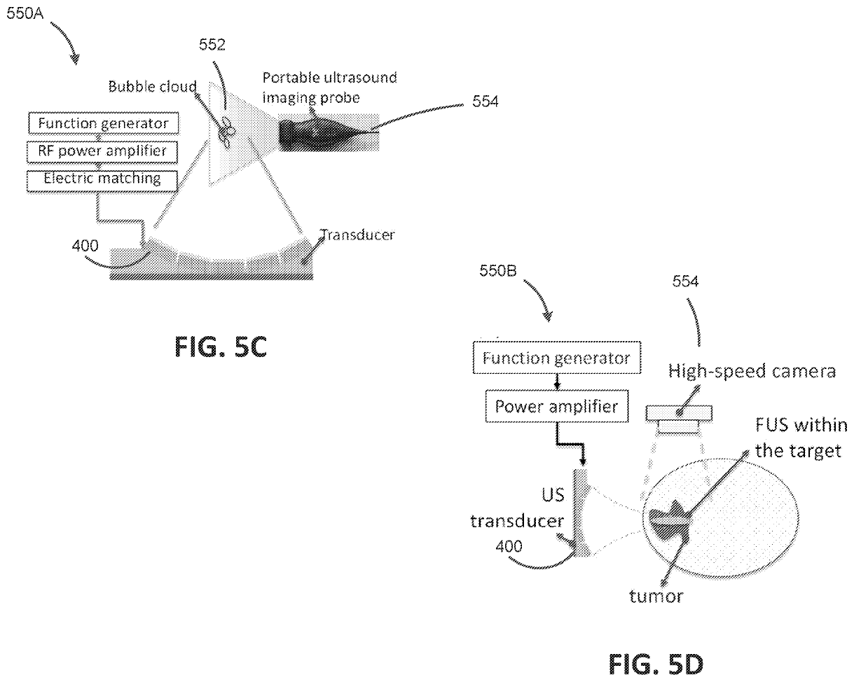

The US transducer 200 may be configured as a transcatheter HIFU device, such as those described herein (for example, the transducer 400 of FIG. 4A), and provide, for example, localized delivery of M-HIFU as well as IL-12 gene delivery into tumors (as explained in more detail below). For example, FIG. 5A shows another example embodiment of the HIFU transducer 400, which may be integrated with the hollow tube 104 of the device 102 of FIGS. 1A, 1B. For example, the transducer 400 may be integrated with a drug delivery lumen into a 6-10 Fr catheter. The transducer 400 includes a plurality of active elements 404, each of which include a plurality of layers including, for example, a PZT-5H material layer 406A and a matching layer 406B. In a non-limiting example, PZT-5H can be employed instead of PZT-4 materials since the histotripsy device 400 may operate with a relatively short pulse signal (i.e., ˜30 cycles and <1% duty cycle). The thickness of each active element 404 may range from less than about 50 μm to about 500 μm. The aperture of each element 404 is 1.4×1.8 mm² and can range from less than about 1 mm×1 mm to about 3 mm×3 mm, depending on the energy needs and application cases. In other embodiments, the transducer 400 may include more than five or less than five active elements 404. Additionally, in other embodiments, each element 404 may vary in size.

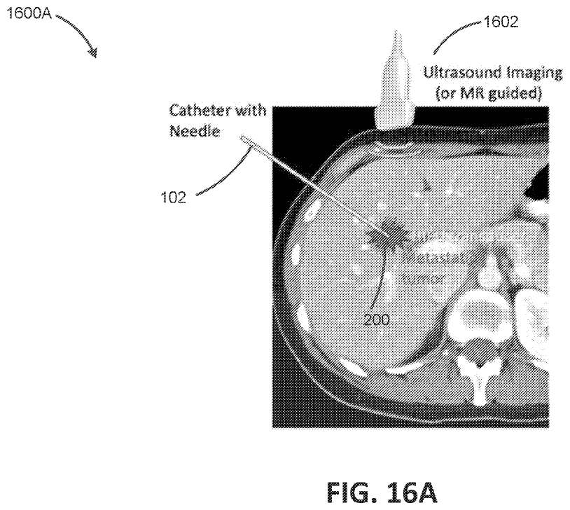

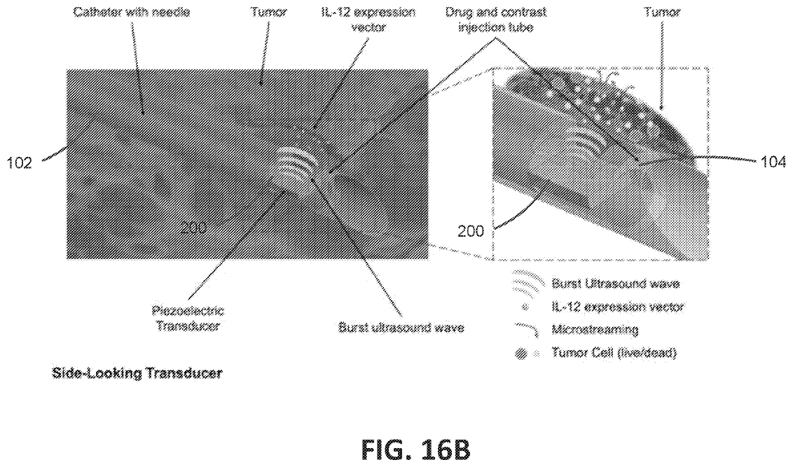

The US transducer 200, 400 is introduced through a hollow tube or catheter 104 as illustrated in FIG. 16B. The catheter 104 includes a first lumen 210 and a second lumen 212. The lumens 210, 212 may be positioned laterally along the length of the catheter or at a distal end of the catheter. For example, as illustrated in FIG. 16B, one of the lumens 210, 212 is positioned laterally on the catheter and the other one of the lumens 210, 212 is positioned at the distal end of the catheter. The US transducer 200, 400 is aligned with one of the lumens 210, 212 to emit US waves through the lumen 210, 212. The catheter 102 traverses the subject to a target site (e.g., tissue, tumor or the like) where the US transducer 200, 400 emits US waves to the target. The parameters of the US transducer 200, 400 are suitably selected for purposes of applying ablation, sonoporation, or histotripsy or HIFU procedure on the target.

### Example 1—Miniaturized HIFU Transducer|_{[HJA(1]}

A forward-looking ultrasound (US) transducer 200 as illustrated in FIG. 2A was designed for sonoporation study. The transducer 200 included a double-layered PZT-5A layer 202A, a matching (Al₂O₃/epoxy) layer 202B, and a backing (air bubble/epoxy) layer 202C. The aperture area was approximately 2×2 mm², and the operation frequency was 0.8 MHz.

FIG. 2B is a graph 250 illustrating a transmitting sensitivity of the transducer 200 as a function of the input voltage (Vpp), where the acoustic pressure was measured at the distance of 2 mm from the aperture surface of the transducer 200. FIG. 2C depicts an application of the US transducer 200 for treating HEK293 cells in the presence of plasmid DNA (pCDH-GFP-LUC, 5 μg/mL) in a 96-well plate. FIG. 2D are fluorescence images of GFP+ sonoporation-treated cells and Luciferase activities of negative control cells and sonoporation-treated cells treated via the transducer of FIG. 2A in accordance with some embodiments. As shown in images 275A-275D of FIG. 2D, focal GFP expression was observed only for the sonoporation-treated cells but not for negative control cells (without US exposure). LUC activity level was up to 3,600 units in sonoporation-treated cells (846.0+/−786.5 units), whereas that was below 170 in negative controls (76.4+/−78.6 units), indicating the successful transfection of HEK cells with pCDH-GFP-LUC by sonoporation treatment. The sonoporation may further be improved in terms of relevant ultrasound parameter selection, proper contrast agent dose, and appropriate treatment time.

A tubular HIFU transducer also was designed for sonoporation tests. FIG. 3A depicts the tubular HIFU transducer 300. FIG. 3B depicts a schematic view 302 of the transducer 300, which included a tube-type piezo 304A and a matching layer 304B. FIG. 3C illustrates a simulated acoustic field profile 325 generated by the transducer 300. The simulation was performed at an input voltage of 80 Vₚₚ and at an operation frequency of 0.85 MHz. It was found that the peak negative pressure (PNP) at both lateral and longitudinal directions at sub-MHz (e.g., 0.85 MHz) could lead to cavitation, which was necessary for the sonoporation process. A measured acoustic profile in both a side-viewing and a forward-viewing direction (profiles 350A and 350B respectively) are illustrated in FIG. 3D. Similar PNP values were obtained via measurements (approximately 0.95 MPa for the side viewing PNP and approximately 0.55 MPa for the forward viewing PNP) as those obtained from simulations (0.81 MPa and 0.47 MPa for the side and forward viewing directions respectively).

The sonoporation technique results in a gradual suppression of either tumor or cancer. In contrast, histotripsy and tissue ablation techniques aim to instantly remove or ablate malignant tumor tissue. Interstitial tissue ablation devices are relatively mature technology; this modality basically utilizes long US-wave pulses to induce thermal necrosis in the target lesion. Yet, the miniaturization of an HIFU transducer is still a challenge for catheter-directed tissue ablation. Miniaturized histotripsy transducers are very uncommon. In contrast to the typical thermal-inducing ablation technique, histotripsy utilizes very short (<1-2% duty cycle) US-wave pulses with a high rarefactional pressure output (>10 MPa). However, most histotripsy transducers operate at the outer surface of the human body. Recently, some researchers presented a relatively small size (˜5 mm) of forward-looking histotripsy transducer to treat brain tumors. However, interstitial or transcatheter HIFU capable of histotripsy is challenging because of the requirement of a high rarefactional pressure output from a small (<2 mm) HIFU aperture.

FIG. 4A depicts a miniaturized HIFU transducer 400 in accordance with some embodiments. The catheter HIFU transducer 400 is configured to have a center frequency of 5 MHz and, in the illustrated embodiments, includes five active elements 404 (for example, PZT-4 plates). The aperture of each element 404 is 1.4×1.8 mm². In other embodiments, the transducer 400 may include more than five or less than five active elements 404. Additionally, in other embodiments, each element may vary in size. FIG. 4B shows the acoustic pressure output 425 produced by the HIFU transducer 400 (line 426A indicating a peak-to-peak pressure and line 426B indicating a peak negative pressure. The quadratic regression curve indicates that a 300 Vₚₚ input would be necessary to produce a negative pressure of over 13 MPa. Next, a bubble generation test was conducted to confirm the efficacy of the HIFU transducer 400 for possible histotripsy treatment. The transducer 400 sonicated inside distilled, degassed water, and a portable ultrasound imaging probe (iQ+, Butterfly Network, Guilford, CT) was used to detect the vaporization upon the sonication (described in more detail below in regard to FIG. 5C). The whitened spot 452 in the captured image 450 of FIG. 4C shows the water vaporization induced by the HIFU transducer 400, demonstrating the capability of histotripsy treatment.

In general, a center frequency of transcatheter HIFU transducers for tissue ablation and histotripsy can range from ˜100 kHz to 7.5 MHz.

The US transducer 200 may be configured as a transcatheter HIFU device, such as those described herein (for example, the transducer 400 of FIG. 4A), and provide, for example, localized delivery of M-HIFU as well as IL-12 gene delivery into tumors (as explained in more detail below). For example, FIG. 5A shows another example embodiment of the HIFU transducer 400, which may be integrated with the tube 104 of the device 102 of FIGS. 1A, 1B. For example, the transducer 400 may be integrated with a drug delivery lumen into a 6-10 Fr catheter. The transducer 400 includes a plurality of active elements 404, each of which include a plurality of layers including, for example, a PZT-5H material layer 406A and a matching layer 406B. In a non-limiting example, PZT-5H can be employed instead of PZT-4 materials since the histotripsy device 400 may operate with a relatively short pulse signal (i.e., ˜30 cycles and <1% duty cycle) and the mechanical loss associated with transducer self-heating may not be a concern. The thickness of each active element 404 may be about 400 μm. A 3D-printed mount may serve as the confocal geometry as well as the light backing. Electrodes between individual piezoelectric plates (not shown) of the active elements 404 may be connected using, for example, coaxial cable and conductive silver epoxy.

FIG. 5B shows the acoustic pressure field profile 525 of the transducer 400 under the frequency operation of 3.5 MHz. In the linear regime, the maximum rarefactional pressure level is predicted to be over 24 MPa. The −6 dB focal zone is estimated to be about ϕ0.6×3.8 mm³. For acoustic characterization of the prototyped LM-HIFU transducer 400, a hydrophone (HNA-0400, Onda Corp., Sunnyvale, CA) was used to record the acoustic pressure field by using a 3D motion control stage.

Following the acoustic characterization, cavitation generation was using the transducer 400 was according to the set-ups 550A and 550B as shown in FIGS. 5C and 5D, the results of which are illustrated in FIG. 4C. In addition to the ultrasound imaging of the vaporized bubbles 552, a high-speed camera and an ultrasound imaging probe 554 (for example, iQ+, Butterfly Network, Guilford, CT) were used to visualize/capture images of the air bubble generation within a distilled, degassed water media.

In some embodiments, the transducer 400 provides rarefactional pressure level over 15 MPa for vaporization of general organ tissues. The histotripsy treatment was made within a relatively short time duration (<2 min), exhibiting the in-situ tumor removal within a precise treatment volume (<ϕ1×5 mm³). The histotripsy transducer 400 was configured to have strong ablation for histotripsy, based on the accumulated data from gene transfer tests and histotripsy tests with prototypes, expected to induce apoptotic death for all tumor cells in the treated area. Alternatively, a PMN-PT single crystal having a much higher piezoelectric constant (d33>2,500 pC/N) than that of PZT-based composite materials (d33<700 pC/N) was used to provide high negative pressure, and to consider a larger aperture (e.g., 8-10 Fr).

In another example method of treatment, the addition of systemic administration of ICB (anti-PD-1) to LM-HIFU+IT-IL-12 therapy enhances antitumor efficacy of ICB. In a previous study, anti-PD-1 antibody was combined with IT-IL-12 treatment, which not only significantly suppressed tumor growth in multiple TNBC tumor models, but also led to complete tumor regression and long-term tumor-free survival in some of the treated mice. Based on interactome analysis of TILs in the IT-IL-12 therapy, interactions between antigen presenting, myeloid populations and CD8 T cells were enriched, and CXCR3 gene signature was enhanced after IT-IL-12. Expression of CXCR3 gene signature was found in CD8 T cell rich tumors and was associated with improved PFS and OS in TNBC patients as well.

For ease of description, the transducers described in the example systems and methods below are referred to as the transducer 200 and the device 102A, 102B. It should be understood, however, that any suitable embodiment of the transducer 200 (for example, the transducers 300 and 400 described above) may be utilized in such systems and methods.

### Example 2—Methods of Use for Treatment of Cancer|_{[HJA(2]}

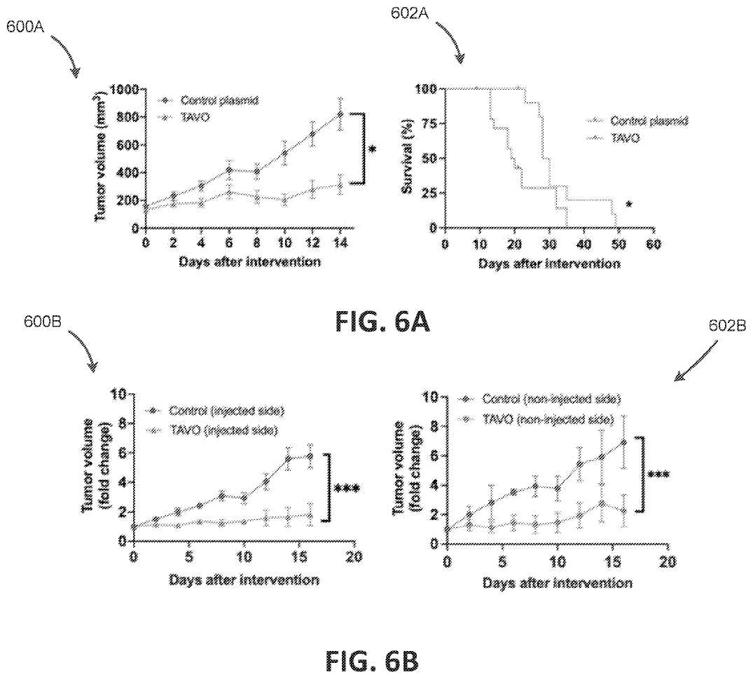

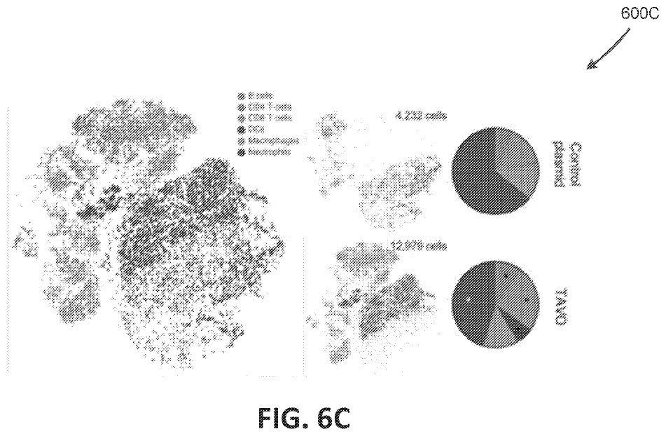

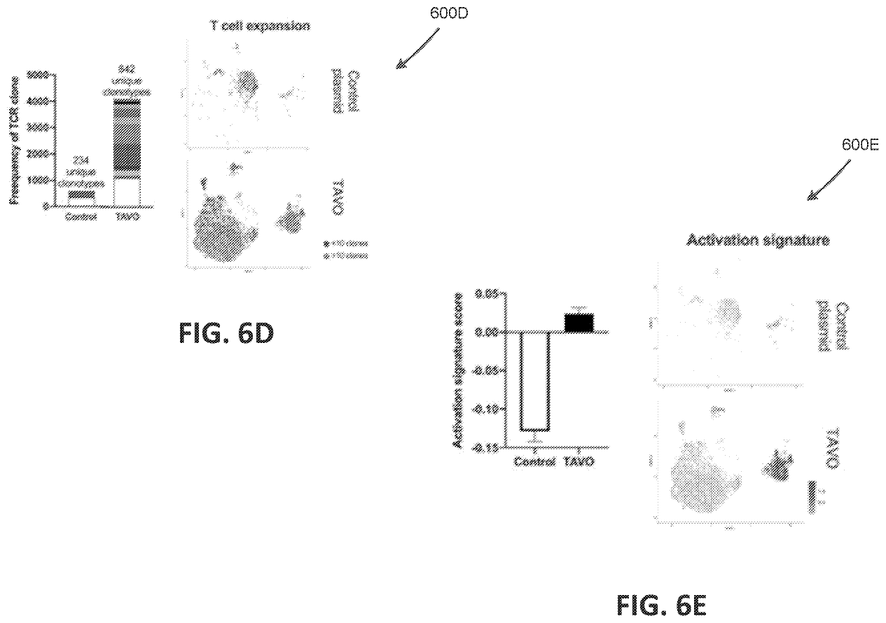

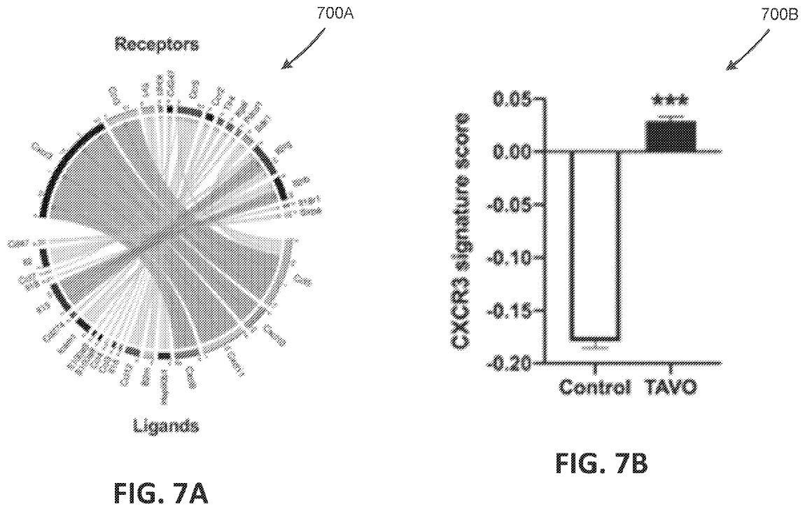

The example embodiments of HIFU devices including the transducer 200, 400 described hereinabove may be used in conjunction with immunotherapy for the multiple kinds of treatment including, for example, some types of cancers. In an example embodiment, a method of treatment comprises using a miniaturized transcatheter LM-HIFU devices (for example, the transducer 400 of FIGS. 4A and 5A) that can disrupt tumor tissues as well as induce efficient gene expression in tumor cells or tumor-infiltrating immune cells by efficient gene transduction. As described herein, it has been discovered that intratumoral administration of plasmid IL-12 with electroporation (IT-pIL12-EP, shown as TAVO in FIGS. 6A-6E, 7A-7B, and 8A-8D, which are described in more detail below) significantly suppressed the growth of triple negative BC (TNBC) in mice and prolonged survival (as illustrated in plots 600A and 602A of FIG. 6A). Plots 600A and 602A of FIG. 6A, in particular, depict data for Murine 4T1 tumor-bearing BALB/c mice that received intratumoral injections of mIL 12-P2A plasmid or control plasmid followed by in vivo electroporation on day 0 and growth inhibition of 4T1 tumors and survival of mice by the treatment (IL-12 group: 13 mice, control group: 14 mice). Moreover, the treatment provided protection in untreated lesions as well as treated lesion via induction of systemic antitumor immunity as shown in plots 600B (treated tumors) and 602B (untreated tumors) of FIG. 6B. Plots 600B and 602B, in particular, depict the abscopal effect of IT-pIL-12 administration. Tumor volumes were normalized to size at treatment initiation (on day 0) (n=4 for each group). The left graph depicts data for treated tumors whereas the right graph depicts data for tumors of the untreated side. Single-cell RNA-sequencing (scRNA-seq) on TILs revealed that IT-pIL 12-EP treatment induced a significant increase of T cells and dendritic cells, and decrease in neutrophils infiltration to the tumors, as shown in plots 600C of FIG. 6C (t-SNE plot of cells classified into cell types for all samples or divided by treatment group including proportional analysis of each cell type in each treatment group). T cells were more activated and clonally expanded in IT-pIL12-EP treated tumors compared to control treated tumors, supporting the hypothesis of adaptive immune response induced by local delivery of IL-12 (plots 600D and 600E of FIGS. 6D and 6E respectively). Plot 600D depicts (Lt) Quantification of the frequency of each clone with the top 50 clones for each treatment shown in colored bars. (Rt) UMAP showing reclustering of all TCR+ cells from colored by expansion of clones (<10-blue; >10-red). Plot 600E depicts (Lt) Quantification of activation signature score across all T cells in each treatment. (Rt) UMAP colored by 50 gene activation signature score. All error bars represent mean±SEM *p<0.05 ***p<0.001. More than 1600 genes were significantly upregulated following IT-pIL12-EP treatment, including genes associated with the increased CD8 T cell infiltration and activation, trafficking, antigen presentation, and exhaustion. KEGG pathway analysis further highlighted the enrichment of antigen presentation, cytokine, chemokine, and PDL1 pathways in IT-pIL12-EP treated tumors. Based on interactome analysis of scRNA-seq data, the top interactions between receptors on macrophages and ligands on CD8 T cells highlights the CXCL9/10/11/CXCR3 axis (plot 700A of FIG. 7A). Plot 7—A is a Circos plot depicting receptor-ligand interactions between receptors on CD8 T cells and ligands on macrophages. Connections shown in red represent the top 25% of interactions between these cell types. A CXCR3 gene signature and the top 50 genes with the highest correlation with CXCR3 were identified, and it was found that IT-pIL 12-EP treated tumors had a significantly higher CXCR3 signature score than cells from control treated tumors (as shown in plot 700B of FIG. 7B). Plot 700B illustrates 50 gene CXCR3 gene signature scores quantified across all cells. All error bars represent mean±SEM ***p<0.001.

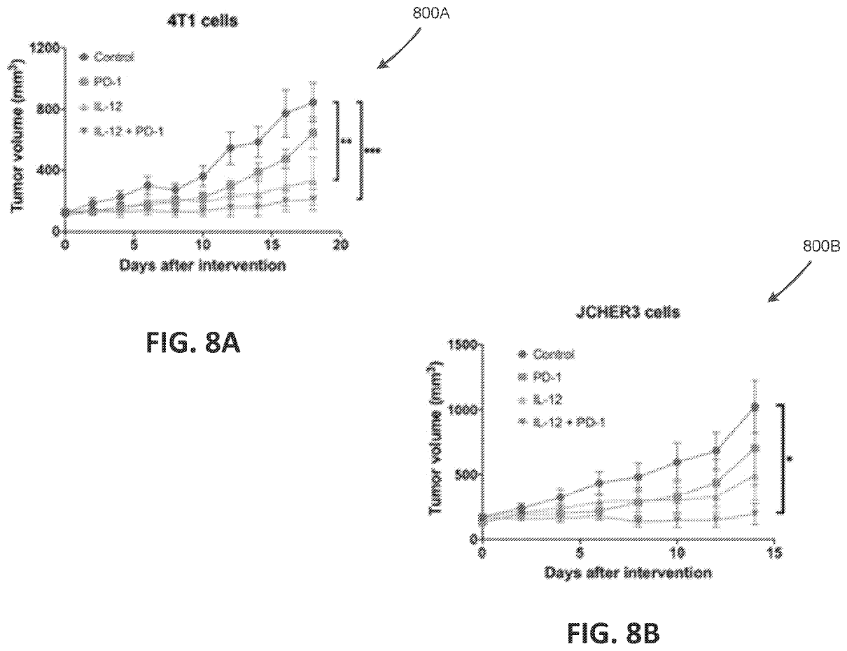

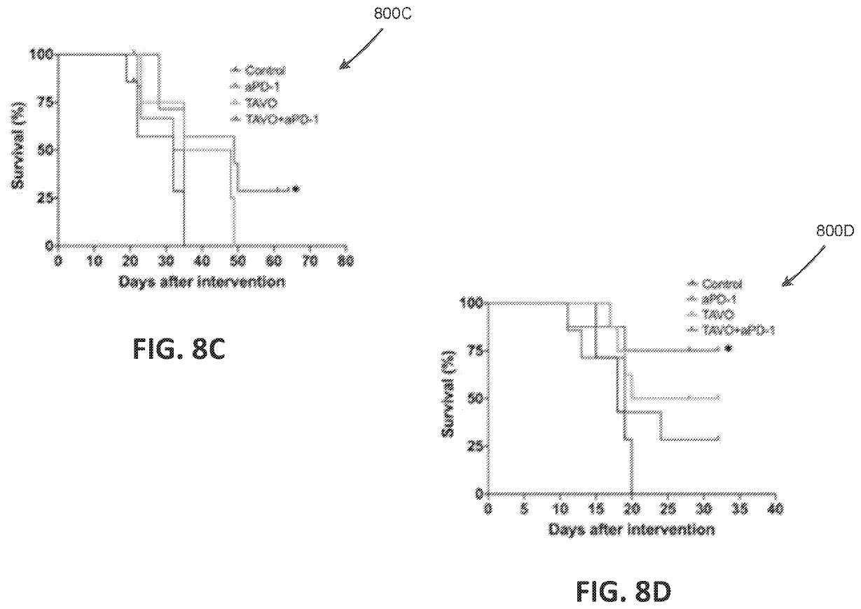

Overall, these analyses demonstrate an increase in the infiltration and activation of CD8 T cells by IT-pIL12-EP treatment, however the mixed expression of activation and exhaustion markers and enrichment of pathways like PD-1/PD-L1 indicate this therapy may be rationally combined with a checkpoint inhibitor to maximize therapeutic efficacy. To test this, tumor-bearing mice were treated with IT-pIL12-EP combined with anti-PD-1 antibodies. Plots 800A-800D of FIGS. 8A-8D respectively illustrate tumor curves from the combination therapy. Plot 800A depicts growth inhibition of 4T1 tumors by combination therapy. Tumor volumes were measured every other day (Control: 7 mice, anti-PD-1: 9 mice, IL-12:7 mice, Combination: 10 mice). Plot 800B depicts growth inhibition of JC-HER3 tumors by combination therapy. Tumor sizes were measured every other day (Control: 7 mice, anti-PD-1: 6 mice, IL-12:8 mice, Combination: 7 mice). Plots 800C and 800D depict mouse survival for each treatment arm is shown for 4T1 tumor model (C) and JC-HER3 tumor model (D). All error bars represent mean±SEM *P<0.05, **P<0.01, ***P<0.001. The combination not only significantly slowed tumor growth in multiple TNBC models, but it also led to complete tumor regression and long-term tumor-free survival in two different models of TNBC (28.6% in 4T1 model, 75.0% in JC-HER3 model).

The clinical safety of the therapy was tested in a single arm, prospective clinical trial of intratumoral pIL-12 (TAVO) monotherapy (OMS-140) in pre-treated locally advanced or recurrent TNBC. In addition to the demonstrated safety and feasibility of this pIL-12 monotherapy, an increase in CD8+ T cell infiltration and decrease in suppressive myeloid populations in tumor biopsies obtained from patients following pIL-12 monotherapy was observed in preclinical study.

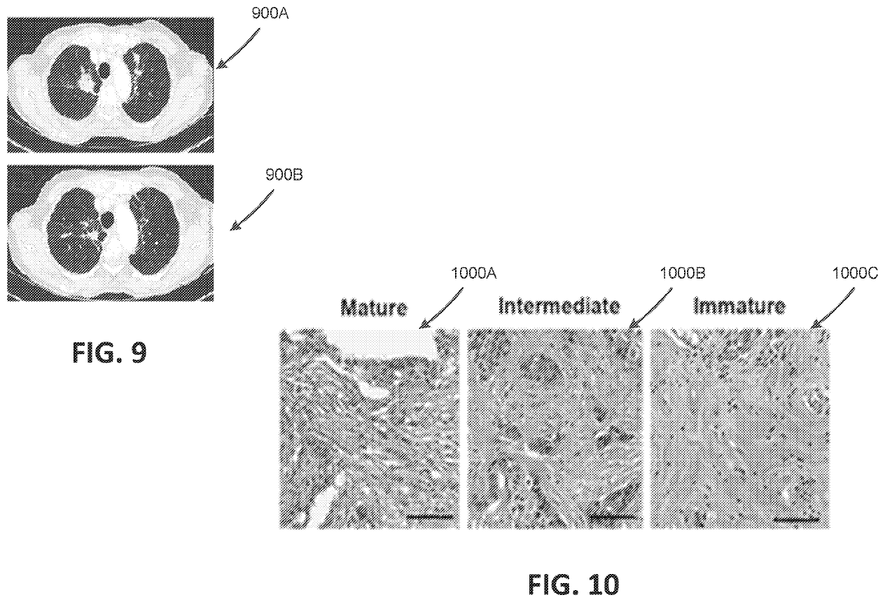

Systemic anti-tumor immune responses on distant, untreated tumors without any evidence of cytokine storm or other toxicity were also observed. Importantly, one patient who was previously unresponsive to anti-PD1 and exhibited increased CXCR3 gene signature scores by pIL-12 treatment, underwent anti-PD-L1 therapy as their immediate next treatment, and demonstrated a profound clinical response (as shown in images 900A and 900B of FIG. 9, wherein image 900A depicts an exemplary baseline image of lung metastases and image 900B depicts lung metastases after 1 cycle of IL-12 treatment and 3 months of nivolumab therapy). These data identify a gene signature that represents a functional biomarker to convert non-responders to responders.

### Example 3—Combining Disruption of the Tumor Microenvironment and Local Immunotherapy|_{[HJA(3]}

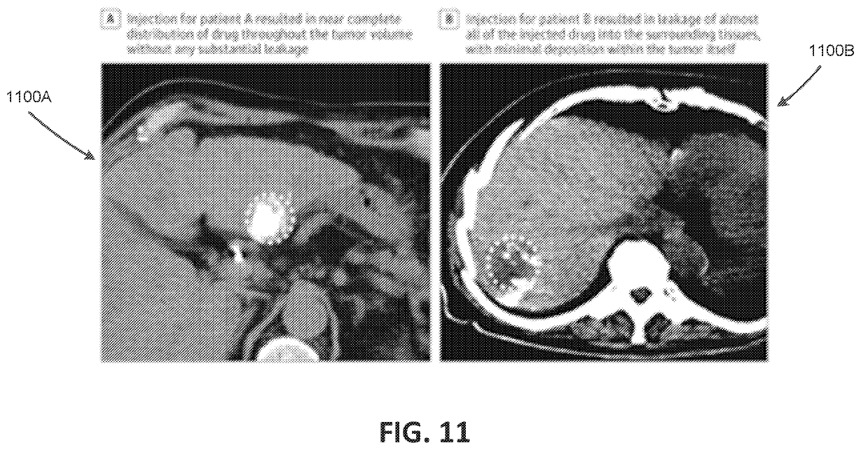

One barrier to the use of therapeutics is the tumor microenvironment (TME), which is comprised of cancer cells as well as a stromal compartment which includes fibroblasts, myofibroblasts, leukocytes, endothelial cells, macrophages, adipocytes and extracellular matrix (ECM). Breast cancers have various levels of stroma, as shown in FIG. 10, which includes images 1000A-1000C, which each depict fibrotic tumor stromal in breast cancer at varying stages), and ECM formed by stromal proteins (collagen, elastin, fibronectin and laminin), which creates a physical barrier that prevents intratumoral drug distribution and direct contact between tumor cells and drug or tumor-infiltrating immune effector cells. As shown in images 1100A and 1100B of FIG. 11, intratumoral injections of drug often failed with leakage of drug to surrounding tissues and very little drug delivered into the tumors. Image 1100A depicts near-complete filing while image 1100B depicts minimal drug retention with leakage.

It has been demonstrated that noninvasive HIFU directed at solid tumors as well as IT-IL-12 gene delivery could be used to enhance immune infiltration and elicit a systemic anti-tumor immune response. HIFU could also aid in the destruction of the stromal barrier in tumors, and could enhance IT-IL-12 gene therapy through improved distribution of IL-12 expression vectors in tumors.

With the intention of disrupting the stromal barrier as well as tumor cells and enhancing immune infiltration, a novel HIFU strategy which mechanically disrupts cell and stroma, in contrast to conventional thermal HIFU (T-HIFU) therapy that results in coagulation necrosis, was determined and is herein described.

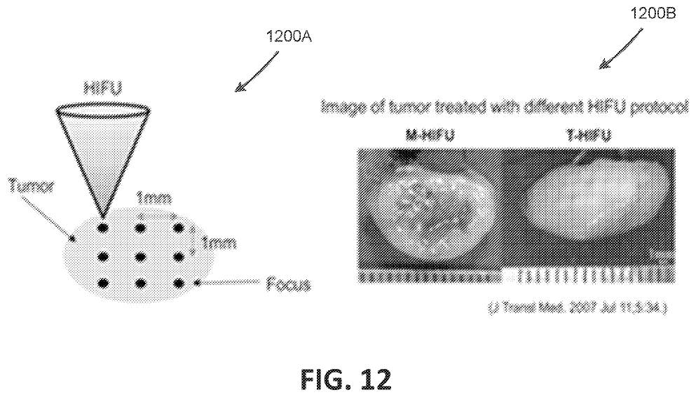

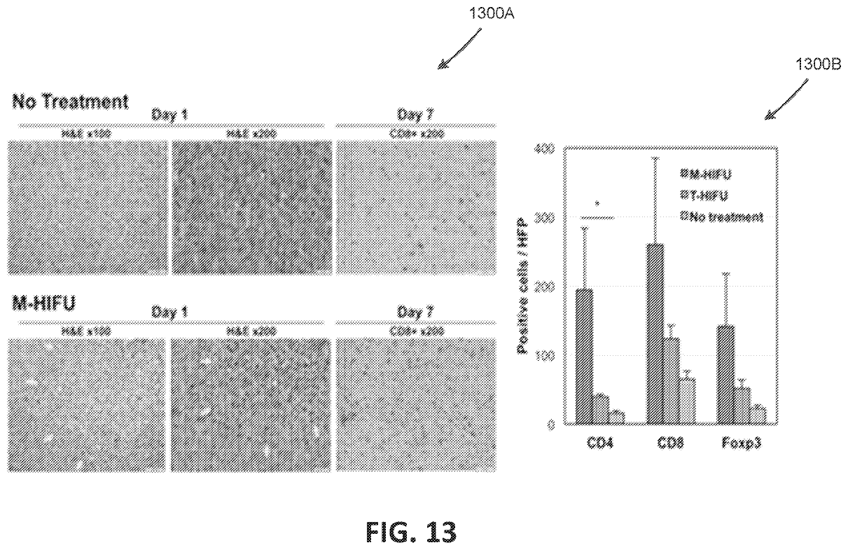

An extracorporeal HIFU system was used (VIFU2000 wet system for small animals, Alpinion) and mechanical disruption was achieved through acoustic cavitation of the TME, enabled by the use of high-pressure burst exposure which has been termed mechanical HIFU, M-HIFU or histotripsy. M-HIFU induces mechanical damage to the tumor tissues and tumor stroma and vessels, resulting in cavitation with internal bleeding in an immune competent model of BC (FIG. 12, which includes images 1200A and 1200B illustrating how Murine MC38 tumors grown on a thigh were treated by M-HIFU and T-HIFU). Histological analysis of tumor tissues harvested 1, 7 or 11 days after M-HIFU treatment revealed enhanced immune infiltration, including T cell infiltration into tumors (as shown in graph 1300A and table 1300B of FIG. 13). As illustrated in the graph 1300A, H & E staining of tumor tissues a day after M-HIFU treatment. The arrows indicate the mechanically destructed area, which has infiltrate of immune cells. Increase of CD8+ T cell infiltration was evident on day 7 after M-HIFU. As depicted in table 1300B, averages of CD4, CD8, or FoxP3 staining+ cells (mean of 5 HPFs for each tumor) for each treatment are shown. Bar represents mean±SE, n=3. *: p value<0.05.

MM3MG-HER2 cells were implanted to right thigh and left flank of female BALB/c mice. 7 days later, M-HIFU was performed to right thigh tumors in M-HIFU monotherapy or combination group. Anti-PD-L1 antibody (100 μg/inj) or Isotype control IgG (100 μg/inj) was injected ip on day 10, 13 and 16. Tumor growth curves of HIFU-treated thigh tumors and untreated distant tumors are shown. The data in graphs 1400A and 1400B represents results from two pooled experiments; n=13 to 19 per group. Error bar: mean±SD. *: P<0.05, **: P<0.01, ***: P<0.001, *****P<0.0001.

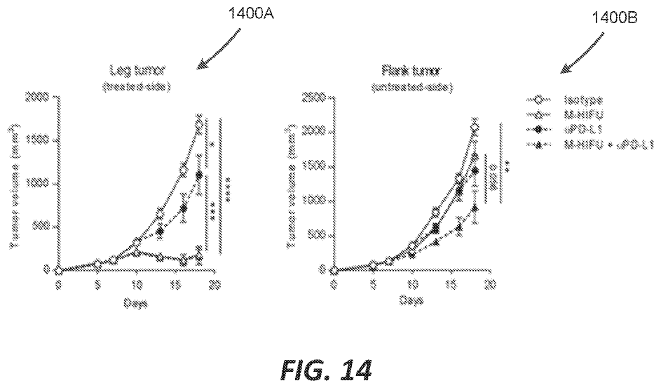

The superior antitumor efficacies of tumor growth suppression and improved survival of M-HIFU compared to conventional T-HIFU was demonstrated in the immune competent MM3MG-HER2 murine breast cancer model. A modest abscopal effect on untreated distant tumors by M-HIFU monotherapy was observed, which could be augmented by the combination treatment of M-HIFU and PD-1/PD-L1 blockade, as shown in plots 1400A and 1400B of FIG. 14.

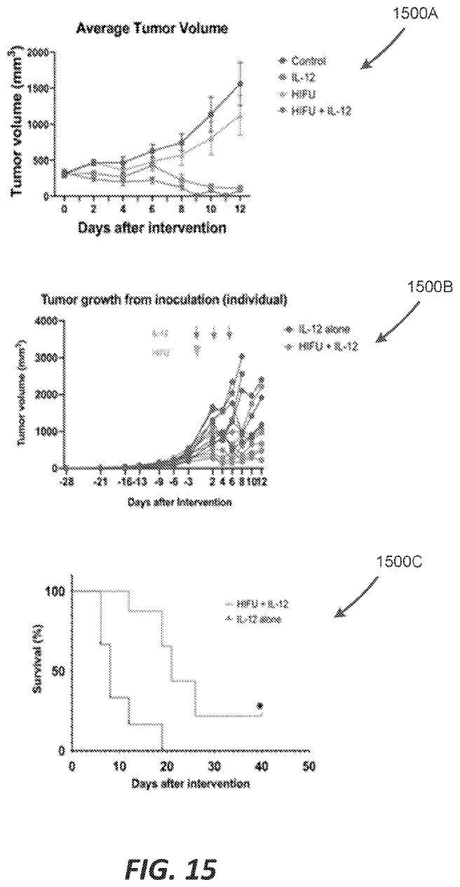

As both IT-IL-12 gene therapy and M-HIFU treatment can induce antitumor efficacy through non-overlapping mechanisms, and both modalities appeared to synergize with anti-PD-L1 antibody therapy, it was hypothesized that combining M-HIFU with IT-IL-12 gene therapy would enable wider distribution of IL-12 expression vectors into tumors and induce more intratumoral T cell infiltration by the disruption of stromal barrier. To test this hypothesis, E0771 tumor-bearing mice were treated with IL-12 gene therapy alone, M-HIFU alone, or a combination of both. As shown in plot 1500A of FIG. 15, the strongest tumor growth suppression in combination treatment group, although no statistical difference was confirmed between IL-12 alone and combination treatment with this relatively early start of intervention. Next, a relatively late intervention to E0771 tumor-bearing mice (28 days) was made (plots 1500B and 1500C) when tumors were around 500 mm³, and found that the combination of IL-12 gene therapy and M-HIFU induced stronger tumor growth suppression and more prolonged survival of mice compared to IL-12 gene therapy alone.

With reference to plot 1500A, E0771 tumor-bearing C57BL/6 mice were treated with IT-IL-12 gene therapy alone, M-HIFU alone or combination of them 28 days after tumor cell implantation. Tumor volume was monitored every two days. n=4-5 mice. Combination treatment suppressed tumor growth significantly better than HIFU alone (p<0.01). With reference to plot 1500B, intervention was initiated 28 days after E0771 cell implantation. Mice received either IL-12 gene therapy alone or combination of IL-12 and HIFU. N=8 mice. Combination treatment induced significantly stronger tumor growth suppression (AUC analysis, t-test: p<0.0001). Finally, with reference to plot 1500C, mouse survival is shown. Log-rank test: p<0.005.

As shown in images 1600A and 1600B of FIGS. 16A and 16B respectively, an example treatment strategy against, for example, metastatic breast cancers (BC) is illustrated. The disclosed method comprises: a transcatheter HIFU transducer (for example, the transducer 200, 400) integrated with a catheter (for example, device 102 of FIGS. 1A and 1B) which is inserted into the metastatic tumors (such as liver, lung, brain or bone metastasis) under ultrasound imaging or MR imaging 1602, histotripsy (M-HIFU) was conducted to destroy tumor cells and tumor stromal barrier, and IL-12 expression vectors are locally delivered to induce enhanced expression of IL-12 in the TME. The miniaturized HIFU transducer 200 can be directly inserted to the primary or metastatic BC even if they are deep-seated or located in difficult sites to target by extracorporeal HIFU, such as hepatic dome, posterior to ribs, near large blood vessels, or deep in lungs.

As illustrated in images 1600A and 1600B in particular, a transcatheter HIFU transducer was inserted to the tumor site under ultrasound imaging or MR imaging with a guiding needle. Histotripsy (M-HIFU) was conducted to destroy tumor cells and tumor stromal barrier, and IL-12 expression vectors were infused into tumors through another lumen of the catheter Tumor destruction and induced inflammatory TME and recruited cytotoxic immune cells eradicated the treated tumors. Induced systemic antitumor immunity which was enhanced by IT-IL-12 was expected to eradicate distant metastatic tumors.

To enhance the systemic antitumor immunity induced by IT-IL-12, extracorporeal M-HIFU was combined with IT-IL-12 and found that the combination treatment was more potent than IT-IL-12 alone. Thus, this combination strategy was proposed to treat metastatic BC. This local therapy can be applied in metastatic breast tumors located in or on visceral organs, such as liver, lung and brain. A transcatheter HIFU device, such as those described herein (for example, a device 102 including the transducer 200, 400), provides localized delivery of M-HIFU as well as IL-12 gene delivery into tumors.

### Example 4—In Vivo Functionality of Miniaturized HIFU Transducer|_{[HJA(4]}

The TME altered by LM-HIFU+IT-IL-12 therapy alone and in combination with ICB was analyzed using a combination of pathological analyses and scRNA sequencing to confirm in vivo functionality of the catheter LM-HIFU device 102. Next, antitumor efficacy and induced tumor-specific immune response by LM-HIFU+IT-IL-12 combination therapy with/without anti-PD-1 mAb was assessed and demonstrated abscopal effect and inhibition of pulmonary metastasis in TNBC model. Finally, by pathological analysis and scRNA-seq analysis, it was assessed whether significant modifications of TME in LM-HIFU+IT-IL-12 treated tumors, and increased CXCR3 gene signatures in TILs would be found.

E0771 BC cells were subcutaneously implanted to the flank of female C57BL/6 mice. Once tumors reached 7-8 mm in size, tumors were treated with LM-HIFU using the transcatheter device 102 with simultaneous infusion of Ad-IL-12 (1010 viral particles in 50 μL saline/tumor) into tumors through a dual-lumen catheter. The following groups were made; a) No treatment, b) LM-HIFU alone, c) IT-IL-12 alone, and d) LM-HIFU+IT-IL-12. Mice in Group b had only LM-HIFU without Ad-IL-12 infusion, and Group c had only Ad-IL-12 infusion without HIFU energy. (n=8 mice for each group, 4 mice for 3-day and 4 mice for 7-day termination). On days 3 and 7 after the procedure, 4 mice of each group were euthanized on each day, and tumors and blood were collected for the assays.

A half of tumor tissues were used for pathological analysis to evaluate tumor tissue destruction and T cell infiltration into the tumors by IHC, as well as to assess IL-12 expression by in situ hybridization (RNAscope). A quarter of tumor tissue was enzymatically digested and isolated immune cells be analyzed by flow cytometry. Using tumor lysate and blood serum, the level of IL-12 protein was analyzed by IL-12 ELISA. It was confirmed whether disruption of tumor tissue and enhanced T cell infiltration into tumors by LM-HIFU treatment at the earlier time point and increased IL-12 levels in IT-IL-12-treated tumors but not in blood. How tumor-infiltrating immune cells are modified by LM-HIFU+IT-IL-12 using single cell secretome was determined.

The induced systemic tumor-specific immune response and antitumor efficacy of combined LM-HIFU+IT-IL-12 therapy against murine TNBC was demonstrated by the following method. E0771-OVA murine BC cell line was implanted to the flank of female mice (C57BL/6). When tumor sizes of the right flank reach approximately 7-8 mm in diameter, LM-HIFU or IT-IL-12 treatment was carried out as monotherapy or combination therapy to the tumors. A catheter with a miniaturized HIFU transducer and a drug infusion lumen was inserted to tumors, and LM-HIFU and/or IT-IL-12 was conducted.

The following groups were tested; a) No treatment, b) LM-HIFU alone, c) IT-IL-12 alone, and d) LM-HIFU+IT-IL-12 (n=14 mice for each group, 4 mice among them for 7-day termination). Four mice from each arm were euthanized 7 days after the treatment. Tumors, spleen and blood were collected, and IFN-γ ELISPOT assays (OVA peptide (SIINFEKL) as stimulating antigens), ELISA (antibody for OVA protein) and flow cytometry with H-2Kb/OVA (SIINFEKL) MHC tetramer were performed. Tumor samples were analyzed for the IL-12 production by in situ hybridization (RNAscope) and IL-12 ELISA using tumor lysates as samples. The number of tumor-infiltrating leukocytes analyzed by flow cytometry and immunohistochemistry (THC). After digesting tumor tissues with triple-enzyme buffer (collagenase III, hyaluronidase, DNase), multi-parameter flow cytometry will be performed on cells isolated from tissues for CD4 T-helper cells (Th1: CD4/CCR5, Th2: CD4/CCR4), CD8 CTLs (central memory: CD8/CD62L, effector memory: CD8/KLRG1), dendritic cells (CD11c/CD80/CD86), B cells (CD19), and NK cells (CD49b/NKG2D), regulatory T (Treg) cell (CD4/CD25/FoxP3), tumor associated macrophages (TAMs) (CD11b/F4-80/CSF-1R), myeloid-derived suppressor cells (MDSCs) (CD11b/Gr-1) to characterize immune cell population. Activation status of T cells will be analyzed with anti-CD25, CD69, ICOS, PD-1, CD107, and Granzyme-B. Expression of immune checkpoint molecules, CTLA-4, PD-L1, TIM3, LAG3, and TIGIT were also analyzed as markers of cell exhaustion. Expression of immune checkpoint molecules, CTLA-4, PD-L1, TIM3, LAG3, and TIGIT will also be analyzed as markers of cell exhaustion. Cells from spleens were also analyzed via flow cytometry and compared with TILs. In addition, fluorescence microscope analysis were performed by staining tumor tissues using fluorescence-labeled anti-CD3, CD4, CD8, CD11b, CD11c, CD19, CD49b, F4/80, Ly6G, FoxP3 antibodies and antibodies for immune checkpoint molecules. The interaction of different immune cell types as well as the total numbers of immune cells in TME were assessed. Photographs were taken and the number of stained cells were analyzed using Image J software. Multiple cell lineages (CD4+T, CD8+T, Treg, NK, DC, macrophage, MDSC) were identified and quantified.

For scRNA-seq, tumor tissues were digested as described above, and alive tumor infiltrating immune cells (viability dye-, CD45+ cells) were FACS sorted. 10× libraries were created using Chromium Single Cell 5′ Library Construction Kit (v1.1) following manufacturer's protocol. Both gene expression and V(D)J enrichment libraries were created for each sample. Generated cDNA and final GEX/TCR libraries were quality checked using an Agilent Bioanalyzer 2100 and were sequenced on a NovaSeq S4 instrument. Fastq files from 10× library sequencing were processed using Partek® Genomics Suite® software (version 9.0.20, Copyright©; 2018 Partek Inc). Identified clusters were visualized using UMAP plots using first 15 principal components, a minimum distance of 0.4, 30 neighbors 42 Differentially expressed genes were identified using the GSA algorithm in Partek Flow. KEGG pathway enrichment analysis was performed on genes found differentially expressed at FDR 5% with a fold change >|2|. Clustering, cell type identification, and differential gene expression analysis of single cell data were done with treatment groups deidentified for blinding. Samples from four groups were compared. Because increased CXCR3 gene signature with enhanced antigen presentation was identified, expansion and licensing of T cells systemically in IT-IL-12 treated TNBC in a previous study, it was assessed if combined LM-HIFU+IT-IL-12 treatment using the catheter LM-HIFU device would induce similar modifications in TME.

For the other 10 mice, tumor sizes were measured three times a week until they reached humane endpoint (tumor volume>2,000 mm³) and survival of mice were assessed. Percent tumor growth inhibition (% TGI) which is defined as the percent difference between the median tumor volumes (MTVs) of treated and control mice were also compared. To confirm the induction of potent systemic antitumor immunity, long-term survivors after tumor eradication will receive tumor rechallenge 12 weeks after the LM-HIFU+IT-IL-12 treatment. Tumor growth/tumor-free survival were monitored for up to two months. The task was intended to confirm the superiority of combined LM-HIFU+IT-IL-12 treatment over IT-IL-12 monotherapy or LM-HIFU monotherapy for the induction of antitumor immunity and tumor growth suppression.

The abscopal effect induced by LM-HIFU+IT-IL-12 therapy and enhanced antitumor efficacy by combination with anti-PD-1 antibody in bilateral TNBC implantation model was demonstrated as follows. E0771-OVA cells were bilaterally implanted to the flank of female C57BL/6 mice. When tumor sizes of the right flank reach approximately 7-8 mm in diameter, LM-HIFU+IT-IL-12 treatment with or without anti-PD-1 antibody were carried out to the tumors in the right flank, but contralateral tumors were left untreated.

The following groups were tested; a) no treatment, b) anti-PD-1 mAb alone, c) LM-HIFU+IT-IL-12+control IgG, and d) LM-HIFU+IT-IL-12+anti-PD-1 mAb (n=14 mice for each group, four mice among them for 7-day termination). Anti-PD-1 Ab or control IgG (200 μg/inj) were intraperitoneally administered on day 3 after LM-HIFU+IT-IL-12 treatment and repeated twice a week for two weeks.

As described hereinabove, four mice from each group were euthanized seven days after the treatment, and collected tumor samples from both treated local sites and untreated remote sites were analyzed. Collected splenocytes and blood serum were used for IFN-γ ELISPOT assays, tetramer assay and ELISA. scRNA-seq analysis and multi-parameter flow cytometry were performed for TILs. TILs isolated from treated tumors and untreated distant tumors were analyzed and compared for four treatment groups (only Group A will have 1 TIL sample). It was assessed if LM-HIFU+IT-IL-12 therapy combined with anti-PD-1 would show more significant modifications of TME compared to LM-HIFU+IT-IL-12 therapy alone. Differences in the modification of TME between treated tumors and untreated distant tumors were also analyzed.

For the other 10 mice, tumor sizes (both HIFU treated local tumors and untreated distant tumors) were monitored three times a week until they reached humane endpoint (tumor volume>2,000 mm³), and mouse survival were compared between the arms. % TGI were also compared. To confirm the induction of potent systemic antitumor immunity, long-term survivors after tumor eradication received tumor rechallenge 12 weeks after the LM-HIFU+IT-IL-12 treatment. Tumor growth/tumor-free survival were monitored for up to two months.

This task was intended to confirm the superiority of combining anti-PD-1 antibody with LM-HIFU+IT-IL-12 therapy over LM-HIFU+IT-IL-12 therapy or anti-PD-1 antibody alone for the induction of systemic antitumor immunity and growth inhibition of distant tumors (abscopal effect).

Inhibition of spontaneous pulmonary metastasis by LM-HIFU+IT-IL-12 therapy with anti-PD-1 antibody in highly metastatic E0771-LX3-OVA model was demonstrated as follows. After three rounds of orthotopic implantation of cells isolated from pulmonary metastasis of E0771 cells in C57BL/6 mice, highly metastatic variant, named E0771-CH3-MX3, was established in the lab. It was confirmed this metastatic variant makes pulmonary metastasis by 5-6 weeks after orthotopic implantation (5×10E5 cells/inj) for all the mice tested. E0771-CH3-MX3 cells were implanted to 4th mammary fat pad of female C57BL/6 mice. When tumors grew approximately 7-8 mm in diameter, LM-HIFU+IT-IL-12 treatment with or without anti-PD-1 antibody were carried out. The following groups were tested; a) no treatment, b) anti-PD-1 mAb alone, c) LM-HIFU+IT-IL-12+control IgG, and d) LM-HIFU+IT-IL-12+anti-PD-1 mAb (n=10 mice for each group). Anti-PD-1 Ab or control IgG (200 μg/inj) were intraperitoneally administered on day three after LM-HIFU+IT-IL-12 treatment and repeated twice a week for two weeks. Tumor growth as well as mouse activity, respiratory rate, appetite and mouse body condition were monitored. Mice were euthanized six weeks after tumor cell implantation, and collected lungs were macroscopically and microscopically evaluated for pulmonary metastasis Tissue sections were analyzed by H&E staining and IHC with anti-p53 antibody (E0771 cell line: p53+), and metastatic foci were quantified using Image J software. Fluorescence microscope analysis was performed by staining frozen tumor tissues with fluorescence-labeled anti-CD3, CD4, CD8, CD49b, and granzyme B antibodies to determine the effector immune cells in metastatic nodules. Photographs were taken and the number of stained cells were analyzed using Image J software.

An optimized intralesional immunotherapy was used to investigate antitumor efficacy of LM-HIFU+IT-IL-12 treatment against ICB-less responsive ER+BC and HER2+ BC and also confirm the induction of systemic antitumor immunity and abscopal effect by this combination treatment.

Ovalbumin-expressing murine triple-negative BC cell line E0771 (E0771-OVA) was established using lentiviral vector as described before. Utilizing a non-transformed BALB/c breast epithelial line (MM3MG), HER2-driven BC cell line, MM3MG-HER2 was established by retroviral transduction of receptor tyrosine-protein kinase erbB-2 (HER2). OVA and HER2 antigens were used in assays to evaluate the induction of systemic antitumor immunity.

Using DNA deep-sequencing technology, recent studies have identified several conserved mutations to the DNA-binding domain of ESR1 in a significant proportion (˜30-40%) of endocrine-resistant metastatic ER+ breast cancer patients. As no established models of endocrine-resistant ER+ breast cancer existed in immunocompetent mice, an ER+ endocrine resistant breast cancer cell line model was generated that would grow in immune competent syngeneic animals. MM3MG cells were artificially transformed with wild-type or mutated human ESR1 (Y537N, Y537S, D538G).

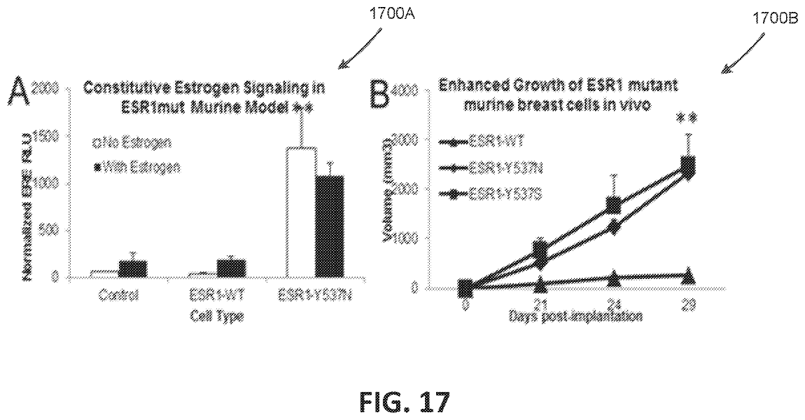

As depicted in plot 1700A of FIG. 17, lentiviral stably transduced pre-malignant MM3MG cells (BALB/c background) were transfected with ERE Luciferase reporters along with transfection controls in estrogen free condition or positive (20 nM E2) conditions and harvested at 24 h (N=6). In plot 1700B of FIG. 17, these cells were implanted in female mice (no estrogen pellets) and tumor growth assessed over time by caliper measurement. In all panels * represents p<0.05 and **p<0.01 from ESR1-WT controls.

In this model, it was found that these ESR1 mutants conferred enhanced estrogen signaling in these cell lines (plot 1700A of FIG. 17) and in fact, ‘transformed’ them, permitting them to form more aggressive tumors that successfully engrafted into mice when compared to their control and Wild-Type ESR1 counterparts (plot 1700B of FIG. 17). Critically, these mice did not require supplementation of exogenous estrogen to permit estrogen-mediated signaling or tumor growth. It was also found that these cells were completely resistant to standard endocrine therapies (e.g., Tamoxifen and aromatase inhibitors (data not shown)). Therefore, the established murine breast cancer cell lines, MM3MG-ESR1mut (Y537N, Y537S, D538G), simulate patients' metastatic ER+ breast cancers, that are endocrine-resistant, having ESR1 gene mutation, and behave more aggressively.

The induced systemic tumor-specific immune response and antitumor efficacy of combined LM-HIFU+IT-IL-12 therapy against ER+ and HER2+ murine breast cancers were demonstrated as follows. Murine BC cell lines, MM3MG-ESR1mut (Exp 3.1) and MM3MG-HER2 (Exp 3.2), were implanted to the right flank of female BALB/c mice. LM-HIFU or IT-IL-12 treatment was carried out as monotherapy or combination therapy to the tumors as described hereinabove for TNBC. A catheter LM-HIFU device with a drug infusion lumen was inserted to tumors, and combined LM-HIFU+IT-IL-12 treatment was conducted in the optimized setting.

The following groups were tested; a) No treatment, b) LM-HIFU alone, c) IT-IL-12 alone, and d) LM-HIFU+IT-IL-12 (n=14 mice for each group, four mice among them for 7-day termination). Four mice from each arm were euthanized 7 days after the treatment. Tumors, spleen and blood were collected, and IFN-γ ELISPOT assays (ESR1 peptide mix or HER2 peptide mix as stimulating antigens for Exp 3.1, 3.2, respectively) and Cell-based ELISAs (with parental E0771/E0771-ESR1 cells or parental 4T1/4T1-HER2 cells for Exp 3.1 and 3.2, respectively, both cell lines available) were performed. Tumor samples were analyzed for the IL-12 production by in situ hybridization (RNAscope) and IL-12 ELISA using tumor lysates as samples. To analyze cell types and activation/maturation status of TILs, flow cytometry analysis was performed as described hereinabove. Tumor-infiltrating leukocytes were further analyzed by fluorescence microscopy. scRNA-seq analysis was also be performed. By pathological analysis, flow cytometry and scRNA-seq analyses, it was determined if ER+ and HER2+ tumors, compared to TNBC, responded to LM-HIFU+IT-IL-12 treatment in a similar way, such as increased intratumoral T cell infiltration, modifications of TME, and induction of antigen-specific immune response.

For the other 10 mice, tumor sizes were measured three times a week until they reached humane endpoint (tumor volume>2,000 mm³) and survival of mice were assessed. Percent tumor growth inhibition (% TGI) was compared. To confirm the induction of potent systemic antitumor immunity, long-term survivors after tumor eradication received tumor rechallenge 12 weeks after the LM-HIFU+IT-IL-12 treatment. Tumor growth/tumor-free survival was monitored for up to 2 months.

The abscopal effect induced by LM-HIFU+IT-IL-12 therapy and enhanced antitumor efficacy by combination with anti-PD-1 antibody in bilateral ER+ and HER2+ models can be demonstrated as follows. MM3MG-ESR1mut or MM3MG-HER2 cells were bilaterally implanted to the flank of female BALB/c mice (Exps 3.3, 3.4). As described hereinabove, LM-HIFU+IT-IL-12 treatment with or without anti-PD-1 antibody was carried out to the tumors in the right flank, but contralateral tumors were left untreated.

The following groups were tested; a) no treatment, b) anti-PD-1 mAb alone, c) LM-HIFU+IT-IL-12+control IgG, and d) LM-HIFU+IT-IL-12+anti-PD-1 mAb (n=14 mice for each group). Anti-PD-1 Ab or control IgG (200 μg/inj) was intraperitoneally administered on day three after LM-HIFU+IT-IL-12 treatment and repeated twice a week for two weeks.

Four mice from each group were euthanized 7 days after the treatment, and collected tumor samples from both treated local sites and untreated remote sites were analyzed. Collected splenocytes and blood serum were used for IFN-γ ELISPOT assays and ELISA. scRNA-seq analysis were performed for TILs isolated from both treated tumors and untreated distant tumors of each group. It was assessed if LM-HIFU+IT-IL-12 therapy combined with anti-PD-1 would show more significant modifications of TME compared to LM-HIFU+IT-IL-12 therapy alone. CXCR3 gene signature level was also assessed. Differences in the modification of TME between treated tumors and untreated distant tumors in each group was also assessed.

For the other 10 mice, tumor sizes (both HIFU treated local tumors and untreated distant tumors) were measured three times a week until they reached humane endpoint (tumor volume>2,000 mm³), and mouse survival were compared between the arms. % TGI were also compared. To confirm the induction of potent systemic antitumor immunity, long-term survivors after tumor eradication received tumor rechallenge. Tumor growth/tumor-free survival were monitored for up to two months. In this task, it was intended to confirm that the combination of anti-PD-1 antibody with LM-HIFU+IT-IL-12 therapy would induce the strongest antitumor effect, systemic antitumor immunity and abscopal effect, in ER+ and HER2+BC models. It was also investigated if ER+ and HER2+BC would show different modifications of TME compared to TNBC after the combination treatment.

### Example 5—Intracorporeal Sonoporation-Induced Drug/Gene Delivery Using Miniaturized HIFU Transducer|_{[HJA(5]}

As noted above, the typical US transducer for sonoporation (e.g., single-element focused US transducer) is extracorporeal, which is not suitable for intratumoral immunotherapy. Several limitations of the extracorporeal US transducer include that it cannot sonicate the tumors behind bones and fat efficiently due to ultrasound attenuation and absorption and it cannot precisely inject MBs and nucleic acids into the US treatment zone at the same time, which may miss the target. These issues lower the effectiveness of drug/gene delivery and increase undesired systemic toxicity for cancer immunotherapy.

Thus, the systems and methods described herein are directed to a miniaturized US transducer (for example, the transducer 400 described above) integrated into a catheter (i.e., catheter US transducer) for intracorporeal sonoporation-induced drug/gene delivery in intratumoral immunotherapy.

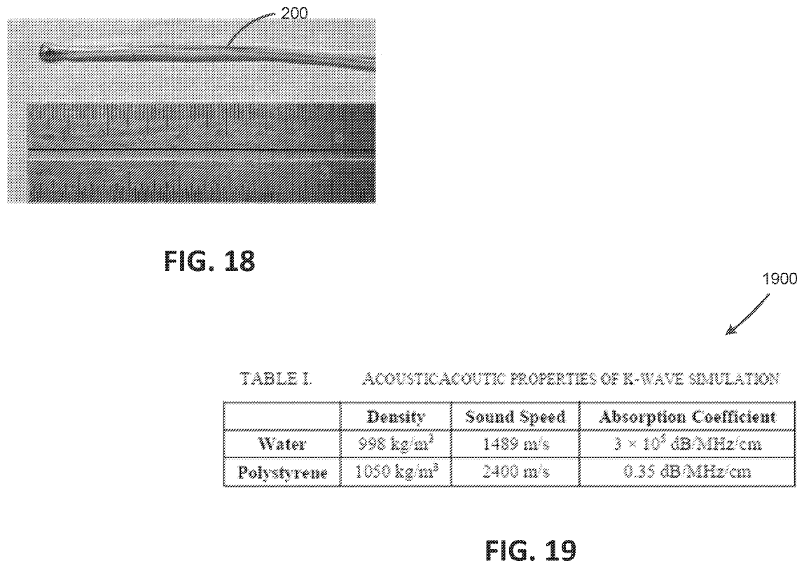

An 800 KHz US transducer with an aperture size of 2×2 mm² (similar to the transducer 400 of FIG. 5E) was designed and fabricated for intracorporeal sonoporation studies. The transducer 400 in the embodiment illustrated in FIG. 18 includes double layered PZT-5A, matching (Al₂O₃/epoxy), and backing (air bubble/epoxy) layers (not shown) as described above in regard to FIGS. 4A and SA. To inject microbubbles into the sonication area, a lumen was embedded inside the catheter, next to the US transducer. The lumen size was about 0.9 mm (outer diameter), while the catheter size was about 3.0 mm (outer diameter). The output acoustic pressure of the developed transducer was characterized by a calibrated hydrophone (HNA-0400, ONDA Corporation, CA, USA) in degassed water.

Since US standing waves may impact in-vitro sonoporation efficiency, an acoustic simulation was first conducted using the k-Wave toolbox to explore the 800 kHz US wave propagation in a 384-well cell culture plate. Given the axial symmetry boundary condition of each well in the cell culture plate, the computational domain was set as a 3.6 mm×7.6 mm 2D plane filled with water. The material of the culture plate was polystyrene and its acoustic properties including density, sound speed, and absorption coefficient were summarized in table 1900 of FIG. 19. The US transducer was placed on the top of the well, with US frequency of 800 kHz and input pressure of 0.4 MPa.

Human embryonic kidney (HEK) 293T cells and plasmid DNA, encoding green fluorescent protein-luciferase (GFPLUC) were prepared. HEK 293T cells (12,000 cells/well, 3×105 cells/mL, 40 μL/well) and plasmid DNA (0.4 μg/well, 20 μg/mL, 20 μL/well) were plated in a 384-well plate for sonoporation tests. VesselVue® MBs (SonoVol, Inc., NC, USA) with a mean diameter of 1.01±0.59 μm were used as cavitation nuclei in this study. MBs were mixed with phosphate-buffered saline (PBS) solution in a 10 mL syringe for tube injection, and the MBs concentration was diluted to 8.73×108 bubbles/mL. During sonoporation tests, 30 μL MBs solution was infused into each well through the injection tube, and the total solution volume in each well was approximately 90 μL.

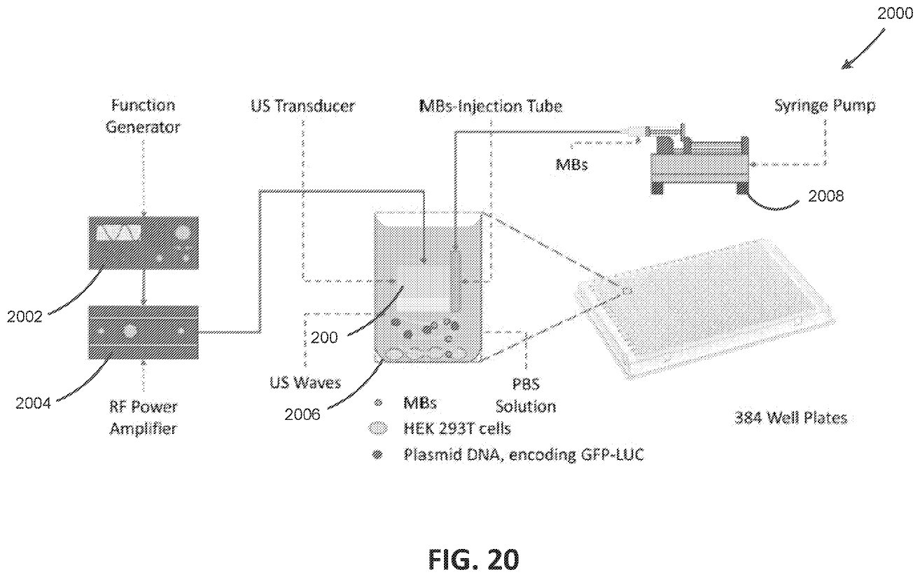

As it is shown in the test system 2000 of FIG. 20, pulsed electrical waveforms first generated by a function generator 2002 (e.g., 33250A, Agilent Technologies, Inc., CA, USA), and then amplified by an RF power amplifier 2004 (e.g., 75A250A, Amplifier Research Corporation, PA, USA), were utilized to drive the catheter US transducer 200. The catheter US transducer 200 was inserted into the cell culture plate 2006 to inject MBs solution. The MBs injection was controlled by a syringe pump 2008 (e.g., NE-1010, New Era Pump Systems, Inc. NY, USA) with an infusion speed of 0.1 mL/min. Then, cells were sonicated for 30 s with a 5% duty cycle. Various sonication parameters including peak negative pressure (PNP) (0.1-0.7 MPa), and cycle number (CN) (20-2000 cycles) were tested.

After sonication, tested cells were incubated in a CO₂ incubator at 37° C. for 24 hours. To evaluate the sonoporation efficiency, cells were harvested from each well and were lysed in lysing buffer. Luciferase activity was measured by adding luciferin using a luminometer (e.g., GloMax, Promega Corporation, WI, USA), and reported in relative light units (RLU).

In this work, six independent experiment runs (i.e., n=6) were conducted for each group. Luciferase activity was compared among control and experiment groups using one-way analysis of variance (ANOVA). P-value<0.05 was considered statistically significant.

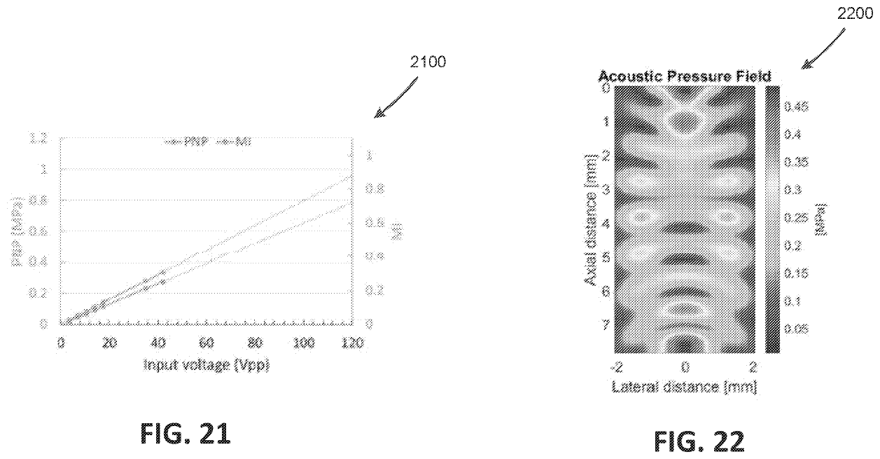

Measurement of the PNP and mechanical index (MI) for the prototyped 800 kHz catheter US transducer is illustrated in table 2100 of FIG. 21, indicating that the fabricated transducer can generate the required PNP (˜0.5 MPa) for sonoporation application. In addition, the focal length of the catheter transducer was ˜1 mm close to the transducer surface.

The simulated ultrasound beam (beam 2200 of FIG. 22) shows that the standing waves induced by the bottom wall of the cell culture plate can increase the acoustic pressure and deform the acoustic pressure field. The maximum acoustic pressure was creased by 20%, which was 0.48 MPa in the cell culture plate. Although the catheter US transducer has a ˜1 mm focal length, it can be observed that a high-acoustic-pressure area occurred near the bottom wall of the cell culture plate due to US wave reflection. This phenomenon indicates that cells can be placed on the bottom surface of each well for effective cellular sonication.

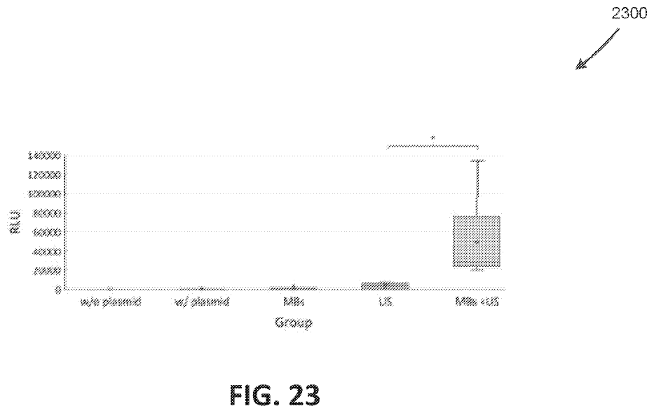

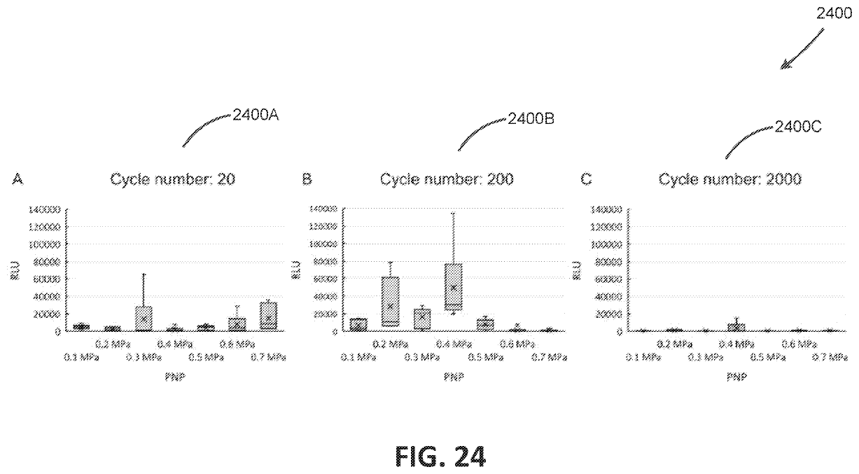

The negative control group and experiment group were compared in terms of luciferase activity (see series 2300 of FIG. 23). The negative control group consists of “w/o plasmid” group, “w/plasmid” group, “MBs only” group, and “US only” group and the experiment group refers to “MBs+US” group. Also, a positive control for gene transfer with lipofectamine was run in tandem to make sure the assay worked well (data not shown). For the sonication parameters, the “US only” group had a PNP of 0.2 MPa, and CN of 20 cycles, while the “MBs+US” group had a PNP of 0.4 MPa, and CN of 200 cycles. The luciferase assay (table 2300 of FIG. 23) demonstrated that the “w/plasmid” group only had a 102 level of RLU, while the “MBs+US” group achieved a 105 level of RLU. The corresponding maximum fold change of luciferase activity was approximately 1500-fold, indicating a significantly enhanced transfection. Following that, a preliminary parameter sensitivity study was conducted to investigate how PNP and CN affect sonoporation efficiency. FIG. 24 (which illustrates luciferase activity for sonoporation tests under various sonication parameters including PNP: 0.1-0.7 MPa, and CN: 20-2000 cycles. (n=6)) shows that 20 (table 2400A) or 200 cycles (table 2400B) are superior to 2000 cycles (table 2400C), and the optimal PNP ranges from 0.3 MPa to 0.5 MPa. According to FIG. 24, it was speculated that over-high acoustic pressure and over-long pulse duration (PD, which equals CN/US frequency) could induce cell death, and decrease sonoporation efficiency. While for FIG. 24, given that the PD of 20 cycles 800 kHz US is only 25 μs, the bubble-cell contact probability was relatively low during such a short period. Thus, the trend of PNP-RLU was different from table 2400B and 2400C of FIG. 24. In addition, it was interesting to see that 0.3˜0.4 MPa had a relatively low RLU for the 20 cycles condition (table 2400A), and 0.3 MPa has a relatively low RLU for the 200 cycles condition (table 2400B). It was speculated that it might be related to the conversion from stable cavitation and inertial cavitation. As a matter of fact, inertial cavitation can form larger pores in the cell membrane to achieve higher sonoporation efficiency, but it also collapsed the MBs suddenly. This might be able to explain why sonoporation efficiency goes down when the PNP is higher than 0.3 MPa in table 2400A and 0.2 MPa in table 2400B. After that, inertial cavitation plays the role, and sonoporation efficiency goes up with PNP increasing until cell death occurred.

Thus, the results for the developed 800 kHz forward-looking catheter US transducer showed that it was promising for intracorporeal sonoporation-induced drug/gene delivery in intratumoral immunotherapy.

### Example 6-Modification of the Tumor Microenvironment with M-HIFU|_{[HJA(6]}

Triple-negative and human ErbB-2 (HER2)-positive breast cancers (BCs) have a high rate of metastatic spread despite initial localized presentations and multimodality therapy. Cancer immunotherapy in the form of immune checkpoint blockade (ICB) has had modest activity limited to small percentages of triple-negative BCs. The reasons for the limited efficacy of ICB therapy in BC include a relatively low somatic mutation rate, the failure of the tumor to attract an immune infiltrate, particularly tumor-infiltrating lymphocytes, expression of additional immune checkpoint molecules in the tumor microenvironment (TME) suppressing the adaptive immune response, and suppression of intratumoral innate immunity by inhibitory cell type, such as regulatory T cells (Tregs), tumor-associated macrophages (TAMs), and myeloid derived suppressor cells. In fact, aggressive BC growth and invasion have been associated with TAM in both preclinical and clinical studies. Consequently, modifying the polarization of TAM in the TME has become a focus of attempts to increase the efficacy of BC immunotherapy. Unfortunately, pharmacological strategies to specifically modulate TAMs, without systemic alteration in non-TAM, have not been successful and alternatives such as local delivery or ablation are being considered.

Locally ablating tumor cells in the TME by image-guided delivery of various energies, including high-intensity focused ultrasound (HIFU), radiofrequency, microwaves, and cryoprobes has been clinically used as a minimally invasive therapy for localized prostate, breast, liver, kidney, bone and brain tumors. Such ablative therapies produce tumor cell destruction using different sources of energy but also elicit antitumor immune response against antigens within the tumor debris in situ. Conventional HIFU (thermal high-intensity focused ultrasound (T-HIFU)) induces rapid coagulative necrosis of the tissue at the targeted foci of applied energy. Tissue proximal to the targeted foci, while not coagulated, undergoes thermal stress sufficient enough to cause apoptosis. As T-HIFU-treated cells release endogenous danger signals, resulting in secretion of interferon gamma (IFN-γ) and/or tumor necrosis factor alpha (TNF-α) from immune cells, an increase in accumulation of CD4+ and CD8+ cells in T-HIFU-treated tumors has been observed. Nonetheless, limitations to the efficacy of T-HIFU in larger tumors have led to the study of alternative forms of HIFU to destroy tumors, such as high-pressure bursts that cause acoustic cavitation that have been termed as mechanical high-intensity focused ultrasound (M-HIFU).

Compared with T-HIFU, M-HIFU increased the accumulation of dendritic cells in treated tumors, demonstrated stronger antitumor efficacy, generated enhanced antitumor immunity, and reduced the risk of metastasis. It is noted that M-HIFU also increased the infiltration of T cells in the treated tumors. To better delineate the mechanism for the greater antitumor immunity induced by M-HIFU, single-cell RNA sequencing of the tumor was used and TME following either no treatment or conventional T-HIFU and M-HIFU. Changes in the local and distant TMEs of murine BCs were observed and a switch in macrophage subtype within M-HIFU-treated tumors that was absent following T-HIFU treatment was also observed. Further, upregulation of immune checkpoint molecules, such as programmed cell death-1 (PD-1)/programmed cell death ligand 1 (PD-L1), lymphocyte activation gene 3 or TIM-3 was observed, which could exhaust activated T cells in the TME, resulting in the suppression of antitumor immunity. Therefore, the combination of M-HIFU and anti-PD-L1 therapy was studied, which demonstrated upregulated gene expression by CD8+ T cells in type I interferon-mediated signaling pathway, T-cell proliferation, and chemokine/cytokine secretion, and was associated with dramatic increases in the local and distant antitumor effects, including complete responses in the majority of treated animals.

MM3MG, a murine premalignant mammary epithelial cell line, was transduced with the human HER2 oncogene by retroviral vectors and polybrene to express HER2 (referred to as MM3MG-HER2 cells) in our laboratory. JC cells, a murine BC cell line, were transduced with human HER3 gene (referred to as JC-HER3). 28 4T1-HER2 cells were obtained as well.

The VIFU 2000 system (Alpinion Medical Systems, Bothell, Washington, USA) was used for HIFU treatment. Cells or tumors were treated using a 1.5 MHz HIFU transducer under two different protocols (50% duty cycle, 1 Hz pulse repetition frequency, 20 W, 10 s or 2% duty cycle, 5 Hz pulse repetition frequency, 200 W, 20 s) to produce either thermal necrosis or mechanical lysis of the tumor cells. The former was defined as T-HIFU, and the latter was defined as M-HIFU. T-HIFU increased the temperature inside tumor tissues to >60° C. in a few seconds, while the temperature inside tumor tissue was <42° C. during M-HIFU. M-HIFU is similar to boiling histotripsy, which produces cavitation activities in vivo that may damage tumor tissue and cells through shear stresses generated by the complex bubble oscillation and bubble-bubble-tissue-cell interactions. The −6 dB focal dimension of the HIFU transducer was measured at a low-power level of 10 W to be 0.72 mm×7.22 mm in the lateral and axial directions, respectively. At the high-power levels used for T-HIFU (20 W) and M-HIFU (200 W), the corresponding focal dimensions based on numerical simulations were estimated to be 0.74 mm×6.50 mm and 0.60 mm×5.69 mm, respectively. Concerning focus for the treatment, both X and Y axis intervals were 2 mm, and a total of 9 points were selected in both in vitro and in vivo studies. A shorter interval of 1 mm was used only when tumors did not have enough size to put 2 mm for all intervals. The same spacing strategy was used for both M-HIFU and T-HIFU. To avoid skin or bone damage by HIFU treatment, tumor tissue just beneath the skin or close to thighbone was spared from exposure of focused ultrasound; thus, approximately 20%-40% of tumor tissues were ablated by HIFU treatments based on macroscopic assessment.

Female BALB/c mice or SCID-beige mice 5-8 weeks old (Jackson Labs, Bar Harbor, Maine, USA) were bred and maintained. Human HER2-transgenic mice were also utilized. F1 hybrid HER2 transgenic mice were established by crossing with BALB/c mice. Human HER3-transgenic mice (MMTV—neu/MMTV hHER3) with FVB background were also used. FVB mice homozygous for the hHER3 gene were established at Duke University and then crossed with BALB/c mice for establishment of BALB/c homozygous for the hHER3 gene.

Mice were euthanized when the local tumor volume reached 2000 mm³. To test the immunogenicity of HIFU-treated tumor cells, BALB/c mice received intradermal injections of in vitro T-HIFU-treated or M-HIFU-treated MM3MG-HER2 cells (1×106 cells) into the back on days −14 and −7. On day 0, some mice were euthanized and spleen, draining lymph nodes, and blood were collected for in vitro assays: IFN-γ ELISpot, flow cytometry, and cell-based ELISA. Other mice (10 mice/group) were inoculated with 1×106 MM3MG-HER2 cells into the left leg. Tumor size was measured serially and tumor volumes were calculated using the formula long axis×(short axis) 2×0.5.

For the therapeutic models using MM3MG-HER2 tumors, MM3MG-HER2 cells were subcutaneously inoculated into the left leg (1×106 cells) of the mice on day 0. In the bilateral tumor model, 1×105 or 5×105 cells were also inoculated into the right flank on day 0. Established leg tumors were treated with T-HIFU or M-HIFU on day 7. For the rechallenge experiment, mice cured by M-HIFU treatment received a subcutaneous injection of MM3MG-HER2 cells (1×106 cells) into the flank on day 35 (28 days after M-HIFU) and tumor size and mouse survival were monitored. For the combination treatment with anti-PD-L1 antibody, mice received peritoneal injection of 100 μg anti-PD-L1 antibody (clone 10F.9G2, Bio X Cell, West Lebanon, NH) or Isotype control IgG (clone LTF-22, Bio X Cell) on days 12, 15 and 18 in unilateral tumor models, or on days 10, 13 and 16 in bilateral tumor models. For JC-HER3 tumor model, cells were inoculated into the left leg (1×106 cells)±inoculation into the right flank (5×105 cells) on day 0. Leg tumors were treated with M-HIFU on day 8 (for combination treatment experiments) or 11 (for single M-HIFU treatment experiments). For the combination treatment, mice received intraperitoneal injection of anti-PD-L1 antibody or Isotype control IgG (200 μg/injection) on days 8, 11, 15 in unilateral tumor models, or on days 8, 11, 15 and 18 in bilateral tumor models. For depletion of immune cells, mice received peritoneal injection of 250 μg antibody against CD4 (clone GK1.5, Bio X Cell), against CD8a (clone 53-6.72, Bio X Cell) or 10 μL antibody against natural killer (NK) cells (anti-Asialo GM1 antibody: Wako Pure Chemical Corporation, Osaka, Japan) 1 day before the first HIFU treatment and 2 days after the first HIFU treatment, followed by injection of the same amount every 5 days throughout experiments.

Mouse IFNγ-ELISpot assays (e.g, Mabtech, Cincinnati, Ohio, USA) were performed according to the manufacturer's instructions. Cells were stimulated with HER2 intracellular domain (ICD) peptide, HER2 extracellular domain (ECD) peptide (25 μg/mL; JPT Peptide Technologies, Berlin, Germany) or irrelevant HIV-gag eptide mix (2.6 μg/mL, JPT Peptide Technologies). The number of IFN-γ spots was counted with a high-resolution automated ELISpot reader system (e.g., Carl Zeiss, White Plains, New York, USA) using the KS ELISpot V.4.2 software.

Plates were coated with 3×104 4T1 parental cells or 6×104 4T1-HER2 cells per well overnight. A serial dilution of serum (final titrations 1:50-1:6400) was added, incubated for 1 hour on ice. The plates were washed and fixed with 1% formalin, followed by incubation with IRDye 800CW Donkey anti-mouse (1:2000; LI-COR Biosciences, Lincoln, Nebraska, USA) for 60 min. Fluorescence intensity was determined using an Odyssey CLx LI-COR reader (LI-COR) using the 800 nm channel.

Tumors were weighed and homogenized in Cell Lysis Buffer (Cat #9803, Cell Signaling, nine times volume of tumor weight) with added PMSF (1 mM) using Qiagen TissueRupter (e.g., Qiagen, Germantown, Maryland, USA) for up to 20 s on ice. Tumor homogenates were then sonicated using Branson Ultrasonic SLPe Digital Sonifier Cell Disruptor (e.g, Branson Ultrasonics, Danbury, Connecticut, USA) for 10 s on ice. Sonicated samples were centrifuged at 13 000 rpm at 4° C. for 20 min, and collected supernatants were used as tumor lysates for ELISAs. Tumor lysates were assessed for the level of IFN-γ, TNF-α and transforming growth factor beta 1 (TGF-β1) using commercially available ELISA kits for IFN-γ (Cat #ab46081; Abcam, Cambridge, Massachusetts, USA), TNF-α (BMS607-3; Invitrogen, Waltham, Massachusetts, USA) and TGF-β1 (BMS608-4, Invitrogen), and assays were performed according to the manufacturers' instructions.

Single-cell suspensions of tumor tissue were obtained by manually disrupting tumor using a razor followed by enzymatic digestion. Single-cell suspensions of spleen and lymph nodes were obtained by manual mashing and filtration through a 70 μm cell strainer (BD Biosciences, San Jose, California, USA). Cell were stained using LIVE/DEAD Fixable Aqua Dead Cell Stain Kit (e.g., Thermo Fisher Scientific, Rockford, Illinois, USA), then stained with surface marker antibody (online supplemental table 1) for 30 min, at room temperature. Isotype IgG or fluorescence minus one (FMO) controls were used as negative staining controls. Anti-CD16-32 antibody (Thermo Fisher) was used to block FcγIII/II receptor. Intracellular staining was carried out using Fixation/Permeabilization and Permeabilization Buffer (Thermo Fisher) following the manufacturer's instructions. Stained cells were acquired on an LSRII flow cytometer (BD Biosciences) and analyzed using FlowJo software IX (BD Biosciences).

Single-cell suspensions dissociated from untreated or HIFU treated tumor (8 days after HIFU treatment) were obtained as described above. For combination therapy experiments, mice received intraperitoneal injections of 100 μg anti-PD-L1 antibody or isotype control IgG antibody both 3 and 6 days after HIFU treatment. CD45+ leukocytes were sorted from the tumor digest by flow cytometry, and a cDNA library was prepared using Bio-Rad single-cell isolator (ddSEQ) and SureCell WTA 3′Library Prep kits from Illumina. Raw sequencing data was generated in the form of FastQ files which were uploaded to a BaseSpace sequencing Hub (Illumina, San Diego, California, USA) for expression quantification using an automated pipeline. The resulting gene expression matrix files for each treatment condition were analyzed using Partek Flow software (Partek, St. Louis, Missouri, USA). Unsupervised clustering was done to separate the cell types and markers for the cell types were identified using differential gene expression. These markers were then used for identifying the cell subpopulations within the CD45+ sorted immune cells. The preprocessed gene counts were used to generate Uniform Manifold Approximation and Projection (UMAPs) for visualization of the cell types in different treatment conditions. Gene Ontology (GO) enrichment analysis, Kyoto Encyclopedia of Genes and Genomes (KEGG) pathway analysis, and differential gene expression analysis were done using Partek Flow software.

Staining of formalin-fixed paraffin-embedded tumor tissue sections was performed using rabbit-anti-mouse CD38 antibody (1:150, D4V8L; Cell Signaling Technology, Danvers, Massachusetts, USA), CD4 antibody (1:100, D7D2Z; Cell Signaling) or CD8a antibody (1:400, D4W2Z; Cell Signaling) by the horseradish peroxidase (HRP) method. Antigen retrieval was performed with Citrate Unmasking Solution (Cell Signaling). Before incubation with HRP (Histofine Simple Stain Mouse MAX PO (R), Nichirei Biosciences, Tokyo, Japan), DAB Substrate Kit (Vector Labs, Burlingame, California, USA) was used for visualization of signal. Isotype-matched rabbit IgG was used as a negative staining control. Stained slides were scanned on a DP80 microscope (OLYMPUS, Tokyo, Japan) and digital images were viewed using cell-Sens (OLYMPUS).

Data are presented as mean±SEM in tumor growth graphs, or as mean±SD for in vitro assays and flow cytometry data. Tumor volumes, flow cytometry, ELISA, and ELISPOT data from experiments with three or more treatment groups were analyzed by one-way analysis of variance with Tukey's multiple comparisons test. A two-tailed, unpaired Student's t-test was used for experiments with only two groups. Tumor volumes were analyzed at the terminal endpoint only, unless otherwise indicated. Statistical analyses were performed using Prism (e.g., GraphPad, San Diego, California, USA). Kaplan-Meier survival curves for tumor-bearing mice were generated and log-rank tests were performed using JMP Pro V.11.0 software (e.g., SAS Institute, Cary, North Carolina, USA). P values of 0.05 or less were considered statistically significant. Not all significant differences are shown in every graph (*p<0.05, **p<0.01, ***p<0.001).

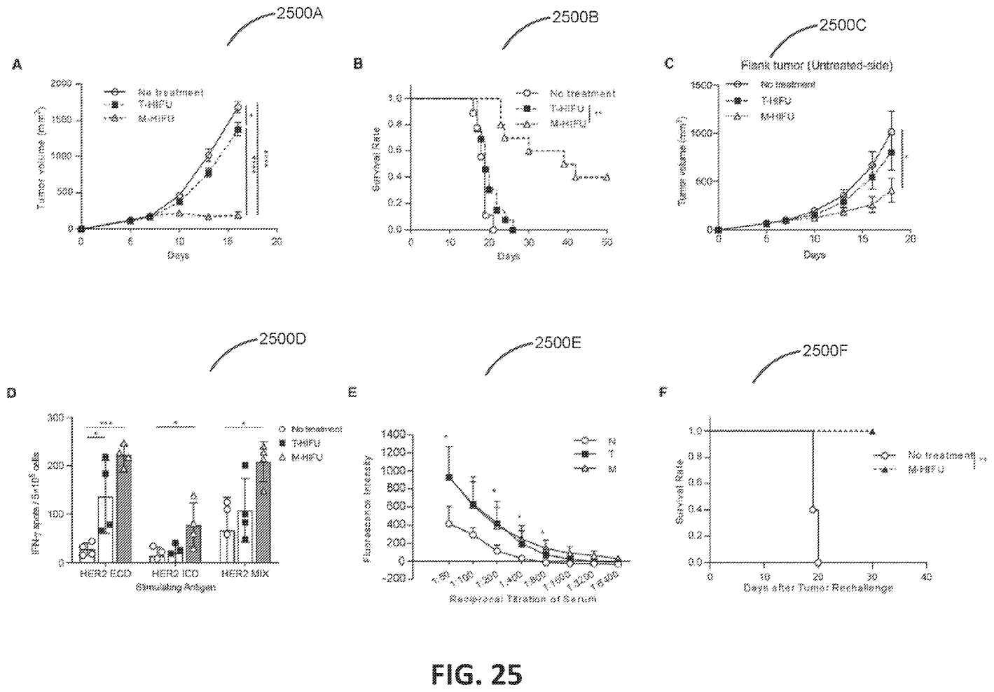

Implanted MM3MG-HER2 BCs in BALB/c mice were treated with either M-HIFU or T-HIFU and the antitumor effect was assessed on both the local (treated) and distant tumor mass. There was greater control of the treated tumors as well as untreated distant tumors with M-HIFU compared with T-HIFU (tables 2500A-2500C of FIG. 25). Tumor growth suppression of both the treated and untreated disease sites by M-HIFU was confirmed in two other murine BC models, E0771-OVA and JC-HER3. M-HIFU could induce significantly stronger cellular immune responses for HER2 ECD, HER2 ICD and mixed peptide antigens when compared with untreated control, while T-HIFU could induce only mildly stronger response for HER2 ECD antigen (table 2500D of FIG. 25). There was a clear trend for stronger cellular immune responses induced by M-HIFU compared with T-HIFU. However, M-HIFU and T-HIFU induced similar levels of humoral immunity against HER2 expressing tumor cells (table 2500E of FIG. 25). Also in JC-HER3 tumor model, the induction of HER3 antigen-specific cellular immune response in HER3 transgenic mice was confirmed after M-HIFU treatment, suggesting the strong capacity of M-HIFU for the induction of antitumor immunity by breaking tolerance. Importantly, there was a long-lived immune memory response induced in mice that had previously rejected their tumor following M-HIFU treatment (table 2500F of FIG. 25). This memory response could not be measured in T-HIFU-treated mice as none of the treated tumors fully regressed (table 2500B of FIG. 25). A potential explanation for this difference in antitumor response is a baseline difference in the immunogenicity of tumor cells following treatment with M-HIFU compared with T-HIFU. To test this hypothesis, mice were vaccinated with in vitro HIFU-treated MM3MG-HER2 cells and assessed their immunogenicity. Despite a difference in the type of cell death induced by M-HIFU versus T-HIFU (apoptosis vs necrosis), there was no significant difference in the systemic T-cell activation, generation of tumor antigen-specific antibodies, or tumor control elicited by inoculation with M-HIFU or T-HIFU killed tumor cells.

With respect to FIG. 25, tables 2500A-2500F depict data regarding superior growth suppression of local and distant tumors and enhanced tumor antigen-specific cellular immune responses by M-HIFU compared with T-HIFU. For table 2500A, 1×106 MM3MG-HER2 cells were injected into the legs of BALB/c mice Established leg tumors were treated with M-HIFU or T-HIFU on day 7 after tumor inoculation. A comparison of tumor growth curves thereof is shown. Table 2500B depicts survival curves. Mice were euthanized when tumor volume reached 2,000 mm³ or on day 50. n=9 mice (no treatment), 10 (M-HIFU) or 13 (T-HIFU) (A,B), 32 mice in total. Log-rank test was performed. For the data of table 2500C, MM3MG-HER2 cells were injected into the left leg (1×106 cells) and the right flank (1×105 cells) of the HER2 transgenic mice on day 0. Leg tumors were treated with M-HIFU or T-HIFU on day 7. Comparison of flank tumor growth curves is shown. n=13 mice (no treatment) or 14 (M-HIFU and T-HIFU), 41 mice in total. For the data of table 2500D, lymphocytes were isolated from the spleen on day 18 after tumor inoculation (11 days after HIFU treatment), and IFN-γ secretion was detected by an ELISpot assay. Average values of spot numbers for HER2 peptide of ECD, ICD and mix (ECD+ICD) are shown. n=4 per group. For the data of table 2500E, serum was collected from mice on day 11 after HIFU treatment. The levels of anti-HER2 antibody in the serum of mice were evaluated with cell-based ELISA. n=3 mice (no treatment) or 8 (M-HIFU and T-HIFU), 19 mice in total. For the data of FIG. 4500F, mice cured from MM3MG-HER2 tumor by M-HIFU treatment were rechallenged with subcutaneous injection of MM3MG-HER2 cells (1×106 cells/mouse) 28 days after M-HIFU treatment. Age-matched naive female BALB/c mice were used as a control group. The survival rate of mice is shown and log-rank test was performed. n=5 mice per group, 10 mice in total. (A-C) Error bars represent SE. (D,E) Error bars represent SD. *P<0.05, **P<0.01, ***P<0.001, ****P<0.0001. ECD, extracellular domain; HER2, human ErbB-2; ICD, intracellular domain, IFN-γ, interferon gamma; M-HIFU, mechanical high-intensity focused ultrasound; T-HIFU, thermal high-intensity focused ultrasound.

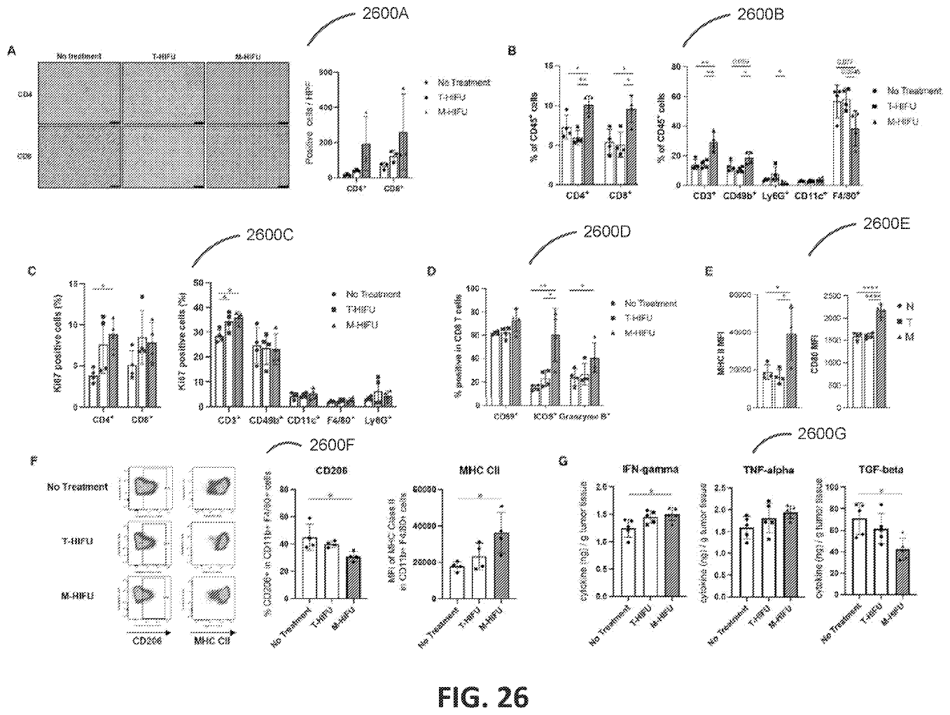

Because tumor cells treated in vitro with M-HIFU or T-HIFU were similarly immunogenic, it was hypothesized that differential modification of the TME by the two HIFU treatments was the cause of differences in the induction of systemic antitumor immunity, and thus efficacy, in the BC models. What was observed was greater CD4 and CD8 T-cell infiltration in M-HIFU-treated tumors and slightly increased T-cell infiltration in T-HIFU-treated tumors compared with no treatment controls by immunohistochemistry (plot 2600A of FIG. 26). This was confirmed by flow cytometry of digested tumors which demonstrated an increase in both CD4 and CD8 T cells as well as NK cells following M-HIFU treatment (plot 2600B of FIG. 26). To confirm the modification of immune cell profile, absolute numbers of tumor-infiltrating immune cells were determined for each treatment group. Increased numbers of CD4 T cells, CD8 T cells, and NK cells were confirmed in M-HIFU-treated tumors, while the differences in granulocytes and macrophage populations were not observed. Enhanced proliferation of T cells was seen in M-HIFU-treated tumors compared with control tumors by Ki67 staining (plot 2600C of FIG. 26). In addition to an increase in infiltration and active proliferation, the CD8 T cells present in M-HIFU-treated tumors had increased expression of T-cell activation markers and granzyme (plot 2600D of FIG. 26). In M-HIFU-treated tumors, dendritic cell (DC) maturation was increased as evidenced by upregulation of Major Histocompatibility Complex (MHC) class II and CD80 (plot 2600E of FIG. 26). Importantly, M-HIFU affected the polarization status of tumor-infiltrating macrophages as shown in plot 2600F of FIG. 26. Positivity of CD206 expression was significantly lower and expression level of MHC class II was significantly higher in macrophages in M-HIFU-treated tumors compared with those in control tumors, suggesting that M-HIFU can induce repolarization of macrophages and make more antitumor-TME. To support this finding, significantly lower TGF-β1 levels and higher IFN-γ levels were observed in M-HIFU-treated tumors compared with control tumors (table 2600G of FIG. 26).

With respect to FIG. 26, enhanced intratumoral infiltration of activated CD4+ and CD8+ cells and M1 polarized macrophages by M-HIFU. 1×106 MM3MG-HER2 cells were injected into legs of BALB/c mice. Established leg tumors were treated with M-HIFU or T-HIFU on day 7 after tumor inoculation. With respect to plot 2600A, immunohistochemical staining of T cells in tumor sections. Tumors on day 13 after HIFU treatment were collected, fixed with formalin and stained with anti-mouse CD4 and CD8 monoclonal antibodies. Representative images are shown (left: no treatment, middle: T-HIFU, right: M-HIFU). Scale bar is 50 μm. Quantification of positive cells in high-power field (HPF) is shown in the right panel. Error bars represent SD, n=3 per group. Regarding plot 2600B, seven days after HIFU treatments of MM3MG-HER2 tumors in mice, tumors were collected and digested for flow cytometry analysis. The percentages of CD4+, CD8+, CD3+, CD49b+, Ly6G+, CD11c+, and F4/80+ cells in alive CD45+ cells were analyzed for each HIFU treatment group. n=4 per group. Regarding plot 2600C, the expression of proliferation marker Ki67 was analyzed for each cell type in tumor-infiltrating immune cells by flow cytometry, and percentages of Ki67 positive for each cell type are shown. n=4 per group. Regarding plot 2600D, the expression of CD69+, Inducible T-cell costimulator (ICOS)+, and granzyme B+ by CD8+ cells were analyzed by flow cytometry and shown for each HIFU treatment group. n=4 per group. Regarding plot 2600E, MFI of MHC class II (left) and CD80 (right) expression on CD11c+ dendritic cells are shown for each HIFU treatment group. Regarding plot 2600F, expression of CD206 and MHC class II by CD11b+F4/80+ macrophage population was analyzed for each treatment group. Representative dot plots of CD206 and MHC class II staining are shown in the left panel. Percentages of CD206-positive macrophages and mean fluorescence intensity of MHC class II expression are shown. n=4 per group. Regarding plot 2600G, ELISAs for IFN-γ, TNF-α, and TGF-β1 were performed with tumor lysates made from MM3MG-HER2 tumors treated with no treatment, T-HIFU or M-HIFU. n=5 per group. Error bars represent SD. *P<0.05, **P<0.01, ****P<0.0001. IFN-γ, interferon gamma; MFI, mean fluorescent intensity; M-HIFU, mechanical high-intensity focused ultrasound; TGF-β1, transforming growth factor beta 1; T-HIFU, thermal high-intensity focused ultrasound; TNF-α, tumor necrosis factor alpha.

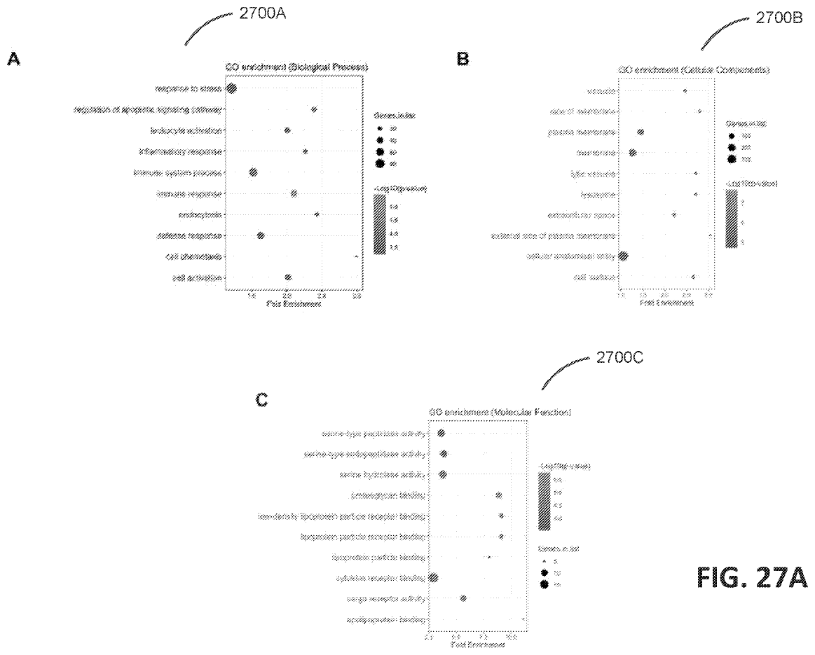

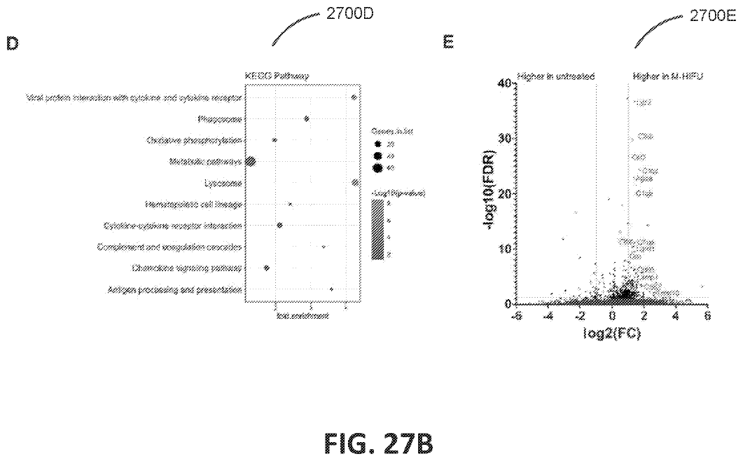

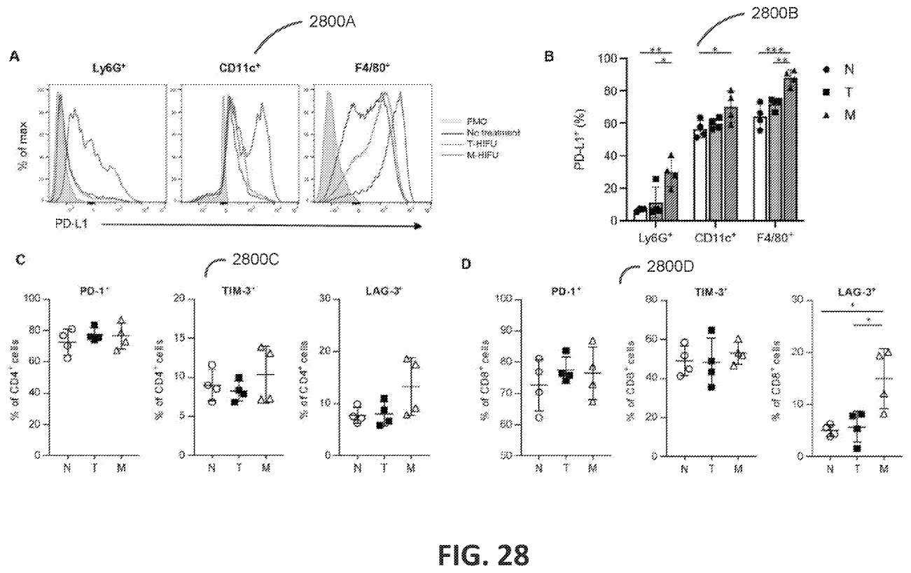

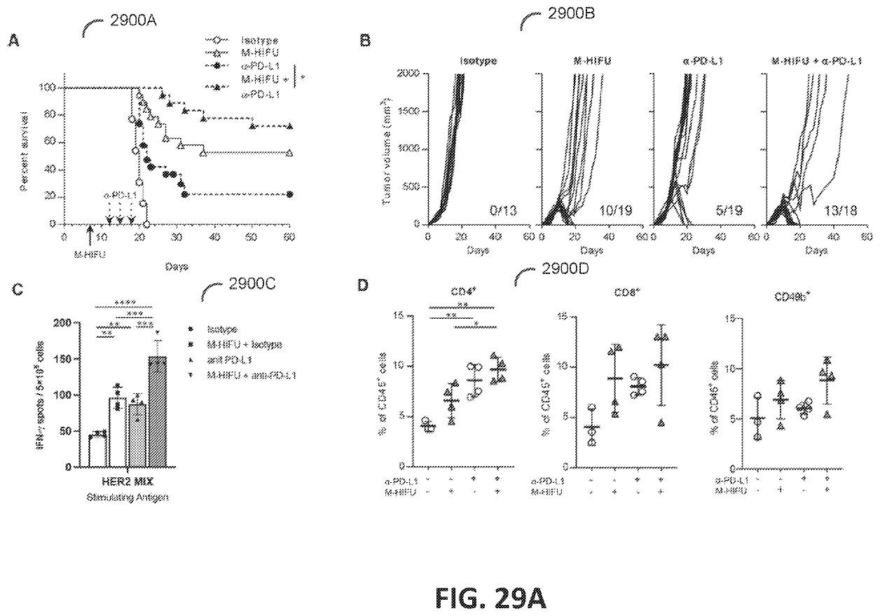

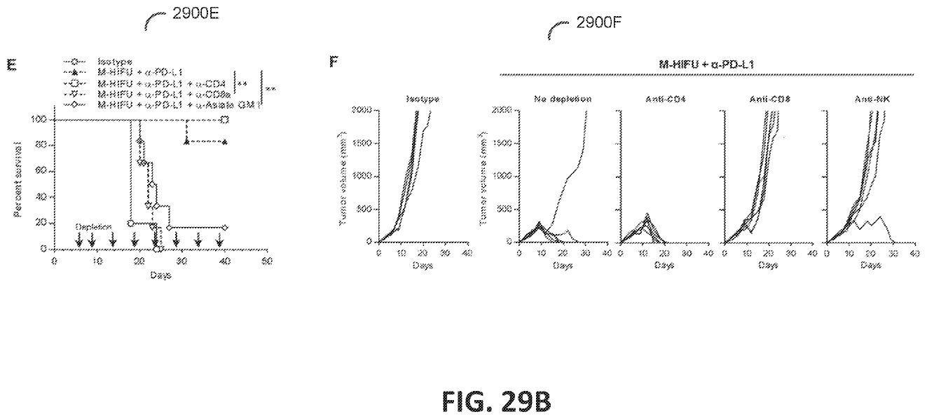

To study the effect of HIFU on the TME in a more detailed fashion, a single-cell RNA sequencing (scRNA-seq) analysis was conducted on tumor-infiltrating leukocytes from untreated tumors to compare with the gene expression from tumors treated with M-HIFU and T-HIFU. A GO enrichment analysis and KEGG pathway analysis of differentially expressed genes (DEGs) within the identified macrophage populations from the two HIFU strategies was performed and compared with macrophages in the untreated tumors. Ten of the significantly upregulated GO terms of the biological process category in M-HIFU-treated macrophages are shown in table 2700A of FIG. 27A. Upregulated genes were mainly involved in immune response, inflammatory response, leukocyte activation, immune system process, cell chemotaxis, endocytosis and regulation of apoptotic signaling pathway. A similar analysis of the top 10 differently expressed GO terms in T-HIFU-treated tumors did not contain any immune-related terms but rather centered around biosynthetic processes.

The top 10 GO terms of the cellular components and molecular function categories for both M-HIFU-treated and T-HIFU-treated tumors compared with untreated tumors are shown in plots 2700B and 2700C of FIG. 27A. There is no overlap in the top terms that are upregulated following these different forms of HIFU, again demonstrating fundamental differences in the molecular consequences of each therapy. KEGG pathway analysis of M-HIFU-treated tumors shows that the top 10 upregulated pathways are enriched for antigen processing/presentation, cytokine-cytokine receptor interaction, chemokine signaling pathways, and phagosome/lysosome pathways, consistent with an inflammatory, antitumor phenotype of macrophages after M-HIFU (plot 2700D of FIG. 27B). Tumors treated with T-HIFU only had seven KEGG pathways that were significantly upregulated compared with untreated tumors. These pathways were all involved in metabolic processes. The differences in significantly upregulated pathways between M-HIFU-treated and T-HIFU-treated tumors highlight the immunogenic nature of our M-HIFU therapy at this timepoint. Differential gene expression analysis of tumor-infiltrating macrophages identified 298 genes that were expressed at statistically higher levels (FDR adjusted p value <0.05) than untreated tumors (plot 2700E of FIG. 27B). A number of genes (Irg1, Ccl2, Maff, Ier3, Lep2, Ptpn2, Cd14, Cxcl16, Ier3, Nfkbia, Ccr12, and Tlr2) are signature genes for M1/classically activated macrophages, 34 and other genes (Tnfsf10Ctsc, Gzme, Cd8a, and Pdcd1) are commonly associated with M1 macrophages. These findings confirm a highly activated M1-biased macrophage population within the TME of M-HIFU-treated tumors. Thus, scRNA-seq analysis demonstrated that M-HIFU induced an inflammatory, M1-biased macrophage population, which might be contributing to the enhanced antitumor immunity in M-HIFU-treated tumors.

With respect to FIGS. 27A and 27B, GO enrichment analysis and KEGG pathway analysis of DEGs in macrophages after M-HIFU treatment. With respect to plots 2700A-2700C, GO enrichment analysis of DEGs that are upregulated in macrophages derived from M-HIFU-treated tumors compared with macrophages from untreated tumors. Enrichment scores of GO terms are shown for the categories of (A) biological process (plot 2700A), (B) cellular components (plot 2700B), and (C) molecular function (plot 2700C). Regarding plot D, KEGG pathway analysis of DEGs that are upregulated in macrophages derived from M-HIFU-treated tumors compared with macrophages from untreated tumors. Top 10 KEGG pathways in terms of enrichment scores are demonstrated. Regarding plot 2700E, differential gene expression analysis in tumor-infiltrating macrophages from M-HIFU-treated tumors compared to those from untreated tumors. Representative genes that were significantly upregulated in macrophages from the M-HIFU-treated group (log 2 (fold Change)>1, FDR<0.05) are shown in red DEG, differentially expressed gene; FDR, false discovery rate; GO, Gene Ontology; M-HIFU, mechanical high-intensity focused ultrasound.

Despite productive antitumor immune responses in nearly half of M-HIFU-treated mice, many eventually developed progressive disease (tables 2500A and 2500B of FIG. 25). Thus, the etiology for this tumor escape was investigated, beginning by evaluating the expression of immune checkpoint molecules in M-HIFU-treated versus T-HIFU-treated tumors. At 7 days post-treatment, PD-L1 expression was increased on neutrophils (Ly6G+), DCs (CD11c+), and macrophages (F4/80+) only in M-HIFU-treated tumors (plots 2800A and 2800B of FIG. 28). Analysis at just 3 days after HIFU treatment, however, revealed that neither mode of HIFU treatment increased the PD-L1 expression in these tumor-associated cell populations. This suggests that HIFU itself does not cause immediate upregulation of PD-L1, but is instead the result of the ongoing immune response caused by M-HIFU treatment. IFN-γ, which was increased in tumor tissues on day 5 after M-HIFU treatment (plot 2600G of FIG. 26), might be playing an important role in this upregulation of PD-L1. Other immune checkpoint molecules including PD-1 and TIM3 were not significantly altered following M-HIFU, but lymphocyte activation gene 3 (LAG-3) expression on CD8+ cells was significantly higher after M-HIFU than T-HIFU (plots 2800C and 2800D of FIG. 28). Taken together, these data demonstrate enhanced antitumor immunity within the TME caused by M-HIFU that is not seen with T-HIFU but may be stifled by a concomitant increase in immune checkpoint molecules.

With respect to FIG. 28, plots 2800A-2800D illustrate enhanced expression of immune checkpoint molecules by tumor-infiltrating immune cells after M-HIFU treatment. Regarding plot 2800A, representative flow cytometry histograms showing PD-L1 expression on Ly6G+ cells, CD11c+ cells and F4/80+ cells are shown. Blue: M-HIFU, red: T-HIFU, black: no treatment, gray filled: isotype control. Regarding plot 2800B, percentages of PD-L1+ cells in Ly6G+ cells, CD11c+ cells, and F4/80+ cells among alive CD45+ tumor-infiltrating immune cells are shown for each HIFU treatment group. n=4 per group. Regarding plots 2800C and 2800D, expression of immune checkpoint molecules, PD-1, TIM-3 and LAG-3, on tumor-infiltrating CD4+ (plot 2800C) and CD8+ (plot 2800D) cells are shown. n=4 per group. Error bars represent SD. *P<0.05, **P<0.01, ***P<0.001. LAG-3, lymphocyte activation gene 3; M-HIFU, mechanical high-intensity focused ultrasound; PD-1, programmed death-1; PD-L1, programmed cell death ligand 1; T-HIFU, thermal high-intensity focused ultrasound.

Based on the upregulation of immune checkpoint molecules after M-HIFU, it was hypothesized that PD-1/PD-L1 blockade could enhance the antitumor efficacy of M-HIFU treatment. To test this, M-HIFU treatment was combined with anti-PD-L1 antibody administration. This combined treatment cured 72.2% of mice implanted with MM3MG-HER2 tumors compared with 26.3% and 52.6% when anti-PD-L1 or M-HIFU, respectively, were given alone (plots 2900A and 2900B of FIG. 29A). The combination also resulted in tumor eradication and long-term survival in 37.5% of mice using another BC model, JC-HER3. Analysis of IFN-γ ELISpot assay of splenocytes demonstrate a significant increase in the number of HER2-specific IFN-γ-secreting cells in mice treated with M-HIFU+anti-PD-L1 compared with those treated with M-HIFU or anti-PD-L1 alone (plot 2900C of FIG. 29A) flow cytometric analysis of digested tumors revealed that the proportion of CD4+ T cells, CD8+ T cells, and NK cells increased in tumors treated with combination therapy (plot 2900D of FIG. 29A), and enhanced activation of both CD4+ and CD8+ T cells in combination therapy. Stronger cytolytic activity of tumor-infiltrating CD8+ T cells was suggested in M-HIFU monotherapy and combination therapy based on enhanced granzyme B expression. Additionally, CD4+Foxp3+ Treg cells were increased in tumors treated with combination therapy compared with control tumors or tumors treated with M-HIFU monotherapy, although the difference was not statistically significant.

In order to demonstrate which infiltrating cells were responsible for the observed antitumor efficacy, MM3MG-HER2 tumors were treated with combination therapy in the presence of depleting antibodies against CD4+, CD8+, or NK cells. Depletion of CD8+ or NK cells abrogated the antitumor efficacy of the combination therapy, but depletion of CD4+ cells did not result in appreciable change in antitumor effect (plots 2900E and 2900F of FIG. 29B).

With respect to FIG. 29, M-HIFU and PD-L1 blockade synergize to reject local tumor. Regarding plot 2900A, established MM3MG-HER2 tumors in the legs of BALB/c mice were treated with M-HIFU on day 7. Anti-PD-L1 antibody (100 μg/100 μL) or isotype control IgG (100 μg/100 μL) was injected intraperitoneally 5, 8, and 11 days after M-HIFU treatment. Survival curves are shown and log-rank test was performed. n=13 mice (isotype control), 19 (monotherapy groups) or 18 (combination group), 50 mice in total. Regarding plot 2900B, individual tumor growth curves are shown for each treatment group. The numbers in each plot show mice with tumor eradication/mice in the group. Regarding plot 2900C, nine days after the initiation of M-HIFU treatment with/without anti-PD-L1 antibody, spleens were collected for immune assays. Induction of HER2 antigen-specific cellular response was analyzed by IFN-γ ELISpot assay using harvested splenocytes and HER2 peptide mix as a stimulating antigen. n=4 per each group. Regarding plot 2900D, the percentages of CD4+, CD8+, and CD49b+ cells in tumor-infiltrating CD45+ cells were analyzed by flow cytometry analysis. n=4 (treatment groups) or 3 (control group). Regarding plots 2900E and 2900F, mice were treated with the combination of M-HIFU and anti-PD-L1 antibody as in plot 2800A of FIG. 28, with or without administration of depleting antibody for CD4+, CD8a+ and NK cells on days 6, 9, 14, and every 5 days until the end of the experiment. n=5 mice (isotype control) or 6 (other groups), 29 mice in total. Regarding plot 2900E, survival curves are shown and log-rank test was performed. Regarding plot 2900F, individual tumor growth curves are shown for each cell depletion group. Error bars represent SD. *P<0.05, **P<0.01, ***P<0.001, ****P<0.0001. HER2, human ErbB-2; IFN-γ, interferon gamma; M-HIFU, mechanical high-intensity focused ultrasound; PD-L1, programmed cell death ligand 1.

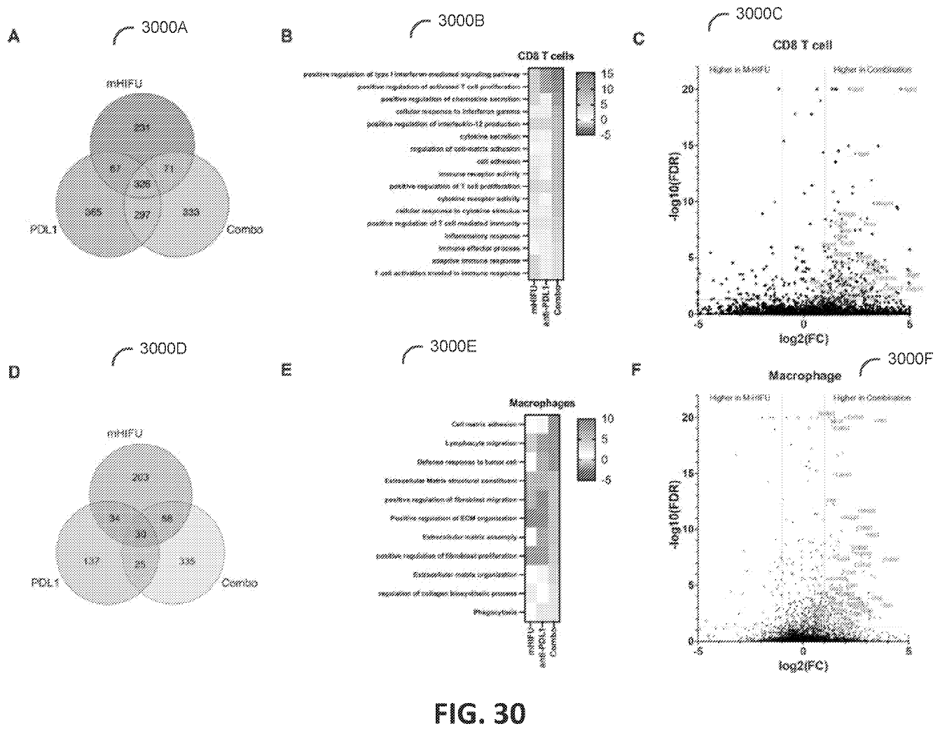

In order to explain the enhanced antitumor efficacy of the addition of anti-PD-L1 treatment, leukocyte populations were further characterized by conducting scRNAseq analysis on the tumor-infiltrating CD45+ immune cells derived from mice treated with no treatment, M-HIFU, a-PD-L1 alone or combination treatment. DEGs that were significantly upregulated in treatment groups compared with no treatment control were identified in CD8 T cells or macrophages, and the number of DEGs is shown in plots 3000A-3000F of FIG. 30. In CD8 T cells, 1690 DEGs were identified in the three treated groups, and 333 genes among them were unique to the combination treatment, while 231 genes and 365 genes were unique to M-HIFU and anti-PD-L1 monotherapy, respectively, indicating that the combination treatment exerts not a mere additive effect of monotherapies (plot 3000A of FIG. 30). The most significantly enriched GO terms of the upregulated DEGs in the combination treatment were shown as enrichment scores in the heatmap with other two treatments (plot 3000B of FIG. 30). In CD8 T cells, the DEGs mainly enriched in type I interferon-mediated signaling pathway, activated T-cell proliferation, chemokine secretion, cellular response to interferon gamma, interleukin (IL)-12 production, and cytokine secretion. KEGG pathway analysis indicates that upregulated DEGs were significantly enriched in pathways such as complement and coagulation cascades, cytokine cytokine receptor interaction, and NF-kappa B signaling pathway (online supplemental FIG. 10A). Thus, the combination treatment appears to activate CD8 T cells through regulation of multiple pathways and enhance antitumor immunity.

To clarify the genes involved in enhanced antitumor efficacy of combination treatment over M-HIFU monotherapy, gene expression levels by CD8 T cells in the combination treatment and M-HIFU monotherapy were compared (plot 3000C of FIG. 30). Genes related to CD8 T-cell activation, such as Cd33, Cx3cr1, Cxcl3, Cxcl11, and Cxcl16 were expressed more than twofold stronger following the combination treatment compared with M-HIFU monotherapy, confirming the enhanced activation of CD8 T cells by combining anti-PD-L1 to M-HIFU treatment, which corresponded to the superior antitumor efficacy against remote tumors observed in bilateral tumor models. In macrophages, among 822 DEGs significantly upregulated in the treated samples, 335 genes were unique to the combination treatment (plot 3000D of FIG. 30), mainly enriched in lymphocyte migration and defense response to tumor cells, as well as in the multiple pathways related to extracellular matrix/fibroblasts (plot 3000E of FIG. 30). KEGG pathway analysis revealed gene enrichment in protein digestion and absorption, extracellular matrix-receptor interaction, cell adhesion molecules, PI3K-Akt signaling pathway and T-cell receptor signaling pathway in the combination treatment. These results may suggest more active involvement of combination therapy-treated macrophages in the enhanced antitumor activity and remodeling of tissues after disruption of tumors by ultrasound. Interestingly, as shown in plot 3000F of FIG. 30, tumor infiltrating macrophages of the combination treatment group, when compared with those in M-HIFU treated tumors, showed relatively upregulated expression of genes signatures for both M1/classically activated macrophages (Den, Acpp, Csf1, Adgr12, and Col4a2) and M2/alternatively activated macrophages (Il1rl1, Mmp9, Mfsd6, Angptl2, Spint2, and Sft2d2).

With respect to FIG. 29, regarding combination of M-HIFU and anti-PD-L1 antibody induces upregulation of unique DEGs in tumor-infiltrating CD8 T cells and macrophages. Established MM3MG-HER2 tumors in the legs of BALB/c mice were treated with M-HIFU, followed by intraperitoneal injections of 100 μg anti-PD-L1 antibody or isotype control IgG antibody both 3 and 6 days after HIFU treatment. Alive CD45+ leukocytes were isolated from enzymatically digested tumors by flow-based sorting and used for scRNA-seq. Data analysis was performed using Partek Flow software. Regarding plot 3000A, the numbers of DEGs of CD8 T cells that were upregulated in treatment groups compared with no treatment control are shown in the Venn diagram. Regarding plot 3000B, GO enrichment analysis was performed for the upregulated DEGs in CD8 T cells in each treatment group and summarized in the heatmap. Top 17 GO terms in combination treatment are shown together with enrichment data in M-HIFU and anti-PD-L1 monotherapy group. Regarding plot 3000C, differential gene expression analysis was performed for upregulated DEGs in CD8 T cells between combination treatment and M-HIFU monotherapy. Representative DEGs that are significantly upregulated (log 2(fold change)>1, FDR<0.05) in the combination group are shown in red letters. Regarding plot 3000D, the numbers of DEGs of macrophages that were upregulated in the treatment groups compared with no treatment control are shown in the Venn diagram. Regarding plot 3000E, GO enrichment analysis was performed for the upregulated DEGs in macrophages in each treatment group and summarized in the heatmap. Top 11 GO terms in the combination treatment are shown together with enrichment data in the M-HIFU and anti-PD-L1 monotherapy group. Regarding plot 3000F, differential gene expression analysis was performed for upregulated DEGs in macrophages between combination treatment and M-HIFU monotherapy. Representative DEGs that are significantly upregulated (log 2(fold change)>1, FDR<0.05) in the combination group and contributed to the activated KEGG pathways or are known as M1 or M2 signature genes are shown in red letters. DEG, differentially expressed gene; GO, Gene Ontology; M-HIFU, mechanical high-intensity focused ultrasound; PD-L1, programmed cell death ligand 1.

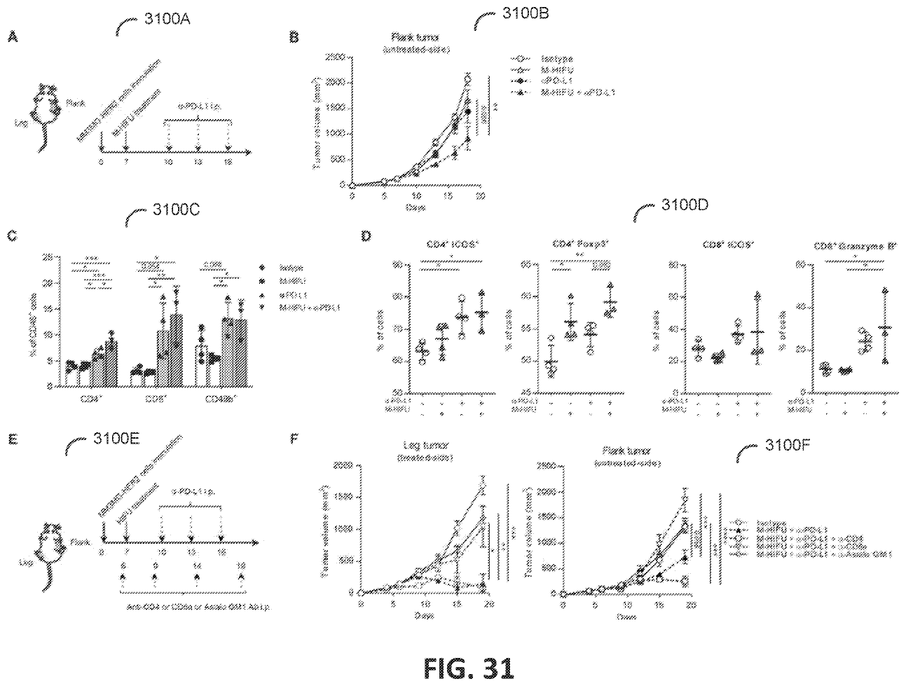

Based on the enhancement of systemic tumor antigen-specific immune responses by the addition of anti-PD-L1 (plot 2900C of FIG. 29A), it was hypothesized that PD-1/PD-L1 blockade would also improve the abscopal effect of M-HIFU. When MM3MG-HER2 tumors were treated with M-HIFU and administered intraperitoneal anti-PD-L1 antibody (plot 3100A of FIG. 31), a greater growth suppression against distant untreated tumors than with either treatment alone in a more stringent bilateral tumor model was observed, where mice were implanted with a larger number of tumor cells (five times larger compared with plot 2900C of FIG. 29A) to the flank (plot 3100B of FIG. 31). To elucidate the change of TME in distant HIFU-untreated tumors induced by the combination treatment, distant tumors were analyzed by flow cytometry. As shown in plot 3100C of FIG. 31, M-HIFU monotherapy could not increase CD4 T/CD8 T/NK-cell infiltration in rapidly growing distant tumors, which might be the main reason for weaker antitumor effect on distant tumors. However, combination treatment as well as anti-PD-L1 monotherapy increased the proportion of CD4+, CD8+, and NK-cell populations in distant tumors, compared with the untreated control and M-HIFU monotherapy (plot 3100C of FIG. 31). Likewise, CD4 T cells again expressed higher levels of activation markers in these groups and granzyme B expression in CD8 T cells was significantly higher in distant tumors in the combination treatment group (plot 3100D of FIG. 31), suggesting the most enhanced cytotoxic function of CD8 T cells in combination group which lead to the strongest abscopal effect (plot 3100B of FIG. 31).

Finally, to confirm which immune cells were responsible for the efficacy against the distant tumor in the bilateral tumor model, the mice were treated with M-HIFU+anti-PD-L1 therapy in the presence of depleting antibody for CD4+, CD8+, or NK cells (plot 3100E of FIG. 31). In both leg and flank tumors, depletion of CD8+ or NK cells significantly abrogated the antitumor efficacy (plot 3100F of FIG. 31). Greater efficacy of the combination therapy against distant tumors in CD8+ cell-dependent fashion was confirmed also in the JC-HER3 tumor model. To clarify how depletion of immune cells affect TME of the distant tumors in mice treated with the combination therapy, the profile of tumor-infiltrating immune cells in distant tumors by flow cytometry was analyzed. Interestingly, systemic depletion of CD8+ or NK cells resulted in an intratumoral immune cell profile following M-HIFU+anti-PD-L1 that was similar to the profile of untreated control tumors and reduced ICOS expression and granzyme B production by CD8 T cells, suggesting that these two cell types not only are the key effectors but may also influence the immune composition of the TME. On the other hand, depletion of CD4+ cells induced significant increase and activation of CD8 T cells, and the decrease of macrophages and Treg in distant tumors, that may explain the stronger abscopal effect observed in the CD4+ depleted group. These results indicate that both CD8+ cells and NK cells play significant roles in the immune-based attack of distant tumors as well as HIFU-treated tumors in this M-HIFU+anti-PD-L1 combination therapy.

With respect to FIG. 31, the combination of M-HIFU and PD-1/PD-L1 blockades synergistically inhibited the distant tumor growth, depending on CD8+ as well as NK cells. Regarding plot 3100A, MM3MG-HER2 cells were injected into both the left leg (1×106 cells) and the right flank (5×105 cells) of BALB/c mice on day 0. A larger number of cells in the flank was implanted, compared with plot 2500C of FIG. 25, to create a more stringent model. Established leg tumors were treated with M-HIFU on day 7. Anti-PD-L1 antibody (100 μg/100 μL) or isotype control IgG (100 μg/100 μL) was injected intraperitoneally on days 10, 13 and 16. Regarding plot 3100B, the tumor growth curves of the distant flank tumor are shown. n=10 mice per each group, 40 mice in total. Regarding plots 3100C and 3100D, on day 18, distant flank tumors (HIFU untreated side) were collected and infiltrating immune cells were analyzed by flow cytometry. n=3 (combination group) or 4 (other groups). Regarding plot 3100C, the percentages of CD4+, CD8+ and CD49b+ cells in CD45+ cells are shown. Regarding plot 3100D, the expression of ICOS and Foxp3 on CD4+ cells and the expression of ICOS and granzyme B by CD8+ cells. Regarding plot 3100E, mice were treated with the combination of M-HIFU and anti-PD-L1 antibody in the same treatment schedule as plot 2800A of FIG. 28 with or without administration of depleting antibody for CD4+, CD8a+, and NK cells on days 6, 9, 14, and every 5 days until the end of the experiment. Regarding plot 3100F, the tumor growth curves of the leg tumor (left) and the flank tumor (right) are shown. n=5 mice per each group, 25 mice in total. Error bars represent SE in plots 3100B and 3100F. Error bars represent SD in plots 3100C and 3100D. *P<0.05, **P<0.01, ***P<0.001, ****P<0.0001. HER2, human ErbB-2; M-HIFU, mechanical high-intensity focused ultrasound; NK, natural killer; PD-1, programmed death-1; PD-L1, programmed cell death ligand 1.

Many approaches seeking to modify the TME to induce systemic antitumor immunity are under development, including intratumoral delivery of cytokines and oncolytic viruses, and physical tumor destruction using, among others, focused ultrasound. The traditional focused ultrasound approach creates coagulation necrosis by heating tissue (T-HIFU); however, it may not optimally induce systemic antitumor immunity due to denaturation of tumor proteins. Conventional T-HIFU modifies the TME, yet does not induce a memory systemic immune response. Therefore, M-HIFU, which causes cell death and extracellular matrix disruption by acoustic microcavitation rather than heating the tissue, was studied, and was previously observed to improve antigen presentation, resulting in more favorable immune responses.

In the present study, it was demonstrated that M-HIFU induces local and systemic immunity against BC, that in models is more potent than T-HIFU. This was evidenced by greater antigen presentation, induction of antigen specific T cells within splenocytes, and increased T cell infiltration at the site of M-HIFU-ablated as well as contralateral tumors. A potential criticism is that there are a wide variety of T-HIFU protocols which might induce quite different antitumor effects or antitumor immune responses compared with the T-HIFU technique disclosed herein Some present systems reported data using a form of T-HIFU (thermal ablative focused ultrasound) to treat poorly immunogenic 4T1 tumors. Although thermal ablation alone had limited impact on the intratumoral accumulation of activated T cells (attributed to the immunosuppressive TME), combination with gemcitabine resulted in enhanced tumor control and improved survival of mice in a CD8 and CD4 T cell-dependent fashion. T-HIFU of BCs in patients was reported to enhance infiltration of immune cells.

Recent studies have demonstrated enhanced immunogenicity of BC treated by T-HIFU in combination with systemic ICB or local application of toll-like receptor 9 agonist CpG. It is also possible that the timing of immune assays might have affected the outcome of the comparative analysis between the different treatments. For example, T-HIFU-treated tumors could be within the adaptive resistance/wound healing phase at the time of tissue collection in our study. Even taking these into account, it is believed that the data of the immune assays, which corresponded well to in vivo antitumor efficacy, indicate the overall capacities of these 2 HIFU modes in the induction of antitumor immunity. More preclinical and clinical studies may be necessary to assess and optimize the antitumor efficacy of HIFU for BC treatment.

Although M-HIFU could significantly inhibit the growth of treated tumors and lengthen the survival of single tumor-bearing mice, the antitumor activity generated against the untreated remote tumors was modest. One explanation observed was the upregulation of PD-L1 by myeloid cell populations including macrophages in M-HIFU-treated tumors, which may have resulted in the induction of insufficient systemic antitumor immunity. PD-L1 has been shown to exert constitutive negative signals in macrophages and induce an immunosuppressive phenotype. Anti-PD-L1 treatment by itself remodeled the macrophage compartment in tumors toward a more proinflammatory phenotype, mainly through increased IFN-γ levels in TME, resulting in enhanced T-cell activity. In the study disclosed above, administration of anti-PD-L1 antibody with M-HIFU enhanced the antitumor efficacy against remote tumors. Importantly, the data suggest that the net effect of combination treatment is a more favorable pattern of T and NK cell attraction and activation not only at the site of the HIFU-treated tumor, but also at distant untreated tumors. As described earlier, others have combined HIFU with pathogen-associated molecular patterns, such as CpG, to generate an abscopal effect. These data, in conjunction with this demonstration that tumor control is CD8+ T cell dependent, support the contention that HIFU induces antigen-specific CD8+ T-cell responses that can be enhanced by nonspecific immune stimulators.

Despite the substantial antitumor effect of the combination therapy, some distant tumors grew after an initial period of control. It was hypothesized that there are two potential explanations for the lack of complete control. First, following tumor apoptosis in response to M-HIFU plus anti-PD-L1, tissue repair within the altered TME might involve macrophage repolarization to M2 as previously observed. In the models combining M-HIFU and anti-PD-L1, macrophage genes involved in cell matrix adhesion and extracellular matrix organization, important for wound healing, were upregulated in addition to genes typically associated with the M1 phenotype. However, this phenomenon of increased intratumoral phagocytic macrophages with upregulated gene signatures for wound healing, ECM remodeling, and anti-inflammation in anti-CD47 antibody-treated murine BCs experiencing ongoing tumor regression has been observed.

Therefore, the tendency to upregulate gene expression consistent with a reparative phenotype may not be detrimental once the initial antitumor immune response is activated by M1 macrophages. Second, intratumoral Treg derived from infiltrating CD4+ T cells might counteract effector T-cell function. The flow cytometric analysis of M-HIFU-treated tumor demonstrates that the combination strategy increased infiltration of CD4+ T cells including a population with enhanced Foxp3 expression, consistent with Treg cells. The depletion of CD4+ Tregs enhanced the antitumor efficacy of the combination treatment locally and in HIFU-untreated distant tumors. One additional observation is that human BC is not traditionally considered an inflamed immunogenic tumor and is less responsive to immunotherapies, such as ICB, compared with other immunogenic solid tumors as melanoma and lung cancer. In this study, to monitor induction of tumor-antigen specific immune response by HIFU treatment, tumor models that were engineered to express OVA, HER2 or HER3 antigen were used Forced expression of these foreign antigens would make the tumor cells more immunogenic, unlike human BC cases, and thus careful interpretation of the induced antigen-specific immune responses in these models was needed. Importantly, however, in one of the BC models, JC-HER3 tumors were implanted into HER3 transgenic mice that are immune tolerant for HER3 antigen, and the induction of anti-HER3 cellular immune responses with significant antitumor efficacy and abscopal effect by M-HIFU treatment was confirmed. These data suggest that M-HIFU was able to break immune tolerance to self-antigens.

In summary, the systems and methods described herein demonstrated a beneficial modification of the TME by M-HIFU and enhanced systemic antitumor efficacy by the combination of M-HIFU and anti-PD-L1 antibody, which resulted in significantly stronger growth suppression of distant tumors compared with M-HIFU or anti-PD-L1 monotherapy. To further enhance the antitumor efficacy and eradicate distant tumors by induced antitumor immunity, clinically applicable strategies may be tested to combine with M-HIFU plus anti-PD-L1, such as depletion of Tregs by targeting antibodies and enhancing the M1-biased TME by intratumoral IL-12 gene therapy.

### Example 7—Gene Delivery Using Sonoporation Energy|_{[HJA(7]}

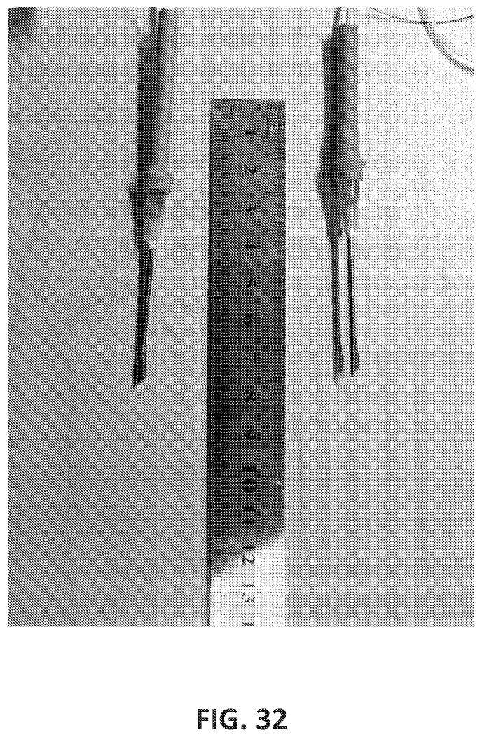

This example demonstrates gene/mRNA delivery by sonoporation into tumor and non-tumor tissue, including normal tissues. An in vivo test with sonoporation was conducted with murine CT26 cells, dual flank injection, BALB/c mice. An example sonoporation transducer is illustrated in FIG. 32. The sonication parameters were 1.1 MHz for frequency, peak negative pressure (PNP): 0.1 MPa, 0.4 MPa, 0.7 MPa, and 200 cycles.

In a first sequence, the transducer was inserted into a target, a solution was injected into the target for 60 s, and then sonication was applied for 30 s. In a second sequence, the transducer was inserted into the target, a solution was injected for 60 s, and then sonication was applied for 30 s, a solution was injected again for 30 s, and then sonication was applied for 30 s.

The injected solution comprised a microbubble concentration of 7.68×10⁸ bubbles/mL and a plasmid DNA concentration of 20 μg/mL. The solution was injected at a rate of 0.1 mL/min.

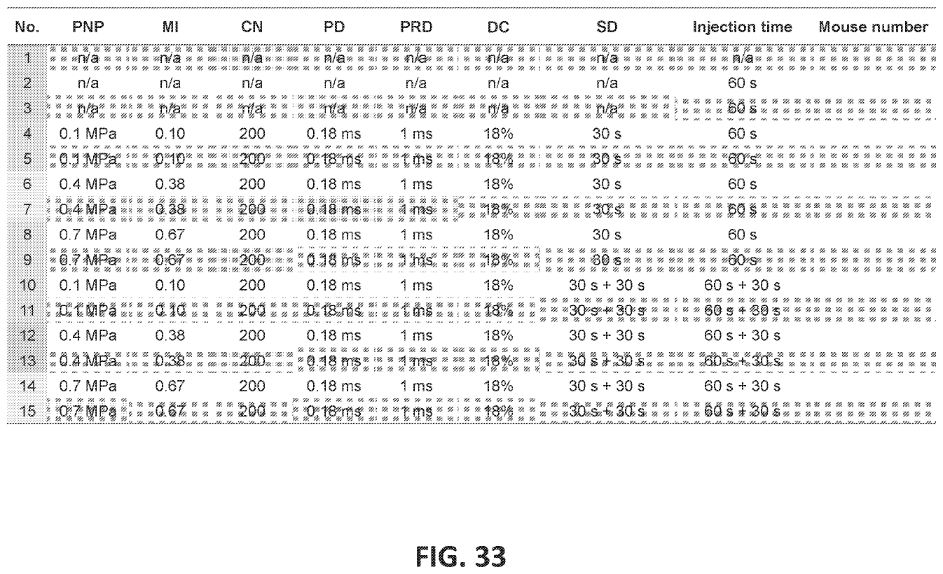

There were 12 test groups consisting of three (various PNP)×two (different sequence)×two (repetition). The control group (i.e., untreated, w/plasmid only, w/plasmid and w/MBs) was three. The test conditions are shown in FIG. 33. No. 1 in FIG. 33 is the negative-negative control group, i.e., untreated; No. 2 in FIG. 33 is the negative control group, i.e., w/plasmid only; and No. 3 in FIG. 33 is the negative control group, i.e., w/plasmid and w/MBs.

Positive controls included Ad-Luciferase intratumoral injection and GFP-luciferase plasmid injection and electroporation (e.g., same concentration of plasmid, same plasmid, same timepoint). To eliminate the risk of missing the part of the tumor that was expressing the plasmid, the entire tumor was lysed.

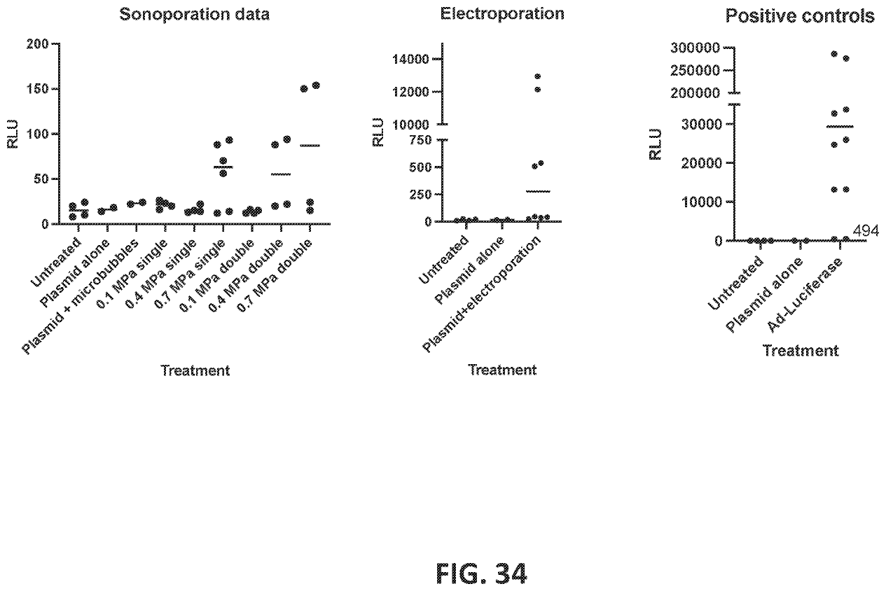

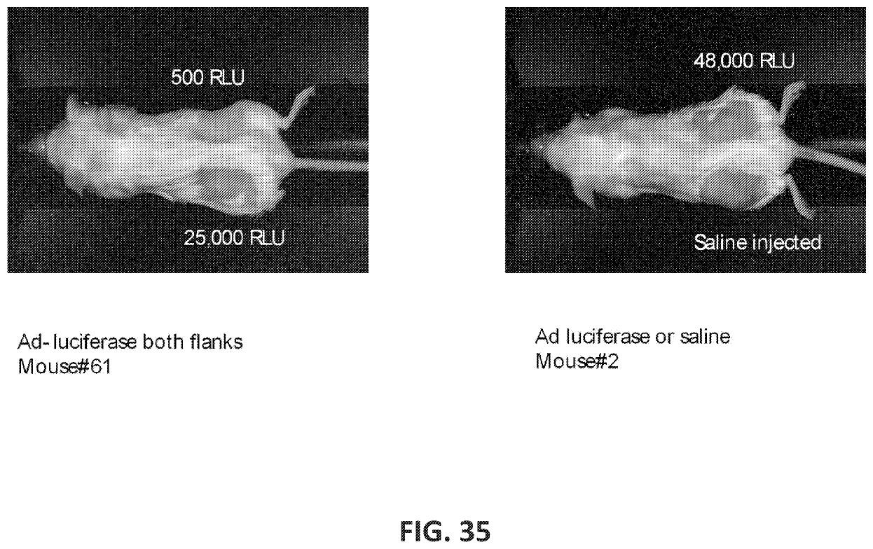

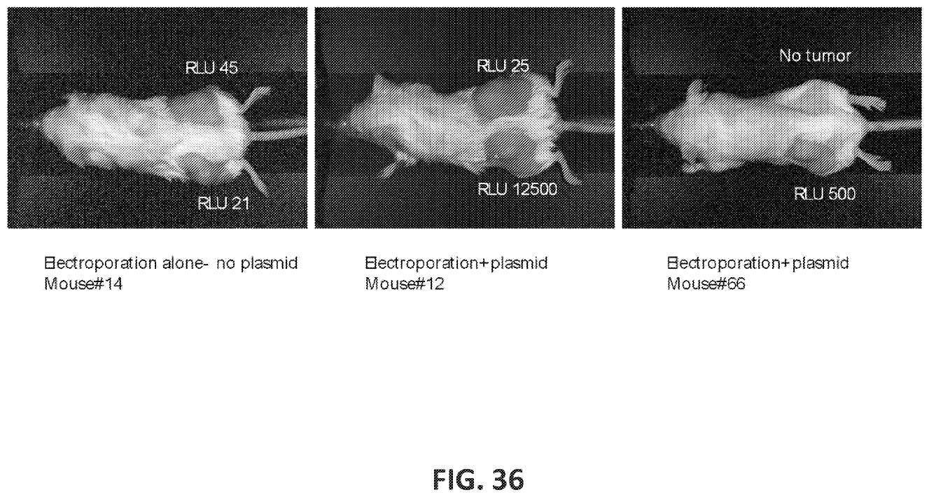

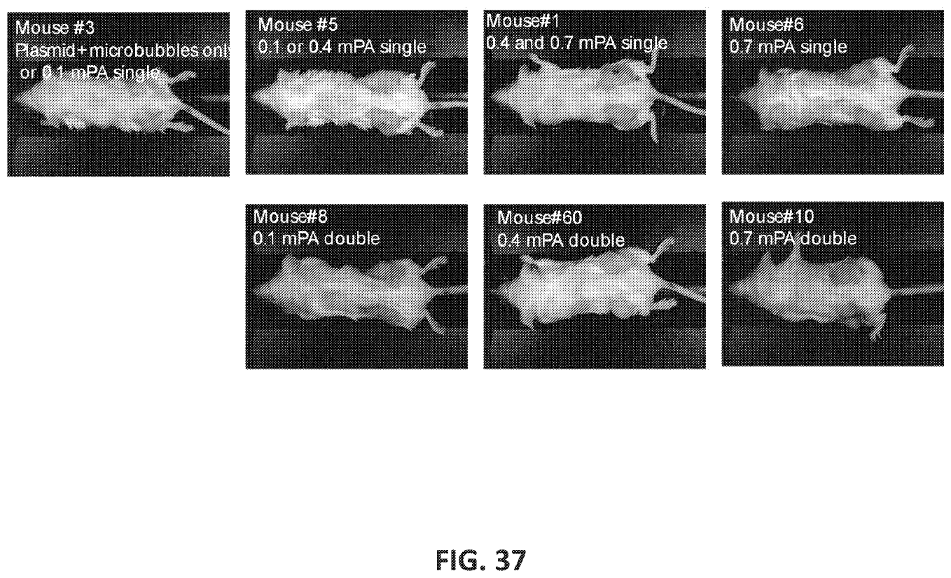

Data was measured with a luminometer and is shown in FIGS. 34-37. FIG. 35 illustrates data with Ad-Luciferase or saline as the intratumoral injection. FIG. 36 illustrates data with electroporation applied. FIG. 37 illustrates data with sonoporation applied.

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A catheter comprising an elongated hollow tube, a first lumen in the elongated hollow tube, a second lumen in the elongated hollow tube, a transducer positioned within the elongated hollow tube and adjacent to the first lumen or the second lumen, the ultrasonic transducer configured to emit ultrasound waves through the lumen to a target, and a needle positioned within the elongated hollow tube and configured to extend from the first lumen or the second lumen to enter the target to deliver a therapy to the target.

Clause 2. The catheter of clause 1, wherein the therapy is delivered to the target while the ultrasound waves are delivered to the target.

Clause 3. The catheter of clause 1 or 2, wherein the transducer is configured to deliver intracorporeal sonoporation to the target.

Clause 4. The catheter of any one of clauses 1-3, wherein the intracorporeal sonoporation generates acoustic cavitation at the target to induce formation of pores in a cell membrane of the target to increase permeability of the target.

Clause 5. The catheter of any one of clauses 1-4, wherein the target is malignant tissue, tumor tissue, peritumoral tissue, non-malignant tissue, hematologie cells, or immune cells.

Clause 6. The catheter of any one of clauses 1-5, wherein the needle is configured to deliver immunotherapy directly within the target.

Clause 7. The catheter of any one of clauses 1-5, wherein the needle is configured to deliver gene therapy directly within the target.

Clause 8. The catheter of any one of clauses 1-5, wherein the needle is configured to deliver one or more therapeutics directly within the target.

Clause 9. The catheter of clause 1, wherein the transducer is configured to deliver high-pressure acoustic bursts of focused ultrasound waves toward the target to generate acoustic cavitation at the target.

Clause 10. The catheter of clause 9, wherein the acoustic cavitation at the target provides expansion and collapse of microbubbles to release high-pressure cavitation energy to disrupt extra-cellular matrix of the target.

Clause 11. The catheter of clause 9 or 10, wherein the target is malignant tissue, tumor tissue, peritumoral tissue, non-malignant tissue, hematologic cells, or immune cells.

Clause 12. The catheter of any one of clauses 9-11, wherein the needle is configured to deliver immunotherapy directly within the target.

Clause 13. The catheter of any one of clauses 9-11, wherein the needle is configured to deliver gene therapy directly within the target.

Clause 14. The catheter of any one of clauses 9-11, wherein the needle is configured to deliver one or more therapeutics directly within the target.

Clause 15. The catheter of any one of clauses 1-14, wherein the transducer is configured to deliver energy to the target that results in acoustic pressure (peak negative) applied to the target within a range of 5 MPa to greater than 50 MPa.

Clause 16. The catheter of clause 15, wherein the acoustic pressure applied to the target is within a range of 15 MPa to 30 MPa.

Clause 17. The catheter of clause 15 or 16, wherein the acoustic pressure applied to the target is within a range of 20 MPa to 45 MPa.

Clause 18. The catheter of any one of clauses 1-17, wherein the transducer is configured to ablate the target.

Clause 19. The catheter of any one of clauses 1-18, wherein the target is adjacent to or within a gas-filled anatomical organ.

Clause 20. The catheter of clause 19, wherein the gas-filled anatomical organ is a lung, a bowel, an airway, or a bladder.

Clause 21. The catheter of any one of clauses 1-20, wherein the transducer includes a plurality of electrodes positioned adjacent to one another in a non-linear orientation.

Clause 22. The catheter of clause 21, wherein the plurality of electrodes is positioned to form a radius of curvature in a range of 5 mm to 10 mm.

Clause 23. The catheter of clause 21 or 22, wherein each of the plurality of electrodes have an aperture size of about 1.4 mm×1.8 mm.

Clause 24. The catheter of any one of clauses 21-23, wherein at least one of the plurality of electrodes comprises a piezoelectric plate with a thickness of less than about 50 μm to about 500 μm.

Clause 25. The catheter of any one of clauses 1-24, wherein the catheter is a 6-7 Fr catheter.

Clause 26. The catheter of any one of clauses 1-25, wherein the catheter is an 8-10 Fr catheter.

Clause 27. A method of treating a malignant tumor, the method comprising inserting a catheter of claim 1 within a subject and toward the malignant tumor, activating the transducer to deliver energy through the first lumen to the malignant tumor that results in acoustic peak negative pressure applied to the malignant tumor within a range of 10 MPa to 40 MPa, and activating the needle to extend from the second lumen to enter the malignant tumor to deliver a therapeutically effective amount of a pharmaceutical composition.

Clause 28. The method of clause 27, wherein the pharmaceutical composition includes at least one selected from a group consisting of cytokines, chemokines, and other biologic proteins (IL-12 and the like), oncolytic viruses, CAR-T cells, TILs, cDNA, mRNA, self-replicating RNA, proteins, immuno-suppressants, anti-inflammatories-proliferatives, anti-migratory agents, anti-fibrotic agents, pro-apoptotics, vasodilators, calcium channel blockers, anti-neoplastics, anti-cancer agents, antibodies, anti-thrombotic agents, anti-platelet agents, IIb/IIIa agents, antiviral agents, mTOR (mammalian target of rapamycin) inhibitors, and non-immunosuppressant agents.

Clause 29. The method of clause 27 or 28, wherein the pharmaceutical composition incudes an alkylating agent for targeting DNA.

Clause 30. A system for the treatment of a target, the system comprising an ultrasound energy source, a device coupled to the ultrasound energy source and configured and arranged to direct the ultrasound energy to a target, release one or more microbubbles, and release one or more therapeutic agents, in which the microbubbles burst upon receiving the ultrasound energy thereby disrupting the target and surrounding extracellular matrix (ECM) of the target and allowing for the one or more therapeutic agents to be delivered within the target.

Clause 31. The system of clause 30, wherein the one or more microbubbles are configured and arranged to contain the one or more therapeutic agents, wherein the one or more microbubbles burst upon receiving the ultrasound energy thereby releasing the one or more therapeutic agents within the target.

Clause 32. The system of clause 30 or 31, wherein the device further comprises a configuration and arrangement to release one or more contrast agents.

Clause 33. The system of any one of clauses 30-32, wherein the ultrasound energy comprises high intensity focused ultrasound (HIFU).

Clause 34. The system of any one of clauses 30-32, wherein the ultrasound energy comprises histotripsy.

Clause 35. The system of any one of clauses 30-34, wherein the target is malignant tissue, tumor tissue, peritumoral tissue, non-malignant tissue, hematologic cells, or immune cells.

Clause 36. A method for the delivery of a drug to a target, the method comprising inserting a catheter within a subject and toward the target, the catheter including the device of clause 30, generating an ultrasound energy output near the target, supplying one or more microbubbles near the target, and supplying one or more therapeutic agents through the catheter, wherein the microbubbles burst upon receiving the ultrasound energy thereby disrupting the target and surrounding extracellular matrix (ECM) and allowing for the one or more therapeutic agents to be delivered within the target thereby treating the target.

Clause 37. The method of clause 36, wherein the one or more microbubbles are configured and arranged to contain the one or more therapeutic agents, wherein when the one or more microbubbles burst upon receiving the ultrasound energy, thereby releasing the one or more therapeutic agents within the target.

Clause 38. The method of clause 36 or 37, wherein the method further comprises releasing one or more contrast agents.

Clause 39. The method of any one of clauses 36-38, wherein the one or more therapeutic agents are selected from the group consisting of cytokines, chemokines, and other biologic proteins (IL-12 and the like), oncolytic viruses, CAR-T cells, TILs, cDNA, mRNA, self-replicating RNA, proteins, immuno-suppressants, anti-inflammatoirenti-proliferatives, anti-migratory agents, anti-fibrotic agents, proapoptotics, vasodilators, calcium channel blockers, anti-neoplastics, anti-cancer agents, antibodies, anti-thrombotic agents, anti-platelet agents, IIb/IIIa agents, antiviral agents, mTOR (mammalian target of rapamycin) inhibitors, non-immunosuppressant agents, and combinations thereof.

Clause 40. The method of any one of clauses 36-39, wherein the one or more therapeutic agents comprise a cytokine, a chemokine, or other biologic protein.

Clause 41. The method of clause 40, wherein the cytokine comprises IL-12.

What is claimed is:

1. A catheter comprising:
an elongated hollow tube;
a first lumen in the elongated hollow tube;
a second lumen in the elongated hollow tube;
a transducer positioned within the elongated hollow tube and adjacent to the first lumen or the second lumen, the ultrasonic transducer configured to emit ultrasound waves through the lumen to a target; and
a needle positioned within the elongated hollow tube and configured to extend from the first lumen or the second lumen to enter the target to deliver a therapy to the target.

2. The catheter of claim 1, wherein the therapy is delivered to the target while the ultrasound waves are delivered to the target.

3. The catheter of claim 1 or 2, wherein the transducer is configured to deliver intracorporeal sonoporation to the target.

4. The catheter of any one of claims 1-3, wherein the intracorporeal sonoporation generates acoustic cavitation at the target to induce formation of pores in a cell membrane of the target to increase permeability of the target.

5. The catheter of any one of claims 1-4, wherein the target is malignant tissue, tumor tissue, peritumoral tissue, non-malignant tissue, hematologic cells, or immune cells.

6. The catheter of any one of claims 1-5, wherein the needle is configured to deliver immunotherapy directly within the target.

7. The catheter of any one of claims 1-5, wherein the needle is configured to deliver gene therapy directly within the target.

8. The catheter of any one of claims 1-5, wherein the needle is configured to deliver one or more therapeutics directly within the target.

9. The catheter of claim 1, wherein the transducer is configured to deliver high-pressure acoustic bursts of focused ultrasound waves toward the target to generate acoustic cavitation at the target.

10. The catheter of claim 9, wherein the acoustic cavitation at the target provides expansion and collapse of microbubbles to release high-pressure cavitation energy to disrupt extra-cellular matrix of the target.

11. The catheter of claim 9 or 10, wherein the target is malignant tissue, tumor tissue, peritumoral tissue, non-malignant tissue, hematologic cells, or immune cells.

12. The catheter of any one of claims 9-11, wherein the needle is configured to deliver immunotherapy directly within the target.

13. The catheter of any one of claims 9-11, wherein the needle is configured to deliver gene therapy directly within the target.

14. The catheter of any one of claims 9-11, wherein the needle is configured to deliver one or more therapeutics directly within the target.

15. The catheter of any one of claims 1-14, wherein the transducer is configured to deliver energy to the target that results in acoustic pressure (peak negative) applied to the target within a range of 5 MPa to greater than 50 MPa.

16. The catheter of claim 15, wherein the acoustic pressure applied to the target is within a range of 15 MPa to 30 MPa.

17. The catheter of claim 15 or 16, wherein the acoustic pressure applied to the target is within a range of 20 MPa to 45 MPa.

18. The catheter of any one of claims 1-17, wherein the transducer is configured to ablate the target.

19. The catheter of claim any one of claims 1-18, wherein the target is adjacent to or within a gas-filled anatomical organ.

20. The catheter of claim 19, wherein the gas-filled anatomical organ is a lung, a bowel, an airway, or a bladder.

21. The catheter of any one of claims 1-20, wherein the transducer includes a plurality of electrodes positioned adjacent to one another in a non-linear orientation.

22. The catheter of claim 21, wherein the plurality of electrodes is positioned to form a radius of curvature in a range of 5 mm to 10 mm.

23. The catheter of claim 21 or 22, wherein each of the plurality of electrodes have an aperture size of about 1.4 mm×1.8 mm.

24. The catheter of any one of claims 21-23, wherein at least one of the plurality of electrodes comprises a piezoelectric plate with a thickness of less than about 50 μm to about 500 μm.

25. The catheter of any one of claims 1-24, wherein the catheter is a 6-7 Fr catheter.

26. The catheter of any one of claims 1-25, wherein the catheter is an 8-10 Fr catheter.

27. A method of treating a malignant tumor, the method comprising:
inserting a catheter of claim 1 within a subject and toward the malignant tumor;
activating the transducer to deliver energy through the first lumen to the malignant tumor that results in acoustic peak negative pressure applied to the malignant tumor within a range of 10 MPa to 40 MPa; and
activating the needle to extend from the second lumen to enter the malignant tumor to deliver a therapeutically effective amount of a pharmaceutical composition.

28. The method of claim 27, wherein the pharmaceutical composition includes at least one selected from a group consisting of cytokines, chemokines, and other biologic proteins (IL-12 and the like), oncolytic viruses, CAR-T cells, TILs, cDNA, mRNA, self-replicating RNA, proteins, immuno-suppressants, anti-inflammatories, anti-proliferatives, anti-migratory agents, anti-fibrotic agents, pro-apoptotics, vasodilators, calcium channel blockers, anti-neoplastics, anti-cancer agents, antibodies, anti-thrombotic agents, anti-platelet agents, IIb/IIIa agents, antiviral agents, mTOR (mammalian target of rapamycin) inhibitors, and non-immunosuppressant agents.

29. The method of claim 27 or 28, wherein the pharmaceutical composition incudes an alkylating agent for targeting DNA.

30. A system for the treatment of a target, the system comprising:
an ultrasound energy source;
a device coupled to the ultrasound energy source and configured and arranged to:
direct the ultrasound energy to a target,
release one or more microbubbles, and
release one or more therapeutic agents, in which the microbubbles burst upon receiving the ultrasound energy thereby disrupting the target and surrounding extracellular matrix (ECM) of the target and allowing for the one or more therapeutic agents to be delivered within the target.

31. The system of claim 30, wherein the one or more microbubbles are configured and arranged to contain the one or more therapeutic agents, wherein the one or more microbubbles burst upon receiving the ultrasound energy thereby releasing the one or more therapeutic agents within the target.

32. The system of claim 30 or 31, wherein the device further comprises a configuration and arrangement to:
release one or more contrast agents.

33. The system of any one of claims 30-32, wherein the ultrasound energy comprises high intensity focused ultrasound (HIFU).

34. The system of any one of claims 30-32, wherein the ultrasound energy comprises histotripsy.

35. The system any one of claims 30-34, wherein the target is malignant tissue, tumor tissue, peritumoral tissue, non-malignant tissue, hematologic cells, or immune cells.

36. A method for the delivery of a drug to a target, the method comprising:
inserting a catheter within a subject and toward the target, the catheter including the device of claim 30;
generating an ultrasound energy output near the target;
supplying one or more microbubbles near the target; and
supplying one or more therapeutic agents through the catheter, wherein the microbubbles burst upon receiving the ultrasound energy thereby disrupting the target and surrounding extracellular matrix (ECM) and allowing for the one or more therapeutic agents to be delivered within the target thereby treating the target.

37. The method of claim 36 wherein the one or more microbubbles are configured and arranged to contain the one or more therapeutic agents, wherein when the one or more microbubbles burst upon receiving the ultrasound energy, thereby releasing the one or more therapeutic agents within the target.

38. The method of claim 36 or 37, wherein the method further comprises releasing one or more contrast agents.

39. The method of any one of claims 36-38 wherein the one or more therapeutic agents are selected from the group consisting of cytokines, chemokines, and other biologic proteins (IL-12 and the like), oncolytic viruses, CAR-T cells, TILs, cDNA, mRNA, self-replicating RNA, proteins, immuno-suppressants, anti-inflammatories, anti-proliferatives, anti-migratory agents, anti-fibrotic agents, proapoptotics, vasodilators, calcium channel blockers, anti-neoplastics, anti-cancer agents, antibodies, anti-thrombotic agents, anti-platelet agents, IIb/IIIa agents, antiviral agents, mTOR (mammalian target of rapamycin) inhibitors, non-immunosuppressant agents, and combinations thereof.

40. The method of any one of claims 36-39, wherein the one or more therapeutic agents comprise a cytokine, a chemokine, or other biologic protein.

41. The method of claim 40 wherein the cytokine comprises IL-12.
